(19) United States
(12) Patent Application Publication
(10) Pub. No.: US 2011/0207771 A1
(43) Pub. Date: Aug. 25, 2011

(54) N-(L-ALKYL-2-PHENYLETHYL)-CARBOXAMIDE DERIVATIVES AND USE THEREOF AS FUNGICIDES

(75) Inventors: Daniel Stierli, Stein (CH); John J. Taylor, Bracknell (GB); Harald Walter, Stein (CH); Paul Anthony Worthington, Bracknell (GB); Ramya Rajan, Goa (IN)

(73) Assignee: SYNGENTA CROP PROTECTION, INC., Greensboro, NC (US)

(21) Appl. No.: 12/303,763

(22) PCT Filed: Jun. 6, 2007
(86) PCT No.: PCT/EP2007/005020

§ 371 Date: Dec. 8, 2008

(30) Foreign Application Priority Data
Jun. 8, 2006 (EP) 06011812.2

(51) Int. Cl. A01N 43/56; C07D 231/14; C07D 401/12; C07D 277/56; A01N 43/78; C07D 417/12; C07D 207/34; A01N 43/36; A01N 43/40; C07D 249/04; A01N 43/647; A01N 43/42; A01P 3/00

(52) U.S. Cl. 514/314; 548/374.1; 514/406; 546/275.4; 514/341; 548/200; 514/365; 546/269.7; 514/342; 548/537; 514/423; 546/279.1; 514/343; 548/255; 514/359; 546/268.4; 514/340; 546/175

(57) ABSTRACT

Compounds of the formula (I) in which the substituents are as defined in claim 1 are suitable for use as microbiocides.

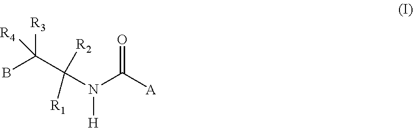

The present invention relates to novel microbiocidally active, in particular fungicidally active, ethyl amides. It further relates to intermediates used in the preparation of these compounds, to compositions which comprise these compounds and to their use in agriculture or horticulture for controlling or preventing infestation of plants by phytopathogenic microorganisms, preferably fungi.

N-[2-(pyridinyl)ethyl]-carboxamide derivatives and their use as fungicides are described in WO 04/074280, WO 05/085238, WO 06/008193 and WO 06/008194. Thiazole-5-carboxylic acid amide derivatives and their use as microbiocides or pest-controlling agents are described in EP-0-279-239 and JP-2001-342183. Pyrazole-4-carboxylic acid amide derivatives and their use as pest-controlling agents are described in JP-2001-342179. Similar compounds are also known in other fields of technology, for example, the use of thiazole-5-carboxylic acid amide derivatives as herbicide antagonists is described in EP-0-335-831 and the use of pyrazole-amides and sulfonamides as pain therapeutics is described in WO 03/037274.

It has been found that novel ethyl amides have microbiocidal activity.

The present invention thus provides compounds of the formula I

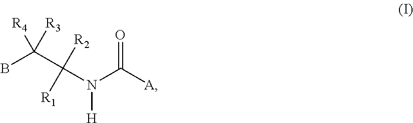

wherein
R₁, R₂, R₃ and R₄ independently of each other stand for hydrogen, halogen, nitro, C₁-C₆alkyl, which is unsubstituted or substituted by one or more substituents R₅, C₃-C₆cycloalkyl, which is unsubstituted or substituted by one or more substituents R₅, C₂-C₆alkenyl, which is unsubstituted or substituted by one or more substituents R₅, C₂-C₆alkynyl, which is unsubstituted or substituted by one or more substituents R₅;
or R₁ and R₂ together are a C₂-C₅alkylene group, which is unsubstituted or substituted by one or more C₁-C₆alkyl groups;
or R₃ and R₄ together are a C₂-C₅alkylene group, which is unsubstituted or substituted by one or more C₁-C₆alkyl groups;
each R₅ independently of each other stands for halogen, nitro, C₁-C₆alkoxy, C₁-C₆halogenalkoxy, C₃-C₆cycloalkyl, C₁-C₆alkylthio, C₁-C₆halogenalkylthio or —C(R^{a})═N(OR^{b});
R^{a} is hydrogen or C₁-C₆alkyl;
R^{b} is C₁-C₆alkyl;

### A is A₁

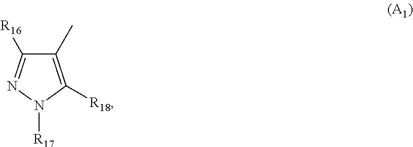

in which
R₁₆ is halogenmethyl;
R₁₇ is C₁-C₄alkyl, C₁-C₄halogenalkyl, C₁-C₄alkoxy-C₁-C₄alkyl or C₁-C₄halogenalkoxy-C₁-C₄alkyl; and
R₁₈ is hydrogen, halogen, cyano, nitro, C₁-C₄alkyl, C₁-C₄halogenalkyl, C₁-C₄halogenalkoxy, C₁-C₄alkoxy-C₁-C₄alkyl or C₁-C₄halogenalkoxy-C₁-C₄alkyl;

### or A is A₂

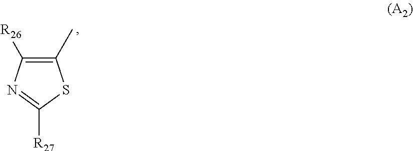

in which
R₂₆ is halogenmethyl; and
R₂₇ is C₁-C₄alkyl, C₁-C₄halogenalkyl, C₁-C₄alkoxy-C₁-C₄alkyl or C₁-C₄halogenalkoxy-C₁-C₄alkyl;

### or A is A₃

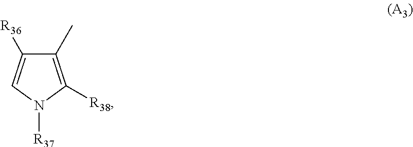

in which
R₃₆ is halogenmethyl;
R₃₇ is C₁-C₄alkyl, C₁-C₄halogenalkyl, C₁-C₄alkoxy-C₁-C₄alkyl or C₁-C₄halogenalkoxy-C₁-C₄alkyl; and
R₃₈ is hydrogen, halogen, cyano, nitro, C₁-C₄alkyl, C₁-C₄halogenalkyl, C₁-C₄halogenalkoxy, C₁-C₄alkoxy-C₁-C₄alkyl or C₁-C₄halogenalkoxy-C₁-C₄alkyl;

### or A is A₄

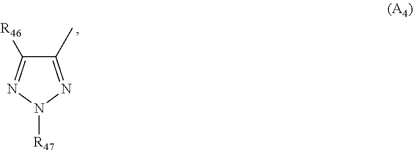

in which
R₄₆ is halogenmethyl; and
R₄₇ is C₁-C₄alkyl, C₁-C₄halogenalkyl, C₁-C₄alkoxy-C₁-C₄alkyl or C₁-C₄halogenalkoxy-C₁-C₄alkyl;
B is a phenyl, naphthyl or quinolinyl group, which is substituted by one or more substituents R₈;
each substituent R₈ independently of each other stands for halogen, C₁-C₆haloalkoxy, C₁-C₆haloalkylthio, cyano, nitro, —C(R^{c})═N(OR^{d}), C₁-C₆alkyl, which is unsubstituted or substituted by one or more substituents R₉, C₃-C₆cycloalkyl, which is unsubstituted or substituted by one or more substituents R₉, C₆-C₁₄bicycloalkyl, which is unsubstituted or substituted by one or more substituents R₉, C₂-C₆alkenyl, which is unsubstituted or substituted by one or more substituents R₉, C₂-C₆alkynyl, which is unsubstituted or substituted by one or more substituents R₉, phenyl, which is unsubstituted or substituted by one or more substituents R₉, phenoxy, which is unsubstituted or substituted by one or more substituents R₉ or pyridinyloxy, which is unsubstituted or substituted by one or more substituents R₉;
each R^{c} is independently of each other hydrogen or C₁-C₆alkyl;
each R^{d} is independently of each other C₁-C₆alkyl;
each R₉ is independently of each other halogen, nitro, C₁-C₆alkoxy, C₁-C₆halogenalkoxy, C₁-C₆alkylthio, C₁-C₆halogenalkylthio, C₃-C₆alkenyloxy, C₃-C₆alkynyloxy or —C(R^{e})═N(OR);
each R^{e} is independently of each other hydrogen or C₁-C₆alkyl;
each R^{f} is independently of each other C₁-C₆alkyl;
and tautomers/isomers/enantiomers of these compounds.

The alkyl groups occurring in the definitions of the substituents can be straight-chain or branched and are, for example, methyl, ethyl, n-propyl, n-butyl, n-pentyl, n-hexyl, iso-propyl, n-butyl, sec-butyl, iso-butyl or tert-butyl. Alkoxy, alkenyl and alkynyl radicals are derived from the alkyl radicals mentioned. The alkenyl and alkynyl groups can be mono- or di-unsaturated.

The cycloalkyl groups occurring in the definitions of the substituents are, for example, cyclopropyl, cyclobutyl, cyclopentyl or cyclohexyl.

The bicycloalkyl groups occurring in the definitions of the substituents are, depending on the ring size, bicyclo[2.1.1]hexane, bicyclo[2.2.1]heptane, bicyclo[2.2.2]octane, bicyclo[3.2.1]octane, bicyclo[3.2.2]nonane, bicyclo[4.2.2]decane, bicyclo[4.3.2]undecane, adamantane and the like.

Halogen is generally fluorine, chlorine, bromine or iodine, preferably fluorine, bromine or chlorine. This also applies, correspondingly, to halogen in combination with other meanings, such as halogenalkyl or halogenalkoxy.

Halogenalkyl groups preferably have a chain length of from 1 to 4 carbon atoms. Halogenalkyl is, for example, fluoromethyl, difluoromethyl, trifluoromethyl, chloromethyl, dichloromethyl, trichloromethyl, 2,2,2-trifluoroethyl, 2-fluoroethyl, 2-chloroethyl, pentafluoroethyl, 1,1-difluoro-2,2,2-trichloroethyl, 2,2,3,3-tetrafluoroethyl and 2,2,2-trichloroethyl; preferably trichloromethyl, difluorochloromethyl, difluoromethyl, trifluoromethyl and dichlorofluoromethyl.

Suitable halogenalkenyl groups are alkenyl groups which are mono- or polysubstituted by halogen, halogen being fluorine, chlorine, bromine and iodine and in particular fluorine and chlorine, for example 2,2-difluoro-1-methylvinyl, 3-fluoropropenyl, 3-chloropropenyl, 3-bromopropenyl, 2,3,3-trifluoropropenyl, 2,3,3-trichloropropenyl and 4,4,4-trifluorobut-2-en-1-yl.

Suitable halogenalkynyl groups are, for example, alkynyl groups which are mono- or polysubstituted by halogen, halogen being bromine, iodine and in particular fluorine and chlorine, for example 3-fluoropropynyl, 3-chloropropynyl, 3-bromopropynyl, 3,3,3-trifluoro-propynyl and 4,4,4-trifluorobut-2-yn-1-yl.

Alkoxy is, for example, methoxy, ethoxy, propoxy, i-propoxy, n-butoxy, isobutoxy, sec-butoxy and tert-butoxy; preferably methoxy and ethoxy. Halogenalkoxy is, for example, fluoromethoxy, difluoromethoxy, trifluoromethoxy, 2,2,2-trifluoroethoxy, 1,1,2,2-tetrafluoroethoxy, 2-fluoroethoxy, 2-chloroethoxy, 2,2-difluoroethoxy and 2,2,2-trichloroethoxy; preferably difluoromethoxy, 2-chloroethoxy and trifluoromethoxy. Alkylthio is, for example, methylthio, ethylthio, propylthio, isopropylthio, n-butylthio, isobutylthio, sec-butylthio or tert-butylthio, preferably methylthio and ethylthio.

Alkoxyalkyl is, for example, methoxymethyl, methoxyethyl, ethoxymethyl, ethoxyethyl, n-propoxymethyl, n-propoxyethyl, isopropoxymethyl or isopropoxyethyl.

In the context of the present invention “substituted by one or more substituents” in the definition of substituents R₁, R₂, R₃, R₄ and R₈, means typically, depending on the chemical structure of substituents R₁, R₂, R₃, R₄ and R₈, monosubstituted to nine-times substituted, preferably monosubstituted to five-times substituted, more preferably mono-, double- or triple-substituted.

In the context of the present invention “substituted by one or more substituents” in the definition of substituent B, means typically, depending on the chemical structure of substituent B, monosubstituted to seven-times substituted, preferably monosubstituted to five-times substituted, more preferably mono-, double- or triple-substituted.

The compounds of the formula I may occur in different tautomeric forms. For example, compounds of formula I exist in the tautomeric forms I_{I} and I_{II}:

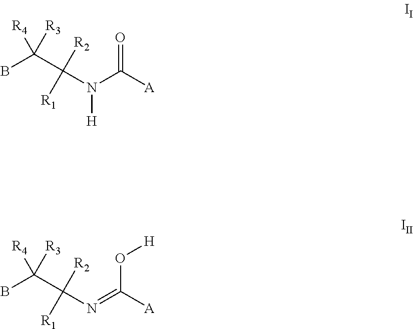

The invention covers all those tautomeric forms and mixtures thereof.

The present invention preferably provides compounds of the formula I

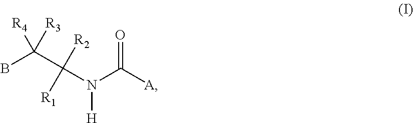

wherein

R₁, R₂, R₃ and R₄ independently of each other stand for hydrogen, halogen, nitro, C₁-C₆alkyl, which is unsubstituted or substituted by one or more substituents R₅, C₃-C₆cycloalkyl, which is unsubstituted or substituted by one or more substituents R₅, C₂-C₆alkenyl, which is unsubstituted or substituted by one or more substituents R₅ or C₂-C₆alkynyl, which is unsubstituted or substituted by one or more substituents R₅;

or R₁ and R₂ together are a C₂-C₅alkylene group, which is unsubstituted or substituted by one or more C₁-C₆alkyl groups;
or R₃ and R₄ together are a C₂-C₅alkylene group, which is unsubstituted or substituted by one or more C₁-C₆alkyl groups;
each R₅ independently of each other stands for halogen, nitro, C₁-C₆alkoxy, C₁-C₆halogenalkoxy, C₃-C₆cycloalkyl, C₁-C₆alkylthio, C₁-C₆halogenalkylthio or —C(R^{a})═N(OR^{b});
R^{a} is hydrogen or C₁-C₆alkyl;
R^{b} is C₁-C₆alkyl;

### A is A₁

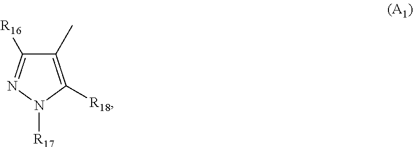

in which
R₁₆ is halogenmethyl;
R₁₇ is C₁-C₄alkyl, C₁-C₄halogenalkyl, C₁-C₄alkoxy-C₁-C₄alkyl or C₁-C₄halogenalkoxy-C₁-C₄alkyl; and
R₁₈ is hydrogen, halogen, cyano, nitro, C₁-C₄alkyl, C₁-C₄halogenalkyl, C₁-C₄halogenalkoxy, C₁-C₄alkoxy-C₁-C₄alkyl or C₁-C₄halogenalkoxy-C₁-C₄alkyl;

### or A is A₂

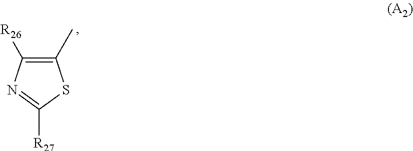

in which
R₂₆ is halogenmethyl; and
R₂₇ is C₁-C₄alkyl, C₁-C₄halogenalkyl, C₁-C₄alkoxy-C₁-C₄alkyl or C₁-C₄halogenalkoxy-C₁-C₄alkyl;

### or A is A₃

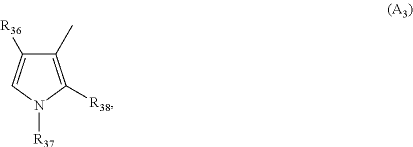

in which
R₃₆ is halogenmethyl;
R₃₇ is C₁-C₄alkyl, C₁-C₄halogenalkyl, C₁-C₄alkoxy-C₁-C₄alkyl or C₁-C₄halogenalkoxy-C₁-C₄alkyl; and
R₃₈ is hydrogen, halogen, cyano, nitro, C₁-C₄alkyl, C₁-C₄halogenalkyl, C₁-C₄halogenalkoxy, C₁-C₄alkoxy-C₁-C₄alkyl or C₁-C₄halogenalkoxy-C₁-C₄alkyl;

### or A is A₄

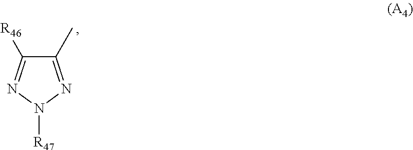

in which
R₄₆ is halogenmethyl; and
R₄₇ is C₁-C₄alkyl, C₁-C₄halogenalkyl, C₁-C₄alkoxy-C₁-C₄alkyl or C₁-C₄halogenalkoxy-C₁-C₄alkyl;
B is a phenyl, naphthyl or quinolinyl group, which is substituted by one or more substituents R₈;
each substituent R₈ independently of each other stands for halogen, C₁-C₆haloalkoxy, C₁-C₆haloalkylthio, cyano, nitro, —C(R^{c})═N(OR^{d}), C₁-C₆alkyl, which is unsubstituted or substituted by one or more substituents R₉, C₃-C₆cycloalkyl, which is unsubstituted or substituted by one or more substituents R₉, C₆-C₁₄bicycloalkyl, which is unsubstituted or substituted by one or more substituents R₉, C₂-C₆alkenyl, which is unsubstituted or substituted by one or more substituents R₉, C₂-C₆alkynyl, which is unsubstituted or substituted by one or more substituents R₉, phenyl, which is unsubstituted or substituted by one or more substituents R₉;
each R^{c} is independently of each other hydrogen or C₁-C₆alkyl;
each R^{d} is independently of each other C₁-C₆alkyl;
each R₉ is independently of each other halogen, nitro, C₁-C₆alkoxy, C₁-C₆halogenalkoxy, C₁-C₆alkylthio, C₁-C₆halogenalkylthio, C₃-C₆alkenyloxy, C₃-C₆alkynyloxy or —C(R^{e})═N(OR^{f});
each R^{e} is independently of each other hydrogen or C₁-C₆alkyl;
each R^{f} is independently of each other C₁-C₆alkyl;
and tautomers/isomers/enantiomers of these compounds.

In a preferred group of compounds R₁, R₂, R₃ and R₄ independently of each other stands for hydrogen, halogen, nitro, C₁-C₆alkyl, which is unsubstituted or substituted by one or more substituents R₅, C₃-C₆cycloalkyl, which is unsubstituted or substituted by one or more substituents R₅, C₂-C₆alkenyl, which is unsubstituted or substituted by one or more substituents R₅ or C₂-C₆alkynyl, which is unsubstituted or substituted by one or more substituents R₅; or R₁ and R₂ together are a C₂alkylene, which is unsubstituted or substituted by one or more C₁-C₆alkyl groups; or R₃ and R₄ together are a C₂alkylene group, which is unsubstituted or substituted by one or more C₁-C₆alkyl groups.

In a preferred group of compounds R₁, R₂, R₃ and R₄ independently of each other stands for hydrogen, halogen, nitro, C₁-C₆alkyl, which is unsubstituted or substituted by one or more substituents selected from halogen, cyano, C₁-C₆alkoxy and C₁-C₆halogenalkoxy; more preferably R₁, R₂, R₃ and R₄ independently of each other stands for hydrogen, halogen or C₁-C₆alkyl, which is unsubstituted or substituted by one or more substituents selected from halogen and C₁-C₆alkoxy; most preferably R₁, R₂, R₃ and R₄ independently of each other stands for hydrogen, halogen, or C₁-C₆alkyl.

In a preferred group of compounds R₁ is hydrogen, halogen, C₁-C₆alkyl, C₁-C₆halogenalkyl or C₁-C₆alkoxy-C₁-C₆alkyl; R₂ is hydrogen, halogen, C₁-C₆alkyl, C₁-C₆halogenalkyl or C₁-C₆alkoxy-C₁-C₆alkyl; R₃ is hydrogen, halogen, C₁-C₆alkyl, C₁-C₆halogenalkyl or C₁-C₆alkoxy-C₁-C₆alkyl; and R₄ is hydrogen, halogen, C₁-C₆alkyl, C₁-C₆halogenalkyl or C₁-C₆alkoxy-C₁-C₆alkyl. Within said embodiment, preferably, R₁ is hydrogen, halogen or C₁-C₆alkyl; and R₂, R₃ and R₄ are each independently selected from hydrogen and C₁-C₆alkyl. Within said embodiment, more preferably R₂ and R₄ are hydrogen. In one embodiment R₂, R₃ and R₄ are hydrogen. In another embodiment, R₁, R₂, R₃ and R₄ are hydrogen.

In another preferred group of compounds R₃ is halogen; preferably fluoro.

In another preferred group of compounds R₁ and R₂ together are a C₂-C₅alkylene group.

In one group of preferred compounds R₁ is C₁-C₆alkyl or C₁-C₆haloalkyl. In further preferred compounds R₁ is C₁-C₆alkyl. In further preferred compounds R₁ is C₁-C₃alkyl, CF₃ or CF₂H, even further preferred methyl.

In further preferred compounds R₁ is CF₃.

In further preferred compounds R₁ is CF₂H.

In further preferred compounds R₁ is CFH₂.

In a preferred group of compounds R₁ stands for

halogen, nitro, C₁-C₆alkyl, which is unsubstituted or substituted by one or more substituents R₅, C₃-C₆cycloalkyl, which is unsubstituted or substituted by one or more substituents R₅, C₂-C₆alkenyl, which is unsubstituted or substituted by one or more substituents R₅ or C₂-C₆alkynyl, which is unsubstituted or substituted by one or more substituents R₅; and
R₂, R₃ and R₄ independently of each other stands for
hydrogen, halogen, nitro, C₁-C₆alkyl, which is unsubstituted or substituted by one or more substituents R₅, C₃-C₆cycloalkyl, which is unsubstituted or substituted by one or more substituents R₅, C₂-C₆alkenyl, which is unsubstituted or substituted by one or more substituents R₅ or C₂-C₆alkynyl, which is unsubstituted or substituted by one or more substituents R₅; or R₃ and R₄ together are a C₂alkylene group, which is unsubstituted or substituted by one or more C₁-C₆alkyl groups.

In a further preferred group of these compounds R₁ stands for halogen, nitro, C₁-C₆alkyl, which is unsubstituted or substituted by one or more substituents selected from halogen, cyano, C₁-C₆alkoxy and C₁-C₆halogenalkoxy; more preferably R₁ stands for halogen or C₁-C₆alkyl, which is unsubstituted or substituted by one or more substituents selected from halogen and C₁-C₆alkoxy; most preferably R₁ stands for halogen, or C₁-C₆alkyl; and

R₂, R₃ and R₄ independently of each other stands for
hydrogen, halogen, nitro, C₁-C₆alkyl, which is unsubstituted or substituted by one or more substituents selected from halogen, cyano, C₁-C₆alkoxy and C₁-C₆halogenalkoxy; more preferably R₂, R₃ and R₄ independently of each other stands for hydrogen, halogen or C₁-C₆alkyl, which is unsubstituted or substituted by one or more substituents selected from halogen and C₁-C₆alkoxy; most preferably R₂, R₃ and R₄ independently of each other stands for hydrogen, halogen, or C₁-C₆alkyl.

In a yet further preferred group of these compounds R₁ is halogen, C₁-C₆alkyl, C₁-C₆halogenalkyl or C₁-C₆alkoxy-C₁-C₆alkyl; R₂ is hydrogen, halogen, C₁-C₆alkyl, C₁-C₆halogenalkyl or C₁-C₆alkoxy-C₁-C₆alkyl; R₃ is hydrogen, halogen, C₁-C₆alkyl, C₁-C₆halogenalkyl or C₁-C₆alkoxy-C₁-C₆alkyl; and R₄ is hydrogen, halogen, C₁-C₆alkyl, C₁-C₆halogenalkyl or C₁-C₆alkoxy-C₁-C₆alkyl.

Within said embodiment, preferably, R₁ is halogen or C₁-C₆alkyl (even further preferred C₁-C₆alkyl); and R₂, R₃ and R₄ are each independently selected from hydrogen and C₁-C₆alkyl. Within said embodiment, more preferably R₂ and R₄ are hydrogen. In one embodiment R₂, R₃ and R₄ are hydrogen. In another preferred embodiment R₁ is C₁-C₆alkyl, preferably methyl, and R₂, R₃ and R₄ are hydrogen.

In another preferred group of compounds R₃ is halogen.

In a preferred group of compounds A is A₁.

In another preferred group of compounds A is A₂.

In another preferred group of compounds A is A₃.

In another preferred group of compounds A is A₄.

In a particular preferred group of compounds A is A₁, wherein R₁₈ is hydrogen. In another particular preferred group of compounds A is A₁, wherein R₁₆ is halomethyl, preferably R₁₆ is selected from CF₃, CF₂H and CFH₂; R₁₇ is C₁-C₄alkyl; and R₁₈ is hydrogen or halogen, preferably hydrogen.

Further preferred are compounds, wherein A is A₁ and R₁ is C₁-C₆alkyl.

In another particular preferred group of compounds A is A₂, wherein R₂₆ is halomethyl. preferably R₂₆ is selected from CF₃, CF₂H and CFH₂; and R₂₇ is C₁-C₄alkyl.

In yet another particular preferred group of compounds A is A₃, wherein R₃₆ is halomethyl, preferably R₃₆ is selected from CF₃, CF₂H and CFH₂; R₃₇ is C₁-C₄alkyl; and R₃₈ is hydrogen or halogen.

In yet another particular preferred group of compounds A is A₄, wherein R₄₈ halomethyl, preferably R₄₈ is selected from CF₃, CF₂H and CFH₂; and R₄₇ is C₁-C₄alkyl.

One embodiment of the invention is represented by compounds, wherein B is a phenyl group, which is substituted by one or more substituents R₈.

Within said embodiment, preferably B is a phenyl group, which is substituted by one, two or three substituents R₈; more preferably B is a phenyl group, which is substituted by one or two substituents R₈.

Also preferably, B is a phenyl group, that is substituted by at least one substituent R₈ in the para-position.

In a preferred group of compounds each substituent R₈ independently of each other stands for halogen, C₁-C₆haloalkoxy, C₁-C₆haloalkylthio, nitro, —C(R^{c})═N(OR^{d}), C₁-C₆alkyl, which is unsubstituted or substituted by one or more substituents R₉, C₂-C₆alkenyl, which is unsubstituted or substituted by one or more substituents R₉ or C₂-C₆alkynyl, which is unsubstituted or substituted by one or more substituents R₉.

In a preferred group of compounds each substituent R₈ independently of each other stands for halogen, nitro, —C(R^{c})═N(OR^{d}), C₁-C₆alkyl, which is unsubstituted or substituted by one or more substituents R₉, C₂-C₆alkenyl, which is unsubstituted or substituted by one or more substituents R₉ or C₂-C₆alkynyl, which is unsubstituted or substituted by one or more substituents R₉.

In a preferred group of compounds each substituent R₈ independently of each other stands for halogen, nitro, —C(R^{c})═N(OR^{d}), C₁-C₆alkyl, which is unsubstituted or substituted by one or more substituents R₉, C₂-C₆alkenyl, which is unsubstituted or substituted by one or more substituents R₉ or C₂-C₆alkynyl, which is unsubstituted or substituted by one or more substituents R₉.

In a preferred group of compounds each substituent R₈ independently of each other stands for halogen, C₁-C₆alkyl, which is unsubstituted or substituted by one or more substituents selected from halogen and C₁-C₆alkoxy or C₂-C₆alkynyl, which is unsubstituted or substituted by one or more substituents selected from halogen and C₁-C₆alkoxy.

In a preferred group of compounds, B is B₁

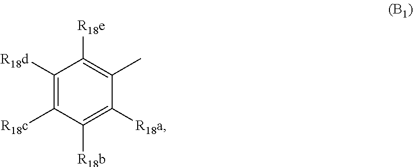

in which
R₁₈ₐ is hydrogen, halogen, cyano, C₁-C₆alkyl, C₂-C₆alkynyl, C₁-C₆alkoxy, C₁-C₆halogenalkyl, C₁-C₆halogenalkoxy or phenyl, which is unsubstituted or substituted by one or more halogens; R_{18b} is hydrogen, halogen, cyano, C₁-C₆alkyl, C₂-C₆alkynyl, C₁-C₆alkoxy, C₁-C₆halogenalkyl, C₁-C₆halogenalkoxy or phenyl, which is unsubstituted or substituted by one or more halogens; R_{18c} is hydrogen, halogen, cyano, C₁-C₆alkyl, C₂-C₆alkynyl, C₁-C₆alkoxy, C₁-C₆halogenalkyl, C₁-C₆halogenalkoxy or phenyl, which is unsubstituted or substituted by one or more halogens; R_{18d} is hydrogen, halogen, cyano, C₁-C₆alkyl, C₂-C₆alkynyl, C₁-C₆alkoxy, C₁-C₆halogenalkyl, C₁-C₆halogenalkoxy or phenyl, which is unsubstituted or substituted by one or more halogens; R₁₈ₑ is hydrogen, halogen, cyano, C₁-C₆alkyl, C₂-C₆alkynyl, C₁-C₆alkoxy, C₁-C₆halogenalkyl, C₁-C₆halogenalkoxy or phenyl, which is unsubstituted or substituted by one or more halogens; provided that at least one of R₁₈ₐ, R_{18b}, R_{18c}, R_{18d} and R₁₈ₑ is not hydrogen.

In one embodiment of the invention, R_{18b} and R_{18d} is hydrogen; and R₁₈ₐ, R_{18c} and R₁₈ₑ independently of one another are selected from hydrogen, halogen, cyano, C₂-C₆alkynyl, C₁-C₆halogenalkyl, C₁-C₆halogenalkoxy or phenyl, which is substituted halogen; provided that at least one of R₁₈ₐ, R_{18c} and R₁₈ₑ is not hydrogen.

In one embodiment of the invention, R_{18b} and R_{18d} is hydrogen; and R₁₈ₐ, R₁₈, and R₁₈ₑ independently of one another are selected from hydrogen, halogen, C₂-C₆alkynyl or C₁-C₆halogenalkyl; provided that at least one of R₁₈ₐ, R_{18c} and R₁₈ₑ is not hydrogen.

In one embodiment of the invention, R_{18b} and R_{18d} is hydrogen; R₁₈ₐ and R_{18c} independently of one another are selected from halogen, C₂-C₆alkynyl or C₁-C₆halogenalkyl, preferably from halogen, more preferably chloro; and R₁₈ₑ is selected from hydrogen, halogen, C₂-C₆alkynyl or C₁-C₆halogenalkyl, preferably from hydrogen or halogen, more preferably hydrogen or chloro.

Further preferred are compounds, wherein A is A₁ and R₁ is C₁-C₆alkyl and B is B₁.

Another embodiment of the invention is represented by compounds, wherein B is a naphthyl or quinolinyl group, which is substituted by one or more substituents R₈.

Another embodiment of the invention is represented by compounds, wherein B is a naphthyl group, which is substituted by one or more substituents R₈.

Within said embodiment, preferably B is a naphthyl group, which is substituted by one or two substituents R₈. Within said embodiment, in a preferred group of compounds each substituent R₈ independently of each other stands for halogen, C₁-C₆haloalkoxy, C₁-C₆alkyl, which is unsubstituted or substituted by one or more substituents selected from halogen and C₁-C₆alkoxy; C₂-C₆alkynyl, which is unsubstituted or substituted by one or more substituents selected from halogen and C₁-C₆alkoxy; or phenyl, which is unsubstituted or substituted by one or more halogens.

Another embodiment of the invention is represented by compounds, wherein B is a quinolinyl group, which is substituted by one or two substituents R₈. Within said embodiment, in a preferred group of compounds each substituent R₈ independently of each other stands for halogen, C₁-C₆haloalkoxy, C₁-C₆alkyl, which is unsubstituted or substituted by one or more substituents selected from halogen and C₁-C₆alkoxy; C₂-C₆alkynyl, which is unsubstituted or substituted by one or more substituents selected from halogen and C₁-C₆alkoxy; or phenyl, which is unsubstituted or substituted by one or more halogens.

Compounds of formula I may be prepared by reacting a compound of formula II

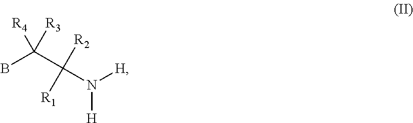

in which B, R₁, R₂, R₃ and R₄ are as defined under formula I; with a compound of formula IIIA

A-C(═O)—R* (IIIA),

in which A is as defined under formula I, and R* is halogen, hydroxy or C₁ alkoxy, preferably chloro, in the presence of a base, such as triethylamine, Hunig base, sodium bicarbonate, sodium carbonate, potassium carbonate, pyridine or quinoline, but preferably triethylamine, and in a solvent, such as diethylether, TBME, THF, dichloromethane, chloroform, DMF or NMP, for between 10 minutes and 48 hours, preferably 12 to 24 hours, and between 0° C. and reflux, preferably 20 to 25° C.

When R* is hydroxy, a coupling agent, such as benzotriazol-1-yloxytris(dimethylamino) phosphoniumhexafluorophosphate, bis-(2-oxo-3-oxazolidinyl)-phosphinic acid chloride (BOP-Cl), N,N′-dicyclohexylcarbodiimide (DCC) or 1,1′-carbonyl-diimidazole (CDI), may be used.

Some of the intermediates of the formula II

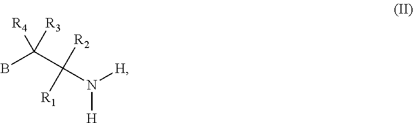

in which B, R₁, R₂, R₃ and R₄ are as defined under formula I, are novel and were developed specifically for the preparation of the compounds of the formula I. Accordingly, these intermediates of the formula II also form part of the subject-matter of the present invention. In a preferred group of compounds of the formula II, R₃ is halogen and B, R₁, R₂ and R₄ are as defined under formula I.

In another preferred group of compounds of the formula II, R₁ and R₂ together are a C₂-C₅alkylene group and B, R₃ and R₄ are as defined under formula I.

Intermediates of the formula II, in which B, R₁, R₂, R₃ and R₄ are as defined under formula I; may be prepared according to the following reaction schemes (schemes 1 to 11) or in analogy to those reaction schemes.

Intermediates of formula IIb

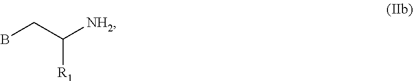

in which B is as defined under formula I and R₁ is hydrogen or C₁-C₆alkyl, C₃-C₆cycloalkyl, C₂-C₆alkenyl or C₂-C₆alkynyl, all of which are unsubstituted or substituted by one or more substituents R₅ (intermediates of formula II, in which R₂, R₃ and R₄ are hydrogen) may be prepared by reaction scheme 1.

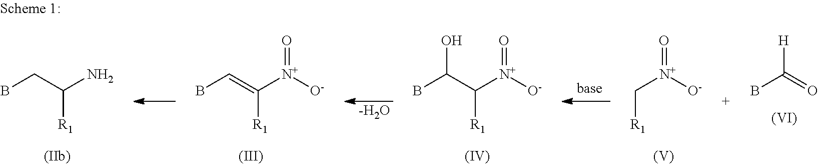

Nitroalkenes of formula III, in which B and R₁ are as defined under formula IIb, can be prepared by a Henry-reaction (nitroaldol-reaction) of a nitroalkane of formula V, in which R₁ is as defined under formula IIb, with a carbonyl compound of formula (VI), in which B is as defined under formula IIB, according to (a) Baer, H. H., Urbas, L. The chemistry of the nitro and nitroso groups; Feuer, H., Ed.; Interscience: New York, 1970; Vol. 2, pp 75-20; (b) Schickh, G.; Apel, H. G. Methoden der Organischen Chemie (Houben-Weyl Stuttgart, 1971; Vol. 10/1, pp 9-462; (c) Kabalka, G. W.; Varma, R.s. Org. Prep. Proc. Int. 1987, 283-328; or (d) Luzzio, F. A. Tetrahedron 2001, 57, 915-945; followed by a dehydration step of the resultant 2-nitro alcohol intermediates of formula IV, in which which B and R₁ are as defined under formula IIb. Such a dehydration step is described, for example, in Org. Synthesis Coll Vol I, 413, (1941). The mentioned reactions are carried out at temperatures of between 0-80° C. in convenient protic and aprotic solvents, but also under solvent-free conditions. Convienient bases described in the literature include alkali metal hydroxides, alkaline earth oxides, carbonates, bicarbonates, alkoxides and quarternary ammonium salts.

Reduction of the nitroalkenes III may be accomplished using lithium aluminium hydride in an ether solvent such as diethyl ether, ethylene glycol dimethyl ether, diethylene glycol dimethyl ether, tetrahydrofuran or dioxane, or by catalytic reduction over Raney nickel or a noble metal catalyst. The reduction is carried out at temperatures of between 20-80° C.

Intermediates of formula IIc

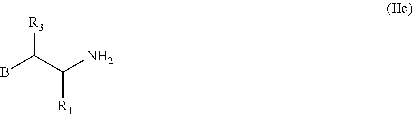

in which B is as defined under formula I, R₁ is hydrogen or C₁-C₆alkyl, C₃-C₆cycloalkyl, C₂-C₆alkenyl or C₂-C₆alkynyl, all of which are unsubstituted or substituted by one or more substituents R₅ and R₃ is C₁-C₆alkyl, C₃-C₆cycloalkyl, C₂-C₆alkenyl or C₂-C₆alkynyl, all of which are unsubstituted or substituted by one or more substituents R₅ and intermediates of formula IId

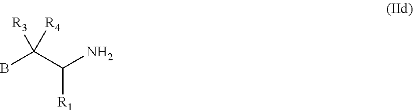

in which B is as defined under formula I, R₁ is hydrogen or C₁-C₆alkyl, C₃-C₆cycloalkyl, C₂-C₆alkenyl or C₂-C₆alkynyl, all of which are unsubstituted or substituted by one or more substituents R₅ and R₃ and R₄ independently from each other are C₁-C₆alkyl, C₃-C₆cycloalkyl, C₂-C₆alkenyl or C₂-C₆alkynyl, all of which are unsubstituted or substituted by one or more substituents R₅ may be prepared by reaction scheme 2.

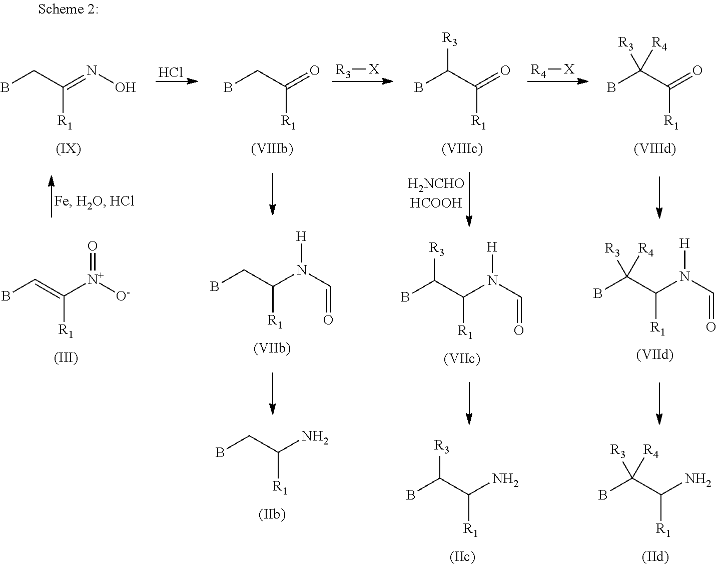

The nitroalkenes of formula III, in which B and R₁ are as defined under formula IIb, which can be prepared according to scheme 1, may be reduced with iron and hydrochloric acid to give oximes of formula IX, in which B and R₁ are as defined under formula IIb. Said oximes can be hydrolyzed to ketones of formula VIIIb, in which B and R₁ are as defined under formula IIb, as it is described, for example, in M. Kulka and H. Hibbert J. Am. Chem. Soc. 65, 1180 (1943) and in Prasun K. Pradhan et al. Synthetic Commun., 35, 913-922, 2005. The reaction is carried out at temperatures of between 40-100° C. in a convenient organic solvent such as methanol, ethanol, tert-butanol, trifluoroethanol or dioxane. The alkylation of the ketone of formula VIIIb with a compound R₃—X, in which R₃ is as defined under formula IIc and X is a leaving group, such as halogen, mesylate or tosylate, in the presence of a base yields an α-alkylated ketone of formula VIIIc, wherein B, R₁ and R₃ are as defined under formula IIc. Said ketones of formula VIIIc can be further alkylated with R₄—X, in which R₄ is as defined under formula IId and X is a leaving group, such as halogen, mesylate or tosylate, to give α,α-bis alkylated ketones of formula VIIId, wherein B, R₁, R₃ and R₄ are as defined under formula IIc. The reactions are advantageously carried out in aprotic inert organic solvents. Such solvents are hydrocarbons such as benzene, toluene, xylene or cyclohexane, ethers such as diethyl ether, ethylene glycol dimethyl ether, diethylene glycol dimethyl ether, tetrahydrofuran or dioxane, amides such as N,N-dimethylformamide, diethylformamide or N-methylpyrrolidinone. The reaction temperatures are between −20° C. and +120° C. Suitable bases are inorganic bases such as hydrides, e.g. sodium hydride or calcium hydride, hydroxides, e.g. sodium hydroxide or potassium hydroxide, carbonates such as sodium carbonate and potassium carbonate, or hydrogen carbonates such as potassium hydrogen carbonate and sodium hydrogen carbonate may also be used as bases. The bases can be used as such or else with catalytic amounts of a phase-transfer catalyst, for example a crown ether, in particular 18-crown-6, or a tetraalkylammonium salt. The reductive Leuckart amination reaction of the ketones of formula VIIIb, VIIIc and VIIId with formamide in the presence of formic acid produces the N-formyl-2-arylethylamines of formulae VIIb, VIIc and VIId, wherein B, R₁, R₃ and R₄ are as defined under formula IId. The reaction temperatures are advantageously between 120° C. and 220° C. Cp*Rh(III) complex catalysts like [RhCp*Cl₂]₂ catalyses the reductive amination of ketones using formamide at 50-70° C. see Masato Kitamura et al. J. Org. Chem. 2002, 67, 8685-8687.

Said N-formyl-2-arylethylamines of formulae VIIb, VIIc and VIId can be hydrolyzed to the amines of formula IIb, IIc and IId under acidic (conc. HCl) or basic (10% aq. NaOH) conditions at reflux temperatures.

Intermediates of formula IIe

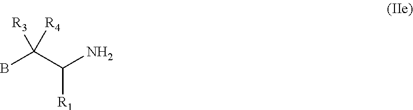

in which B is as defined under formula I, R₁ is C₁-C₆alkyl, C₃-C₆cycloalkyl, C₂-C₆alkenyl or C₂-C₆alkynyl, all of which are unsubstituted or substituted by one or more substituents R₅, R₄ is C₁-C₆alkyl, C₃-C₆cycloalkyl, C₂-C₆alkenyl or C₂-C₆alkynyl, all of which are unsubstituted or substituted by one or more substituents R₅, and R₃ is hydrogen, halogen, C₁-C₆alkyl, C₃-C₆cycloalkyl, C₂-C₆alkenyl or C₂-C₆alkynyl, all of which are unsubstituted or substituted by one or more substituents R₅, may be prepared by reaction scheme 3.

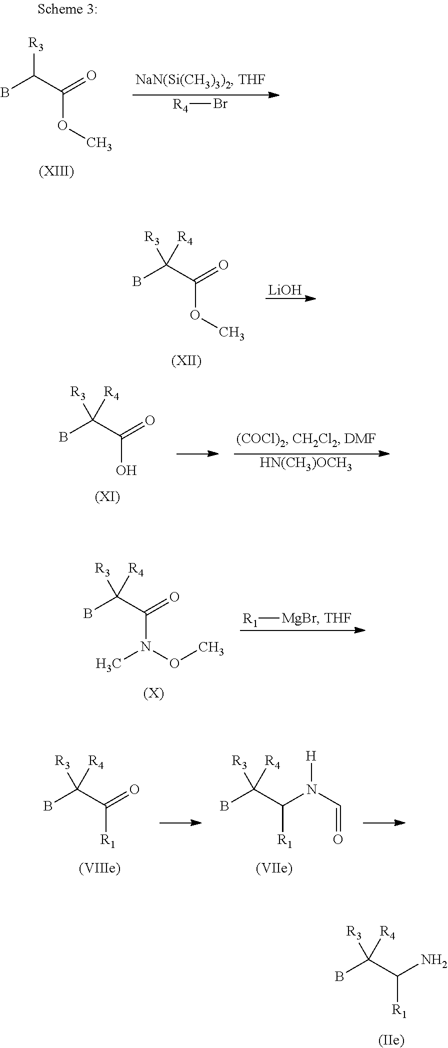

Ketones of the formula VIIIIe, wherein B, R₁, R₃ and R₄ are as defined under formula IIe, can be synthesized by the alkylation of an arylacetate derivative of formula XIII, wherein B and

R₃ are as defined under formula IIe, with an halide, such as R₄—Br, wherein R₄ is as defined under formula IIe, to afford α,α-bis alkylated arylacetates of formula XII, wherein B, R₃ and R₄ are as defined under formula IIe. The compound of formula XII is hydrolyzed by an hydroxide, such as LiOH. The resultant acid of the formula XI, wherein B, R₃ and R₄ are as defined under formula IIe, can then be converted to the corresponding acylchloride and this acylchloride can then in situ be reacted with N,O-dimethylhydroxylamine to afford a Weinreb amide of formula X, wherein B, R₃ and R₄ are as defined under formula IIe. A subsequent reaction with a Grignard reagent of the formula R₁-MgBr, wherein R₁ is as defined under formula IIe, yields the ketone of formula VIIIIe, which can be converted to a compound of formula IIe by reactions as described in scheme 2.

Intermediates of formula IIf

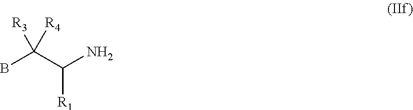

in which B, R₁, R₃ and R₄ are as defined under formula I, may be prepared by reaction scheme 4, 5 or 6.

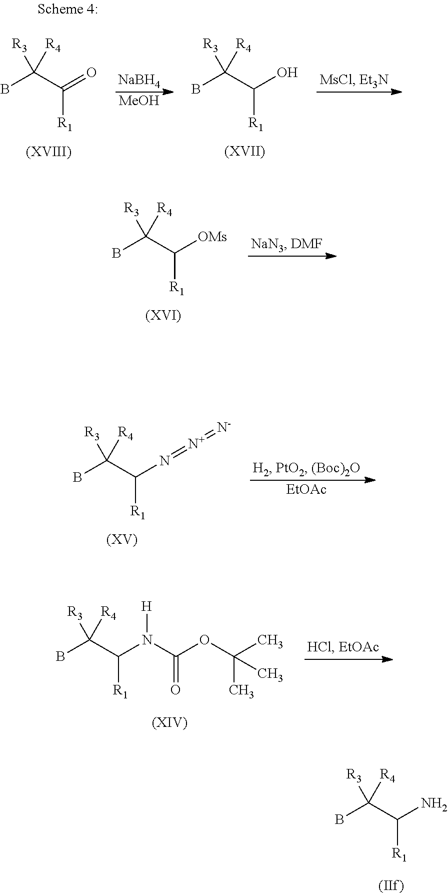

Ketones of the formula XVIII, in which B, R₁, R₃ and R₄ are as defined under formula I, can be reduced with borohydride to yield alcohols of formula XVII, in which B, R₁, R₃ and R₄ are as defined under formula I. Reaction of these alcohols with methanesulfonyl chloride affords the mesylates of formula XVI, in which B, R₁, R₃ and R₄ are as defined under formula I, which are reacted with sodium azide to form azides of formula XV, in which B, R₁, R₃ and R₄ are as defined under formula I. Reduction of these azides in the presence of hydrogen, a metal catalyst, and Boc-anhydride affords the acylated amines of formula XIV, in which B, R₁, R₃ and R₄ are as defined under formula I. The Boc groups can be conveniently removed in the presence of a strong acid, such as HCl, to yield the amines of formula IIf.

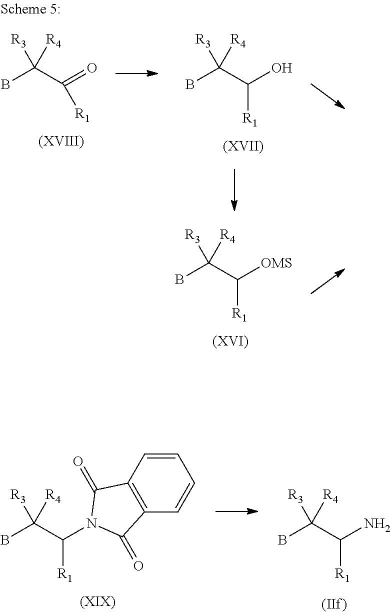

The phthalimides of formula XIX, in which B, R₁, R₃ and R₄ are as defined under formula I, can be synthesized directly from an alcohol of formula XVII, in which B, R₁, R₃ and R₄ are as defined under formula I, under Mitsunobu-conditions, or via a mesylate of formula XVI, in which B, R₁, R₃ and R₄ are as defined under formula I. The phthalimides of formula XIX can then be cleaved to the corresponding amines of formula IIf. The alcohols of formula XVII can be prepared from ketones of formula VIII as described in scheme 4.

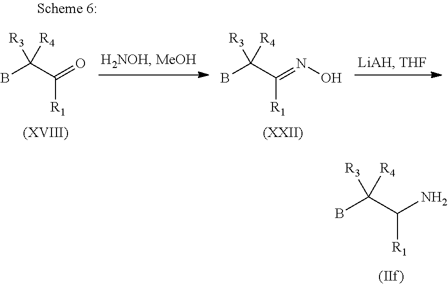

The ketones of formula XVIII can be reacted with hydroxylamine to form the oximes of formula XXII, in which B, R₁, R₃ and R₄ are as defined under formula I, which can then be reduced with lithium aluminium hydride to yield the amines of formula IIf.

Intermediates of formula IIg

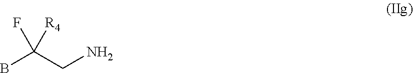

in which B and R₄ are as defined under formula I, may be prepared by reaction scheme 7.

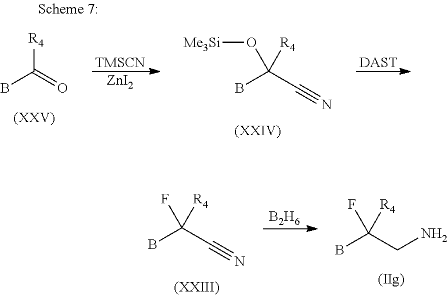

2-fluorophenylacetonitriles of formula XXIII, in which B and R₄ are as defined under formula I, can be converted to the corresponding 2-fluoro-2-phenethylamines of formula IIg. The intermediates of formula XXIII can be prepared from carbonyl compounds of formula XXV, in which B and R₄ are as defined under formula I, by way of the corresponding cyanohydrin trimethylsilylethers of formula XXIV, in which B and R₄ are as defined under formula I, by treating the corresponding cyanohydrin trimethylsilylethers of formula XXIV with diethylaminosulfur trifluoride (DAST) in dichloromethane as it is for example described in Tetrahedron Letters, Vol. 25, No. 46, pp 5227-5230, 1984.

Intermediates of formula IIh

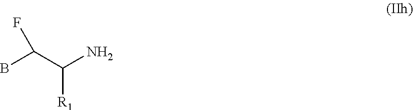

in which B and R₁ are as defined under formula I, may be prepared according to reaction schemes 8, 9 or 10.

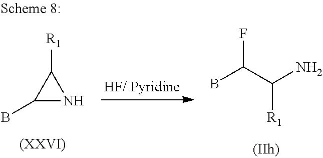

Aziridines of formula XXVI, in which B and R₁ are as defined under formula I, undergo ring-opening by Olah's reagent to give the amines of formula IIh; the reaction conditions are described for example in Tetrahedron Letters, No. 35, pp 3247-3250 1978.

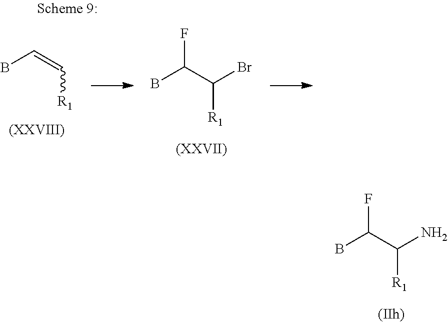

Halofluorination of alkenes of formula XXVIII, in which B and R₁ are as defined under formula I, in the presence of triethylamine tris-hydrofluoride yield the corresponding intermediates of formula XXVII, in which B and R₁ are as defined under formula I. Said intermediates of formula XXVII can be used as precursors of amines of formula IIh by using synthesis methods known to the skilled person.

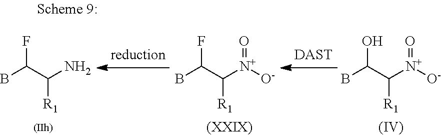

2-nitro alcohols of formula IV, as described in Scheme 1 in which B and R₁ are as defined under formula I can be treated with DAST in dichloromethane at room temperature to prepare the fluoro-nitro compound of formula XXIX, in which B and R₁ are as defined under formula I, which can be reduced under standard reaction conditions to the compounds of formula IIh.

Intermediates of formula IIi

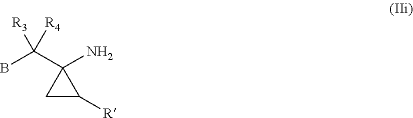

in which B, R₃ and R₄ are as defined under formula I and R′ is hydrogen or C₁-C₆alkyl, may be prepared according to reaction scheme 11.

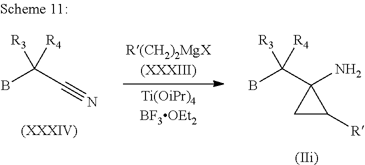

Nitriles of formula XXXIV, in which B, R₃ and R₄ are as defined under formula I, undergo a Ti-(II)-mediated coupling with Grignard reagents of formula XXXIII, in which R′ is hydrogen or C₁-C₆alkyl, to afford the cyclopropylamines of formula IIi. Reaction conditions for this reaction are described, for example, by P. Bertus, J. Szymoniak, J. Org. Chem. 2002, 67, 3965-3968 and in EP-1-595-873.

For preparing all further compounds of the formula I functionalized according to the definitions of A, B, R₁, R₂, R₃ and R₄, there are a large number of suitable known standard methods, such as alkylation, halogenation, acylation, amidation, oximation, oxidation and reduction. The choice of the preparation methods which are suitable are depending on the properties (reactivity) of the substituents in the intermediates.

The compounds of the formula IIIA are known and some of them are commercially available. They can be prepared analogously as described, for example, in WO 00/09482, WO 02/38542, WO 04/018438, EP-0-589-301, WO 93/11117 and Arch. Pharm. Res. 2000, 23(4), 315-323.

Some of the compounds of formula II are known and are commercially available or can be prepared according to the above-mentioned references or according to methods known in the art.

The compounds of formula V, V₁, R₃—X, R₄—X, XII₁, R₁-MgBr, R₄—Br, XVIII, XXV, XXVI, XXVIII, XXXI, XXXII, XXXIII and XXXIV are known and are commercially available or can be prepared according to the above-mentioned references or according to methods known in the art.

The reactions leading to compounds of the formula I are advantageously carried out in aprotic inert organic solvents. Such solvents are hydrocarbons such as benzene, toluene, xylene or cyclohexane, chlorinated hydrocarbons such as dichloromethane, trichloromethane, tetrachloromethane or chlorobenzene, ethers such as diethyl ether, ethylene glycol dimethyl ether, diethylene glycol dimethyl ether, tetrahydrofuran or dioxane, nitriles such as acetonitrile or propionitrile, amides such as N,N-dimethylformamide, diethylformamide or N-methylpyrrolidinone. The reaction temperatures are advantageously between −20° C. and +120° C. In general, the reactions are slightly exothermic and, as a rule, they can be carried out at room temperature. To shorten the reaction time, or else to start the reaction, the mixture may be heated briefly to the boiling point of the reaction mixture. The reaction times can also be shortened by adding a few drops of base as reaction catalyst. Suitable bases are, in particular, tertiary amines such as trimethylamine, triethylamine, quinuclidine, 1,4-diazabicyclo[2.2.2]octane, 1,5-diazabicyclo[4.3.0]non-5-ene or 1,5-diazabicyclo[5.4.0]undec-7-ene. However, inorganic bases such as hydrides, e.g. sodium hydride or calcium hydride, hydroxides, e.g. sodium hydroxide or potassium hydroxide, carbonates such as sodium carbonate and potassium carbonate, or hydrogen carbonates such as potassium hydrogen carbonate and sodium hydrogen carbonate may also be used as bases. The bases can be used as such or else with catalytic amounts of a phase-transfer catalyst, for example a crown ether, in particular 18-crown-6, or a tetraalkylammonium salt.

The compounds of formula I can be isolated in the customary manner by concentrating and/or by evaporating the solvent and purified by recrystallization or trituration of the solid residue in solvents in which they are not readily soluble, such as ethers, aromatic hydrocarbons or chlorinated hydrocarbons.

The compounds I and, where appropriate, the tautomers thereof, can be present in the form of one of the isomers which are possible or as a mixture of these, for example in the form of pure isomers, such as antipodes and/or diastereomers, or as isomer mixtures, such as enantiomer mixtures, for example racemates, diastereomer mixtures or racemate mixtures, depending on the number, absolute and relative configuration of asymmetric carbon atoms which occur in the molecule and/or depending on the configuration of non-aromatic double bonds which occur in the molecule; the invention relates to the pure isomers and also to all isomer mixtures which are possible and is to be understood in each case in this sense hereinabove and hereinbelow, even when stereochemical details are not mentioned specifically in each case.

Diastereo-isomeric mixtures or racemate mixtures of compounds I, which can be obtained depending on which starting materials and procedures have been chosen can be separated in a known manner into the pure diasteromers or racemates on the basis of the physicochemical differences of the components, for example by fractional crystallization, distillation and/or chromatography.

Enantiomeric mixtures, such as racemates, which can be obtained in a similar manner can be resolved into the optical antipodes by known methods, for example by recrystallization from an optically active solvent, by chromatography on chiral adsorbents, for example high-performance liquid chromatography (HPLC) on acetyl celulose, with the aid of suitable mi-croorganisms, by cleavage with specific, immobilized enzymes, via the formation of inclusion compounds, for example using chiral crown ethers, where only one enantiomer is complexed, or by conversion into diastereomeric salts, for example by reacting a basic end-pro-duct racemate with an optically active acid, such as a carboxylic acid, for example camphor, tartaric or malic acid, or sulfonic acid, for example camphorsulfonic acid, and separating the diastereomer mixture which can be obtained in this manner, for example by fractional cry-stallization based on their differing solubilities, to give the diastereomers, from which the de-sired enantiomer can be set free by the action of suitable agents, for example basic agents.

Pure diastereomers or enantiomers can be obtained according to the invention not only by separating suitable isomer mixtures, but also by generally known methods of diastereose-lective or enantioselective synthesis, for example by carrying out the process according to the invention with starting materials of a suitable stereochemistry.

It is advantageous to isolate or synthesize in each case the biologically more effective iso-mer, for example enantiomer or diastereomer, or isomer mixture, for example enantiomer mixture or diastereomer mixture, if the individual components have a different biological activity.

The compounds I and, where appropriate, the tautomers thereof, can, if appropriate, also be obtained in the form of hydrates and/or include other solvents, for example those which may have been used for the crystallization of compounds which are present in solid form.

It has now been found that the compounds of formula I according to the invention have, for practical purposes, a very advantageous spectrum of activities for protecting useful plants against diseases that are caused by phytopathogenic microorganisams, such as fungi, bacteria or viruses.

The invention relates to a method of controlling or preventing infestation of useful plants by phytopathogenic microorganisms, wherein a compound of formula I is applied as active ingredient to the plants, to parts thereof or the locus thereof. The compounds of formula I according to the invention are distinguished by excellent activity at low rates of application, by being well tolerated by plants and by being environmentally safe. They have very useful curative, preventive and systemic properties and are used for protecting numerous useful plants. The compounds of formula I can be used to inhibit or destroy the diseases that occur on plants or parts of plants (fruit, blossoms, leaves, stems, tubers, roots) of different crops of useful plants, while at the same time protecting also those parts of the plants that grow later e.g. from phytopathogenic microorganisms.

It is also possible to use compounds of formula I as dressing agents for the treatment of plant propagation material, in particular of seeds (fruit, tubers, grains) and plant cuttings (e.g. rice), for the protection against fungal infections as well as against phytopathogenic fungi occurring in the soil.

Furthermore the compounds of formula I according to the invention may be used for controlling fungi in related areas, for example in the protection of technical materials, including wood and wood related technical products, in food storage or in hygiene management.

The compounds of formula I are, for example, effective against the phytopathogenic fungi of the following classes: Fungi imperfecti (e.g. Botrytis, Pyricularia, Helminthosporium, Fusarium, Septoria, Cercospora and Alternaria) and Basidiomycetes (e.g. Rhizoctonia, Hemileia, Puccinia). Additionally, they are also effective against the Ascomycetes classes (e.g. Venturia and Erysiphe, Podosphaera, Monilinia, Uncinula) and of the Oomycetes classes (e.g. Phytophthora, Pythium, Plasmopara). Outstanding activity has been observed against powdery mildew diseases (Uncinula necator). Furthermore, the novel compounds of formula I are effective against phytopathogenic bacteria and viruses (e.g. against Xanthomonas spp, Pseudomonas spp, Erwinia amylovora as well as against the tobacco mosaic virus). Good activity has been observed against Asian soybean rust (Phakopsora pachyrhizi).

Within the scope of the invention, useful plants to be protected typically comprise the following species of plants: cereal (wheat, barley, rye, oat, rice, maize, sorghum and related species); beet (sugar beet and fodder beet); pomes, drupes and soft fruit (apples, pears, plums, peaches, almonds, cherries, strawberries, raspberries and blackberries); leguminous plants (beans, lentils, peas, soybeans); oil plants (rape, mustard, poppy, olives, sunflowers, coconut, castor oil plants, cocoa beans, groundnuts); cucumber plants (pumpkins, cucum-bers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocado, cinnamomum, camphor) or plants such as tobacco, nuts, coffee, eggplants, sugar cane, tea, pepper, vines, hops, bananas and natural rubber plants, as well as ornamentals.

The term “useful plants” is to be understood as including also useful plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors or PPO (protoporphyrinogen-oxidase) inhibitors) as a result of conventional methods of breeding or genetic engineering. An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady®, Herculex I® and LibertyLink®.

The term “useful plants” is to be understood as including also useful plants which have been so transformed by the use of recombinant DNA techniques that they are capable of synthesising one or more selectively acting toxins, such as are known, for example, from toxin-producing bacteria, especially those of the genus Bacillus.

The term “useful plants” is to be understood as including also useful plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

The term “locus” of a useful plant as used herein is intended to embrace the place on which the useful plants are growing, where the plant propagation materials of the useful plants are sown or where the plant propagation materials of the useful plants will be placed into the soil. An example for such a locus is a field, on which crop plants are growing.

The term “plant propagation material” is understood to denote generative parts of the plant, such as seeds, which can be used for the multiplication of the latter, and vegetative material, such as cuttings or tubers, for example potatoes. There may be mentioned for example seeds (in the strict sense), roots, fruits, tubers, bulbs, rhizomes and parts of plants.

Germinated plants and young plants which are to be transplanted after germination or after emergence from the soil, may also be mentioned. These young plants may be protected before transplantation by a total or partial treatment by immersion. Preferably “plant propagation material” is understood to denote seeds.

The compounds of formula I can be used in unmodified form or, preferably, together with carriers and adjuvants conventionally employed in the art of formulation.

Therefore the invention also relates to compositions for controlling and protecting against phytopathogenic microorganisms, comprising a compound of formula I and an inert carrier, and to a method of controlling or preventing infestation of useful plants by phytopathogenic microorganisms, wherein a composition, comprising a compound of formula I as active ingredient and an inert carrier, is applied to the plants, to parts thereof or the locus thereof.

To this end compounds of formula I and inert carriers are conveniently formulated in known manner to emulsifiable concentrates, coatable pastes, directly sprayable or dilutable solutions, dilute emulsions, wettable powders, soluble powders, dusts, granulates, and also encapsulations e.g. in polymeric substances. As with the type of the compositions, the methods of application, such as spraying, atomising, dusting, scattering, coating or pouring, are chosen in accordance with the intended objectives and the prevailing circumstances. The compositions may also contain further adjuvants such as stabilizers, antifoams, viscosity regulators, binders or tackifiers as well as fertilizers, micronutrient donors or other formulations for obtaining special effects.

Suitable carriers and adjuvants can be solid or liquid and are substances useful in formula-tion technology, e.g. natural or regenerated mineral substances, solvents, dispersants, wetting agents, tackifiers, thickeners, binders or fertilizers. Such carriers are for example described in WO 97/33890.

The compounds of formula I or compositions, comprising a compound of formula I as active ingredient and an inert carrier, can be applied to the locus of the plant or plant to be treated, simultaneously or in succession with further compounds. These further compounds can be e.g. fertilizers or micronutrient donors or other preparations which influence the growth of plants. They can also be selective herbicides as well as insecticides, fungicides, bactericides, nematicides, molluscicides or mixtures of several of these preparations, if desired together with further carriers, surfactants or application promoting adjuvants customarily employed in the art of formulation.

A preferred method of applying a compound of formula I, or a composition, comprising a compound of formula I as active ingredient and an inert carrier, is foliar application. The frequency of application and the rate of application will depend on the risk of infestation by the corresponding pathogen. However, the compounds of formula I can also penetrate the plant through the roots via the soil (systemic action) by drenching the locus of the plant with a liquid formulation, or by applying the compounds in solid form to the soil, e.g. in granular form (soil application). In crops of water rice such granulates can be applied to the flooded rice field. The compounds of formula I may also be applied to seeds (coating) by impregna-ting the seeds or tubers either with a liquid formulation of the fungicide or coating them with a solid formulation.

A formulation, i.e. a composition comprising the compound of formula I and, if desired, a solid or liquid adjuvant, is prepared in a known manner, typically by intimately mixing and/or grinding the compound with extenders, for example solvents, solid carriers and, optionally, surface-active compounds (surfactants).

The agrochemical formulations will usually contain from 0.1 to 99% by weight, preferably from 0.1 to 95% by weight, of the compound of formula I, 99.9 to 1% by weight, preferably 99.8 to 5% by weight, of a solid or liquid adjuvant, and from 0 to 25% by weight, preferably from 0.1 to 25% by weight, of a surfactant.

Whereas it is preferred to formulate commercial products as concentrates, the end user will normally use dilute formulations.

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient rates of application are from 10 mg to 1 g of active substance per kg of seeds. The rate of application for the desired action can be determined by experiments. It depends for example on the type of action, the developmental stage of the useful plant, and on the application (location, timing, application method) and can, owing to these parameters, vary within wide limits.

Surprisingly, it has now been found that the compounds of formula I can also be used in methods of protecting crops of useful plants against attack by phytopathogenic organisms as well as the treatment of crops of useful plants infested by phytopathogenic organisms comprising administering a combination of glyphosate and at least one compound of formula I to the plant or locus thereof, wherein the plant is resistant or sensitive to glyphosate.

Said methods may provide unexpectedly improved control of diseases compared to using the compounds of formula I in the absence of glyphosate. Said methods may be effective at enhancing the control of disease by compounds of formula I. While the mixture of glyphosate and at least one compound of formula I may increase the disease spectrum controlled, at least in part, by the compound of formula I, an increase in the activity of the compound of formula I on disease species already known to be controlled to some degree by the compound of formula I can also be the effect observed.

Said methods are particularly effective against the phytopathogenic organisms of the kingdom Fungi, phylum Basidiomycot. class Uredinomycetes, subclass Urediniomycetidae and the order Uredinales (commonly referred to as rusts). Species of rusts having a particularly large impact on agriculture include those of the family Phakopsoraceae, particularly those of the genus Phakopsora, for example Phakopsora pachyrhizi, which is also referred to as Asian soybean rust, and those of the family Pucciniaceae, particularly those of the genus Puccinia such as Puccinia graminis, also known as stem rust or black rust, which is a problem disease in cereal crops and Puccinia recondita, also known as brown rust.

An embodiment of said method is a method of protecting crops of useful plants against attack by a phytopathogenic organism and/or the treatment of crops of useful plants infested by a phytopathogenic organism, said method comprising simultaneously applying glyphosate, including salts or esters thereof, and at least one compound of formula I, which has activity against the phytopathogenic organism to at least one member selected from the group consisting of the plant, a part of the plant and the locus of the plant.

Surprisingly, it has now been found that the compounds of formula I, or a pharmaceutical salt thereof, described above have also an advantageous spectrum of activity for the treatment and/or prevention of microbial infection in an animal.

“Animal” can be any animal, for example, insect, mammal, reptile, fish, amphibian, preferably mammal, most preferably human. “Treatment” means the use on an animal which has microbial infection in order to reduce or slow or stop the increase or spread of the infection, or to reduce the infection or to cure the infection. “Prevention” means the use on an animal which has no apparent signs of microbial infection in order to prevent any future infection, or to reduce or slow the increase or spread of any future infection.

According to the present invention there is provided the use of a compound of formula I in the manufacture of a medicament for use in the treatment and/or prevention of microbial infection in an animal. There is also provided the use of a compound of formula I as a pharmaceutical agent. There is also provided the use of a compound of formula I as an antimicrobial agent in the treatment of an animal. According to the present invention there is also provided a pharmaceutical composition comprising as an active ingredient a compound of formula I, or a pharmaceutically acceptable salt thereof, and a pharmaceutically acceptable diluent or carrier. This composition can be used for the treatment and/or prevention of antimicrobial infection in an animal. This pharmaceutical composition can be in a form suitable for oral administration, such as tablet, lozenges, hard capsules, aqueous suspensions, oily suspensions, emulsions dispersible powders, dispersible granules, syrups and elixirs. Alternatively this pharmaceutical composition can be in a form suitable for topical application, such as a spray, a cream or lotion. Alternatively this pharmaceutical composition can be in a form suitable for parenteral administration, for example injection. Alternatively this pharmaceutical composition can be in inhalable form, such as an aerosol spray.

The compounds of formula I are effective against various microbial species able to cause a microbial infection in an animal. Examples of such microbial species are those causing Aspergillosis such as Aspergillus fumigatus, A. flavus, A. terrus, A. nidulans and A. niger, those causing Blastomycosis such as Blastomyces dermatitidis; those causing Candidiasis such as Candida albicans, C. glabrata, C. tropicalis, C. parapsilosis, C. krusei and C. lusitaniae; those causing Coccidioidomycosis such as Coccidioides immitis; those causing Cryptococcosis such as Cryptococcus neoformans; those causing Histoplasmosis such as Histoplasma capsulatum and those causing Zygomycosis such as Absidia corymbifera, Rhizomucor pusillus and Rhizopus arrhizus. Further examples are Fusarium Spp such as Fusarium oxysporum and Fusarium solani and Scedosporium Spp such as Scedosporium apiospermum and Scedosporium prolificans. Still further examples are Microsporum Spp, Trichophyton Spp, Epidermophyton Spp, Mucor Spp, Sporothorix Spp, Phialophora Spp, Cladosporium Spp, Petriellidium spp, Paracoccidioides Spp and Histoplasma Spp.

The following non-limiting Examples illustrate the above-described invention in greater detail without limiting it.

### PREPARATION EXAMPLES

### Example P1

### Preparation of 3-difluoromethyl-1-methyl-1H-pyrazole-4-carboxylic acid [2-(4-chlorophenyl)-ethyl]-amide (compound no. 1.001)

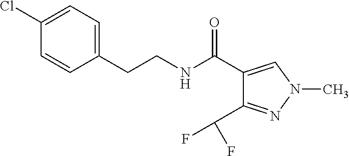

To a solution of 2-(4-chlorophenyl)-ethylamine (0.39 g, 2.5 mmol) and triethylamine (0.50 g, 5.0 mmol) in dichloromethane (10 ml) was added at 0° C. a solution of 3-difluoromethyl-1-methyl-1H-pyrazole-4-carbonyl chloride (0.49 g, 2.5 mmol) in dichloromethane (5 ml) and stirred for one hour. Dichloromethane (40 ml) and water (20 ml) was added and the layers were separated. The aqueous layer was extracted with dichloromethane (20 ml). The combined organic layers were washed with 1N NaOH (15 ml), 1N HCl (15 ml), (10% sodium chloride solution (15 ml), dried over Na₂SO₄ and concentrated in vacuo to give a raw material which was purified by flash chromatography over silicagel (eluent: hexane/ethylacetate 1:1) to afford 0.71 g (90% of theory) of 3-difluoromethyl-1-methyl-1H-pyrazole-4-carbxylic acid [2-(4-chlorophenyl)-ethyl]-amide (compound no. 1.001) in the form of a colourless oil.

¹H NMR (400 MHz, CDCl₃): δ 2.86 (t, 2H, CH₂), 3.64 (q, 2H, CH₂), 3.84 (s, 3H, NCH₃), 6.40 (t, 1H, NH), 6.79 (t, 1H, CHF₂, J=54 Hz), 7.14 (d, 2H, Ar—H), 7.23 (d, 2H, Ar—H), 7.85 (s, 1H, pyrazole-H).

MS [M+H]⁺ 314/316.

### Example P2

### Preparation of 3-difluoromethyl-1-methyl-1H-pyrazole-4-carboxylic acid [2-(2,4-dichlorophenyl)-1-methyl-ethyl]-amide (compound no. 1.197)

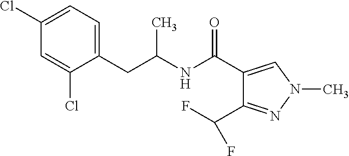

A solution of 3-difluoromethyl-1-methyl-1H-pyrazole-4-carbonyl chloride (1.95 g; 10 mmol) in dichloromethane (10 ml) was added dropwise to a stirred solution of 2.04 g (10 mmol) 2-(2,4-dichloro-phenyl)-1-methyl-ethylamine (compound Z1.197), which was prepared as described in example P9, and triethylamine (0.152 g; 15 mmol) in dichloromethane (30 ml). The reaction mixture was stirred for 1 hr at ambient temperature then allowed to stand for 3 h. The reaction mixture was washed with 1M NaOH (20 ml) and with 1M HCl (20 ml) and then dried over Na₂SO₄. After removal of the solvent the residue was purified by flash chromatography over silica gel (eluant: hexane/ethyl acetate 1:1). 2.21 g (61% of theory) of 3-difluoromethyl-1-methyl-1H-pyrazole-4-carboxylic acid [2-(2,4-dichlorophenyl)-1-methyl-ethyl]amide (compound no. 1.197) was obtained in the form of a solid (m.p. 157° C.).

¹H NMR (400 MHz, CDCl₃): δ 1.24 (d, 3H, CH₃), 2.95 (m, 2H, CH₂), 3.90 (s, 3H, NCH₃), 4.46 (m, 1H, CH), 6.21 (t, 1H, NH), 6.80 (t, 1H, CHF₂), 7.14-7.19 (m, 2H, Ar—H), 7.37 (d, 1H, Ar—H), 7.84 (s, 1H, pyrazole-H).

MS [M+H]⁺ 362/364/366.

### Example P3

### Preparation of 3-difluoromethyl-1-methyl-1H-pyrazole-4-carboxylic acid [2-(2,4-dichlorophenyl)-2-fluoro-ethyl]amide (compound no. 1.206)

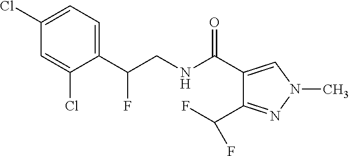

A solution of 3-difluoromethyl-1-methyl-1H-pyrazole-4-carbonyl chloride (0.148 g; 0.758 mmol) in dichloromethane (3 ml) was added dropwise to a stirred solution of 0.150 g (0.721 mmol) 2-(2,4-dichloro-phenyl)-2-fluoro-propylamine hydrochloride (compound Z1.206), which was prepared as described in example P10, and triethylamine (301 μl; 2.16 mmol) in dichloromethane (12 ml). The reaction mixture was stirred for 2 hr at ambient temperature then washed with 1M NaOH (10 ml), 1M HCl (10 ml), water (10 ml) and then dried over Na₂SO₄. 190 mg (72% of theory) of 3-difluoromethyl-1-methyl-1H-pyrazole-4-carboxylic acid [2-(2,4-dichlorophenyl)-2-fluoro-ethyl]-amide (compound no. 1.206) was obained in form of a resin.

¹H NMR (400 MHz, CDCl₃): δ 3.62-3.75 and 3.92-4.15 (m, 2H, CH₂), 3.87 (s, 3H, NCH₃), 5.86-5.89 and 5.98-6.01 (m, 1H, CH), 6.67 (t, 1H, NH), 6.82 (t, 1H, CHF₂), 7.29 (d, 1H, Ar—H), 7.37 (d, 1H, Ar—H), 7.41 (d, 1H, Ar—H), 7.91 (s, 1H, pyrazole-H).

MS [M+H]⁺ 366/368/370.

### Example P4

### Preparation of 3-difluoromethyl-1-methyl-1H-pyrazole-4-carboxylic acid [2-(2,4-dichlorophenyl)-2-fluoro-1-methyl-ethyl]amide (compound no. 1.216)

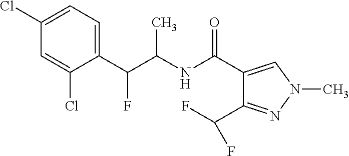

A solution of 3-difluoromethyl-1-methyl-1H-pyrazole-4-carbonyl chloride (0.098 g; 0.50 mmol) in dichloromethane (1 ml) was added dropwise to a stirred solution of 0.129 g (0.50 mmol) 2-(2,4-dichloro-phenyl)-2-fluoro-1-methyl-ethylamine hydrochloride (compound Z1.216), which was prepared as described in example P11 and triethylamine (0.202; 2.0 mmol) in dichloromethane (3 ml). The reaction mixture was stirred for 2 hr at ambient temperature. After removal of the solvent the residue was purified by flash chromatography over silica gel (eluent: cyclo hexane/ethyl acetate 1:1). 0.15 g (78.9% of theory) of 3-difluoromethyl-1-methyl-1H-pyrazole-4-carboxylic acid [2-(2,4-dichlorophenyl)-2-fluoro-1-methyl-ethyl]amide (compound no. 1.216) was obtained in form of a resin.

¹H NMR (400 MHz, CDCl₃): δ 1.43 (d, 3H, CH₃), 3.87 (s, 3H, NCH₃), 4.69-4.80 (m, 1H, CH), 5.73 and 5.84 (d, 1H, CH), 6.51 (t, 1H, NH), 6.79 (t, 1H, CHF₂), 7.19 (d, 1H, Ar—H), 7.35-7.37 (m, 2H, Ar—H), 7.79 (s, 1H, pyrazole-H).

MS [M+H]⁺ 380/382/384.

### Example P5

### Preparation of 3-difluoromethyl-1-methyl-1H-pyrazole-4-carboxylic acid [2-(2,4-dichlorophenyl)-2-fluoro-propyl]-amide (compound no. 1.221)

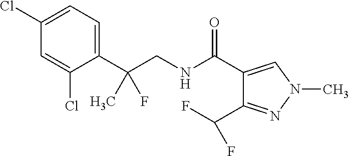

A solution of 3-difluoromethyl-1-methyl-1H-pyrazole-4-carbonyl chloride (0.158 g; 0.813 mmol) in dichloromethane (3 ml) was added dropwise to a stirred solution of 0.2 g (0.774 mmol) 2-(2,4-dichloro-phenyl)-2-fluoro-propylamine hydrochloride (compound Z1.221), which was prepared as described in example P12, and triethylamine (323 μl; 2.32 mmol) in dichloromethane (12 ml). The reaction mixture was stirred for 3 hr at ambient temperature then washed with 1M NaOH (10 ml), 1M HCl (10 ml), water (10 ml) and then dried over Na₂SO₄. 190 mg (65% of theory) of 3-difluoromethyl-1-methyl-1H-pyrazole-4-carboxylic acid [2-(2,4-dichlorophenyl)-2-fluoro-propyl]-amide (compound no. 1.221) was obtained in form of a resin.

¹H NMR (400 MHz, CDCl₃): δ 1.77 and 1.87 (s, 3H, CH₃), 3.95 (s, 3H, NCH₃), 4.12-4.14 and 4.20-4.22 (q, 2H, CH₂), 6.52 (t, 1H, NH), 6.73 (t, 1H, CHF₂), 7.28 (m, 1H, Ar—H), 7.39 (d, 1H, Ar—H), 7.40 (d, 1H, Ar—H), 7.86 (s, 1H, pyrazole-H).

MS [M+H]⁺ 380/382/384.

### Example P6

### Preparation of 3-difluoromethyl-1-methyl-1H-pyrazole-4-carboxylic acid [1-(2,4-dichlorobenzyl)-cyclopropyl]-amide (compound no. 1.231)

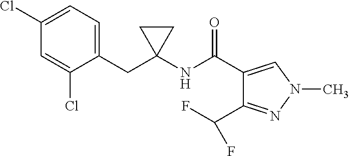

A solution of 3-difluoromethyl-1-methyl-1H-pyrazole-4-carbonyl chloride (0.190 g; 0.98 mmol) in dichloromethane (3 ml) was added dropwise to a stirred solution of 0.2 g (0.93 mmol) 1-(2,4-dichloro-benzyl)-cyclopropylamine (compound Z1.231), which was prepared as described in example P13, and triethylamine (0.22 ml; 1.50 mmol) in dichloromethane (7 ml). The reaction mixture was stirred for 2 hr at ambient temperature then washed with 1M NaOH (5 ml), 1M HCl (5 ml), brine (10 ml) and then dried over Na₂SO₄. The raw material was then purified by flash chromatography over silicagel (eluent: hexane/ethylacetate 1:1). 145 mg (40% of theory) of 3-difluoromethyl-1-methyl-1H-pyrazole-4-carboxylic acid [1-(2,4-dichlorobenzyl)-cyclopropyl]-amide (compound no. 1.231) was obtained in form of a solid (m.p. 165-168° C.).

¹H NMR (400 MHz, CDCl₃): δ 0.88-0.99 (m, 4H, 2×CH₂), 3.18 (s, 3H, CH₃), 3.86 (s, 3H, NCH₃), 6.45 (t, 1H, NH), 6.76 (t, 1H, CHF₂), 7.13 (m, 1H, Ar—H), 7.22 (d, 1H, Ar—H), 7.40 (d, 1H, Ar—H), 7.86 (s, 1H, pyrazole-H).

MS [M+H]⁺ 374/376/378.

### Example P7

### Preparation of 3-difluoromethyl-1-methyl-1H-pyrazole-4-carboxylic acid [2-(4-bromo-2-chlorophenyl)-1-methyl-ethyl]-amide (compound no. 1.451)

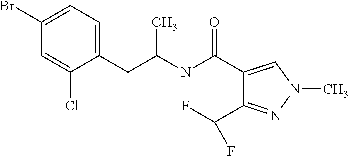

A mixture of 2-(4-bromo-2-chloro-phenyl)-1-methyl-ethylamine (compound Z1.451), which was prepared as described in example P14 (3.36 g, 13 mmol), 3-difluoromethyl-1-methyl-1H-pyrazole-4-carboxylic acid (2.16 g, 12 mmol) and 10 ml pyridine was cooled under nitrogen atmosphere to 0° C. Phosphorus oxychloride (2.08 g, 13 mmol) was slowly added. The mixture was stirred at 80° C. for 12 h, diluted with water and extracted with ethyl acetate. The ethyl acetate phase was washed with 1.5 N HCl, 10% NaOH, water and brine and dried over sodium sulphate. After removal of the solvent the residue was washed with hexane. 3.25 g (59% of theory) of 3-difluoromethyl-1-methyl-1H-pyrazole-4-carboxylic acid [2-(4-bromo-2-chloro-phenyl)-1-methyl-ethyl]amide (compound no. 1.451) was obtained in the form of a light brown solid (purity: 97%).

1HNMR—(400 MHz, CDCl3): 1.25 δ (d, 3H), 2.95 δ (ddd, 2H, CH2), 3.9 δ (s, 3H, NCH3), 4.45 δ (m, 1H, CHN), 6.2 δ (s, 1H, NH), 6.79 δ (t, 1H, CHF2), 7.2 δ (d, 1H), 7.3 δ (d, 1H), 7.5 δ (s, 1H), 7.84 δ (S, 1H, pyrazole-H),

MS [M+H]⁺ 406/408/4108

### Example P8

### Preparation of 3-difluoromethyl-1-methyl-1H-pyrazole-4-carboxylic acid [2-(3,4′-dichlorobiphenyl-4-yl)-1-methyl-ethyl]-amide (compound no. 1.462)

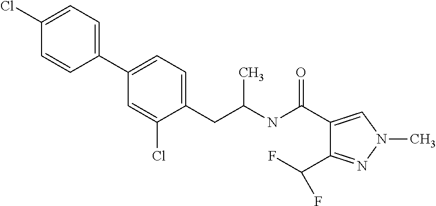

Anhydrous potassium carbonate (0.25 g, 0.02 mmol) and palladium acetate (0.007 g, 0.031 mmol) were added to a solution of 3-difluoromethyl-1-methyl-1H-pyrazole-4-carboxylic acid [2-(4-bromo-2-chloro-phenyl)-1-methyl-ethyl]amide (compound Z1.451), prepared as described in example P7 (0.25 g, 0.62 mmol), in 20 ml ethanol/water (ethanol/water=3:1) under a nitrogen atmosphere. 4-Chlorobenzene boronic acid (0.105 g, 0.677 mmol) was added. The reaction mixture was stirred for 16 h. The reaction was monitored using HPLC. When the reaction was completed, the reaction mixture was filtered over a celite bed. The filtrate was concentrated and purified by chromatography using a silica column (60-120μ mesh) and hexane:ethylacetate (25%) as eluent. 163 mg (60% of theory) of 3-difluoromethyl-1-methyl-1H-pyrazole-4-carboxylic acid [2-(3,4′-dichlorobiphenyl-4-yl)-1-methyl-ethyl]-amide (compound no. 1.462) was obtained in the form of a solid (m.p. 96-98° C., purity: 93%).

1HNMR—(400 MHz, CDCl3): 1.25 δ (d, 3H), 2.96 δ (ddd, 2H, CH2), 3.83 δ (s, 3H, NCH3), 4.4 δ (m, 1H, CHN), 6.1 δ (s, 1H, NH), 6.75 δ (t, 1H, CHF2), 7.05-7.49 δ (m, 7H—Ar), 7.8 δ (S, 1H, pyrazole-H),

MS [M+H]⁺ 438/440

### Example P9

### Preparation of 2-(2,4-dichlorophenyl)-1-methyl-ethylamine hydrochloride (compound no. Z1.197)

### a) Preparation of 2,4-dichloro-1-((E)-2-nitro-propenyl)-benzene

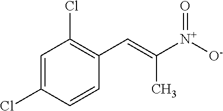

In a sulfonation flask 2,4-dichloro-benzaldehyde (77 g, 0.44 mol), nitroethane (216 ml, 3.04 mol) and ammonium acetate (81.4 g, 1.06 mol) were added to glacial acetic acid (600 ml). The resulting solution was heated to 90° C. for three hours. After removal of the solvent ice-water (400 ml) was added. The solid product was collected by filtration, washed with water and recrystallized from ethanol. 55.9 g (55% of theory) of 2,4-dichloro-1-((E)-2-nitro-propenyl)-benzene was obtained in the form of a yellow solid (m.p. 79-81° C.).

¹H NMR (400 MHz, CDCl₃): δ 8.11 (s, 1H), 7.51 (d, 1H), 7.34 (dd, 1H), 7.27 (d, 1H), 2.33 (s, 3H, CH₃).

### b) Preparation of 2-(2,4-dichlorophenyl)-1-methyl-ethylamine hydrochloride (compound no. Z1.197)

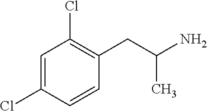

To a stirred suspension of lithium aluminium hydride (3 equiv, 30 mmol, 1.14 g) in dry tetrahydrofurane (30 ml) under nitrogen atmosphere was added dropwise a solution of 2,4-dichloro-1-((E)-2-nitro-propenyl)-benzene (10 mmol, 2.32 g) in dry THF (20 ml) under cooling with an ice bath. After stirring for 10 minutes the suspension was heated to reflux for 1 hour, then the mixture was cooled to 0° C. and excess lithium aluminium hydride was decomposed by the sequential dropwise addition of water (40 ml), tert.-butylmethylether (20 ml), 20% NaOH (20 ml) and water (40 ml) under stirring. The reaction product was collected by filtration and washed with MTBE. The filtrate was washed with brine, dried over MgSO₄, filtered and dried under reduced pressure. 2.0 g (98% of theory) of 2-(2,4-dichlorophenyl)-1-methyl-ethylamine (compound Z1.197) was obtained in the form of a brown oil.

MS [M+H]⁺ 204/206/208.

The 2-(2,4-dichlorophenyl)-1-methyl-ethylamine was used in example P2 without further purification.

### Example P10

### Preparation of 2-(2,4-dichlorophenyl)-2-fluoro-ethylamine hydrochloride (compound no. Z1.206)

### a) Preparation of (2,4-dichloro-phenyl)-fluoro-acetonitrile

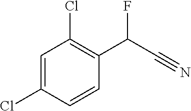

To a stirred suspension of ZnI₂ (160 mg, 0.5 mmol), 2,4-dichloro-benzaldehyde (40 g, 228 mmol) and dichloromethane (15 ml) under nitrogen atmosphere was added dropwise trimethylsilylcyanide (29 ml, 228 mmol) under cooling to 5° C. The reaction mixture was stirred at ambient temperature for 20 minutes. The reaction mixture was diluted with dry dichloromethane (250 ml), cooled to 5° C., and a solution of DAST (33 ml, 250 mmol) in dichloromethane (50 ml) was added dropwise. The reaction mixture was stirred for 30 minutes at ambient temperature, then ice-water was added (700 ml). Dichloromethane (250 ml) was added and the organic layer was extracted. The organic layer was washed sequentially with water (250 ml), 0.5N HCl (200 ml), saturated NaHCO₃ (200 ml), and water (200 ml). The organic layer was dried over NaSO₄, filtered, and concentrated. The concentrated liquid was further purified by flash chromatography over silicagel (eluent: hexane/ethylacetate 9:1). 38.9 g (35.4% of theory) of (2,4-dichloro-phenyl)-fluoro-acetonitrile was obtained in the form of a liquid.

¹H NMR (400 MHz, CDCl₃): δ 7.64 (d, 1H), 7.49 (d, 1H), 7.41 (dd, 1H), 6.37 (d, 1H, J=44 Hz).

### b) Preparation of 2-(2,4-dichlorophenyl)-2-fluoro-ethylamine hydrochloride (compound no. Z1.206)

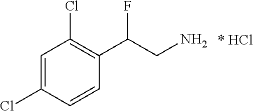

(2,4-Dichlorophenyl)-fluoro-acetonitrile (1.0 g, 4.9 mmol) in anhydrous tetrahydrofurane (10 ml) was cooled to 0° C. 1 M borane-THF (19.6 ml, 19.6 mmol) was added dropwise and the reaction mixture was stirred at 0° C. for 1 hour. After this ethanol (25 ml) was added dropwise, then the reaction mixture was acidified with ethanolic HCl and concentrated in vacuo. The residue was triturated with ether. 2-(2,4-Dichlorophenyl)-2-fluoro-ethylamine hydrochloride was obtained in the form of a white solid.

MS [M+H]⁺ 208/210/212.

### Example P11

### Preparation of 2-(2,4-dichlorophenyl)-2-fluoro-1-methyl-ethylamine hydrochloride (compound no. Z1.216):

### a) Preparation of 1-(2,4-dichlorophenyl)-2-nitro-propan-1-ol

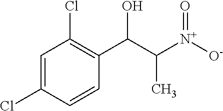

To a stirred solution of nitroethane (8.3 g, 0.11 mol) in acetonitrile (150 ml) was added anhydrous potassium phosphate (1.0 g, 4.6 mmol) followed by 2,4-dichloro-benzaldehyde (17.5 g, 0.10 mol). The reaction mixture was stirred for 4 hours. Water (300 ml) was added and the reaction mixture was extracted with diethyl ether (200 ml). The organic extract was washed with water and dried over anhydrous Na₂SO₄, the solvent was removed and the resulting residue was purified by flash chromatography over silicagel (eluent: cyclohexane/ethylacetate 9:1). 20.7 g (82.5% of theory) of a threo/erythro-mixture of 1-(2,4-dichloro-phenyl)-2-nitro-propan-1-ol was obtained. Crystallisation from cyclohexane yielded pure erythro 1-(2,4-dichlorophenyl)-2-nitro-propan-1-ol.

(erythro-form) ¹H NMR (400 MHz, CDCl₃): δ 1.43 (d, 3H, CH₃), 2.92 (d, 1H₂OH), 4.84 (m, 1H, CH), 5.79 (t, 1H, CH), 7.34 (d, 1H, Ar—H), 7.40 (d, 1H, Ar—H), 7.59 (d, 1H, Ar—H).

### b) Preparation of 2,4-dichloro-1-(1-fluoro-2-nitro-propyl)benzene

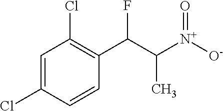

To a stirred mixture of erythro 1-(2,4-dichlorophenyl)-2-nitro-propan-1-ol (2.5 g, 10.0 mmol) in dry dichloromethane (20 ml) under nitrogen atmosphere DAST (1.3 ml, 10.0 mmol) in dichloromethane (5 ml) was added dropwise under cooling to 5° C. The solution was stirred at ambient temperature for 1 hour. Dichloromethane (80 ml) was added and the organic layer was washed sequentially with saturated NaHCO₃ (50 ml), 1M HCl (30 ml) and sole (30 ml). The organic layer was dried over NaSO4, filtered, and concentrated. 2.5 g of 2,4-dichloro-1-(1-fluoro-2-nitro-propyl)-benzene was obtained in the form of a brown oil.

### c) Preparation of 2-(2,4-dichlorophenyl)-2-fluoro-1-methyl-ethylamine hydrochloride (compound no. Z1.216)

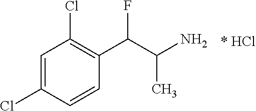

2,4-dichloro-1-(1-fluoro-2-nitro-propyl)-benzene (0.67 g, 2.64 mmol), prepared as described above, was dissolved without further purification in iso-propanol (52 ml). 1M HCl (26.4 ml, 26.4 mmol) was added. Zinc (3.46 g, 52.8 mmol) was added in small portions and the suspension was stirred for 2 hours at ambient temperature. A saturated solution of NaHCO₃ (80 ml) was added, the mixture was stirred for 15 minutes and then filtered through a small plug of Celite and washed with ethylacetate. The organic layer was dried over NaSO₄, filtered, concentrated under reduced pressure, diethylether and then ethanolic HCl (0.1 ml) was added dropwise. The mixture was then concentrated under reduced pressure. The residue was triturated with ether, yielding the required hydrochloride in the form of a white solid. 0.175 g (25.6% of theory) of erythro 2-(2,4-dichlorophenyl)-2-fluoro-1-methyl-ethylamine hydrochloride (compound no. Z1.216) was obtained in form of a white solid.

MS [M+H]⁺ 222/224/226.

### Example P12

### Preparation of 2-(2,4-dichlorophenyl)-2-fluoro-propylamine hydrochloride (compound no. Z1.221

### a) Preparation of 2-(2,4-dichloro-phenyl)-2-fluoro-propionitrile

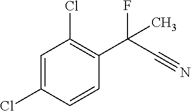

To a stirred mixture of ZnI₂ (20 mg, 0.06 mmol) and 2,4-dichloro-acetophenone (4.3 g, 22.8 mmol) under nitrogen atmosphere trimethylsilylcyanide (2.9 ml, 22.8 mmol) was added dropwise at 5° C. The solution was stirred at ambient temperature for 20 minutes. Dry dichloromethane (20 ml) was added and the solution was cooled to 5° C. Then a solution of diethylamino sulfurtrifluoride DAST (3.3 ml, 25.0 mmol) in dichloromethane (5 ml) was added dropwise. The solution was stirred for 30 minutes at ambient temperature and then ice-water (70 ml) was added. Dichloromethane (25 ml) was added and the organic layer was separated from the aqueous layer. The organic layer was washed sequentially with water (25 ml), 0.5N HCl (25 ml), saturated NaHCO₃ (25 ml), and water (20 ml). The organic layer was dried over NaSO₄, filtered, concentrated under reduced pressure and purified by flash chromatography over silicagel (eluent: hexane/ethylacetate 9:1). 3.14 g (63% of theory) of 2-(2,4-dichloro-phenyl)-2-fluoro-propionitrile was obtained in the form of a liquid.

¹H NMR (400 MHz, CDCl₃): δ 7.52 (d, 1H), 7.48 (d, 1H), 7.37 (dd, 1H), 2.15 (d, 3H, J=24 Hz).

### b) Preparation of 2-(2,4-dichlorophenyl)-2-fluoro-propylamine hydrochloride (compound no. Z1.221)

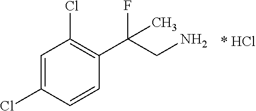

To a mixture of 2-(2,4-Dichloro-phenyl)-2-fluoro-propionitrile (1.0 g, 4.9 mmol) in anhydrous THF (10 ml) 1 M borane-THF (19.6 ml, 19.6 mmol) was added dropwise at 0° C. The reaction mixture was stirred in an ice bath for 1 hour. Ethanol (25 ml) was added dropwise and the mixture was acidified with ethanolic HCl and concentrated in vacuo. The residue was triturated with ether and 820 mg (64.5% of theory) of 2-(2,4-dichlorophenyl)-2-fluoro-propylamine hydrochloride ws obtained in the form of a white solid (m.p. 152-155° C.).

¹H NMR (400 MHz, DMSO): δ 8.44 (sbr, 2H), 7.72 (d, 1H), 7.65 (d, 1H), 7.53 (dd, 1H), 3.55 (m, 2H), 1.85 (d, 3H, J=28 Hz).

MS [M+H]⁺ 222/224/226.

### Example P13

### Preparation of 1-(2,4-dichloro-benzyl)-cyclopropylamine (compound no. 21.231)

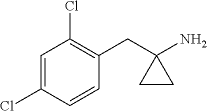

To a solution of (2,4-dichloro-phenyl)-acetonitrile (6.3 g, 33 mmol) and Ti(OiPr)₄ (36.3 mmol) in ether (150 ml) EtMgBr (1 M in ether, 66 ml, 66 mmol) was added dropwise at ambient temperature. The reaction mixture was stirred for 1 hour and BF₃.Et₂O (66 mmol) was added, the reaction mixture was further stirred for 30 minutes at ambient temperature. 1N NaOH (120 ml, 120 mmol) was added and the organic layer was separated. The aqueous layer was extracted with ether and the organic phases were combined. After washing with brine (100 ml), the organic layer was dried over sodium sulphate, the solvent was removed and the obtained product was purified by flash chromatography over silicagel (eluent: dichloromethane/methanol 9:1). 3.1 g (43% of theory) of 1-(2,4-dichloro-benzyl)-cyclopropylamine was obtained in the form of a liquid.

MS [M+H]⁺ 216/218/220.

### Example P14

### Preparation of 2-(4-bromo-2-chlorophenyl)-1-methyl-ethylamine (compound no. Z1.451)

### a) Preparation of 4-bromo-2-chloro-1-dibromomethyl-benzene

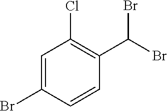

A mixture of 4-bromo-2-chlorotoluene (10 g, 48.6 mmol), N-bromosuccinimide (43.3 g, 243.3 mmol), benzoyl peroxide (0.5 g) and CCl₄ (80 ml) was heated to reflux for 6 h. Completion of the reaction was confirmed by TLC. After cooling a yellow precipitate was isolated by filtration and washed with CCl₄. The organic layer was concentrated and both the concentreated organic layer and the precipitate were purified by chromatography using silica column (60-120μ mesh) and hexane as eluent. 17.5 g (98% of theory) of 4-bromo-2-chloro-1-dibromomethyl-benzene was obtained.

¹HNMR (400 MHz, CDCl₃): −7.02 δ (s, 1H), 7.5 δ (dd, 2H), 7.87 δ (d, 1H, CHBr2),

### b) Preparation of 4-bromo-2-chlorobenzaldehyde

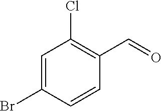

A solution of AgNO₃ (82 g, 482 mmol) in 55 ml water was added dropwise to a solution of 4-bromo-2-chloro-1-dibromomethyl-benzene (17.5 g, 48.2 mmol) in 25 ml ethanol at reflux temperature. A precipitate (AgBr) formed immediately. Heating was continued for 1 h. The reaction mixture was cooled and 200 ml water were added. The precipitate was removed by filtration and the aqueous phase was extracted with chloroform. The organic phase was washed with water and brine and dried over sodium sulphate. After removal of the solvent, 9.6 g (85% of theory) of 4-bromo-2-chlorobenzaldehyde (purity: 97%) was obtained.

¹HNMR (400 MHz, CDCl₃): 7.24 δ (td, 1H), 7.6 δ (d, 1H), 7.8 δ (d, 1H), 10.4 δ (s, 1H, CHO),

MS [M+H]⁺ 217/219/220

### c) Preparation of 4-bromo-2-chloro-1-((E)-2-nitro-propenyl)-benzene

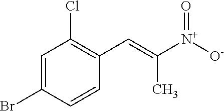

A mixture of 4-bromo-2-chlorobenzaldehyde (9.6 g, 43.8 mmol), ammonium acetate (8.44 g, 109.6 mmol) and acetic acid (25 ml) was stirred at 0° C. for 10 minutes. Nitroethane (21.6 ml, 302.4 mmol) was added slowly. The reaction mixture was heated to 110° C. for 30 minutes under a nitrogen atmosphere. Completion of reaction was confirmed by TLC. The reaction mixture was cooled to ambient temperature and ice-water was added. The aqueous solution was extracted with ethyl acetate. The organic phase was washed with water and brine and dried over sodium sulphate. The solvent was removed and the residue was purified by column chromatography using silica column (60-120μ mesh) and 2% of ethyl acetate:hexane as eluent. 6.14 g (50% of theory) of 4-bromo-2-chloro-1-((E)-2-nitro-propenyl)-benzene (purity: 98%) was obtained.

¹HNMR (400 MHz, CDCl₃): 2.3 δ (d, 3H), 7.2 δ (d, 1H), 7.5 δ (dd, 1H), 7.65 δ (d, 1H), 8 δ (s, 1H).

### d) Preparation of 4-bromo-2-chloro-1-(2-nitro-propyl)-benzene

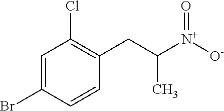

A solution of 4-bromo-2-chloro-1-((E)-2-nitro-propenyl)-benzene (6.1 g, 22.1 mmol) in 40 ml methanol was cooled to 0° C. under a nitrogen atmosphere. Sodium borohydride (2.52 g, 66.3 mmol) was added slowly. The reaction mixture was stirred at ambient temperature for 6 h and ethyl acetate was added. After removal of the solvent the residue was dissolved in water and extracted with ethyl acetate. The organic phase was washed with water and brine and dried over sodium sulphate. The solvent was evaporated and 5.5 g (89% of theory) of 4-bromo-2-chloro-1-(2-nitro-propyl)-benzene were obtained (purity: 86%).

¹HNMR (400 MHz, CDCl₃): 1.5 δ (d, 3H), 3.15 δ (dd, 1H, CHH), 3.35 δ (dd, 1H, CHH), 4.85 δ (m, CHN), 7.05 δ (d, 1H), 7.3 δ (dd, 1H), 7.5 δ (d, 1H)

MS [M+H]⁺ (C₉H₉BrClNO₂) (248/249/250)

### e) Preparation of 2-(4-bromo-2-chlorophenyl)-1-methyl-ethylamine (compound no. Z1.451)

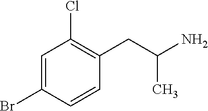

4-bromo-2-chloro-1-(2-nitro-propyl)-benzene (6.8 g, 24 mmol) was dissolved in 1:1 methanol/water (40 ml). Iron powder (4 g, 72 mmol) and NH₄Cl (7.8 g, 144 mmol) were added. The reaction mixture was heated to 65° C. for 12 h. The reaction mixture was filtered over a celite bed and washed with methanol. The volume of the filtrate was reduced and water was added. The obtained aqueous solution was acidified using 2 N HCl and washed with diethyl ether. The pH of the aqueous phase was increased over 7 by addition of 10% NaOH. The aqueous phase was extracted with ethyl acetate. The organic phase was dried over sodium sulphate and the solvent was removed. 3.6 g (59% of theory) of 2-(4-bromo-2-chlorophenyl)-1-methyl-ethylamine (compound Z1.451) was obtained in the form of a brown oil. Compound Z1.451 was used in example P7 without further purification.

1HNMR— (400 MHz, CDCl3): 1.25 δ (d, 3H), 2.95 δ (dd, 2H, CH2), 3.2 δ (m, 1H, CHN), 4.42 δ (m, NH2), 7.3 δ (d, 1H), 7.5 δ (dd, 1H), 7.7 δ (d, 1H),

MS [M+H]⁺ 248/249/250

### Tables 1 to 7: Compounds of Formula IA

The invention is further illustrated by the preferred individual compounds of formula (IA) listed below in Tables 1 to 7. Characterising data is given in Table 14.

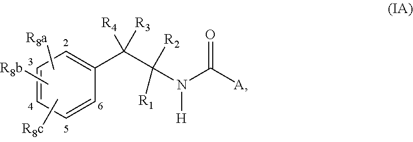

Each of Tables 1 to 7, which follow the Table Y below, comprises 482 compounds of the formula (IA) in which R₁, R₂, R₃, R₄, R₈ₐ, R_{8b} and R_{8c} have the values given in Table Y and A has the value given in the relevant Table 1 to 7. Thus Table 1 corresponds to Table Y when Y is 1 and A has the value given under the Table 1 heading, Table 2 corresponds to Table Y when Y is 2 and A has the value given under the Table 2 heading, and so on for Tables 3 to 7.

TABLE Y

| Comp. | | | | | | | |
|---|---|---|---|---|---|---|---|
| No. | R₁ | R₂ | R₃ | R₄ | R₈ₐ | R_{8b} | R_{8c} |
| Y.001 | H | H | H | H | 4-Cl | H | H |
| Y.002 | CH₃ | H | H | H | 4-Cl | H | H |
| Y.003 | CH₂CH₃ | H | H | H | 4-Cl | H | H |
| Y.004 | (CH₂)₂CH₃ | H | H | H | 4-Cl | H | H |
| Y.005 | CH(CH₃)₂ | H | H | H | 4-Cl | H | H |
| Y.006 | F | H | H | H | 4-Cl | H | H |
| Y.007 | H | H | CH₃ | H | 4-Cl | H | H |
| Y.008 | H | H | CH₂CH₃ | H | 4-Cl | H | H |
| Y.009 | H | H | (CH₂)₂CH₃ | H | 4-Cl | H | H |
| Y.010 | H | H | CH(CH₃)₂ | H | 4-Cl | H | H |
| Y.011 | H | H | F | H | 4-Cl | H | H |
| Y.012 | CH₃ | CH₃ | H | H | 4-Cl | H | H |
| Y.013 | CH₂CH₃ | CH₃ | H | H | 4-Cl | H | H |
| Y.014 | (CH₂)₂CH₃ | CH₃ | H | H | 4-Cl | H | H |
| Y.015 | CH(CH₃)₂ | CH₃ | H | H | 4-Cl | H | H |
| Y.016 | F | CH₃ | H | H | 4-Cl | H | H |
| Y.017 | CH₃ | H | CH₃ | H | 4-Cl | H | H |
| Y.018 | CH₂CH₃ | H | CH₃ | H | 4-Cl | H | H |
| Y.019 | (CH₂)₂CH₃ | H | CH₃ | H | 4-Cl | H | H |
| Y.020 | CH(CH₃)₂ | H | CH₃ | H | 4-Cl | H | H |
| Y.021 | CH₃ | H | F | H | 4-Cl | H | H |
| Y.022 | CH₂CH₃ | H | F | H | 4-Cl | H | H |
| Y.023 | H | H | CH₃ | CH₃ | 4-Cl | H | H |
| Y.024 | H | H | CH₂CH₃ | CH₃ | 4-Cl | H | H |
| Y.025 | H | H | CH(CH₃)₂ | CH₃ | 4-Cl | H | H |
| Y.026 | H | H | CH₃ | F | 4-Cl | H | H |
| Y.027 | H | H | CH₂CH₃ | F | 4-Cl | H | H |
| Y.028 | H | H | (CH₂)₂CH₃ | F | 4-Cl | H | H |
| Y.029 | H | H | CH(CH₃)₂ | F | 4-Cl | H | H |
| Y.030 | H | H | F | F | 4-Cl | H | H |
| Y.031 | CH₃ | H | CH₃ | F | 4-Cl | H | H |
| Y.032 | CH₃ | H | CH₂CH₃ | F | 4-Cl | H | H |
| Y.033 | CH₃ | H | (CH₂)₂CH₃ | F | 4-Cl | H | H |
| Y.034 | CH₃ | H | CH(CH₃)₂ | F | 4-Cl | H | H |
| Y.035 | CH₃ | H | F | F | 4-Cl | H | H |
| Y.036 | (CH₂)₂ | | H | H | 4-Cl | H | H |
| Y.037 | (CH₂)₂ | | CH₃ | H | 4-Cl | H | H |
| Y.038 | (CH₂)₂ | | (CH₂)₂CH₃ | H | 4-Cl | H | H |
| Y.039 | (CH₂)₂ | | CH(CH₃)₂ | H | 4-Cl | H | H |
| Y.040 | H | H | H | H | 4-CF₃ | H | H |
| Y.041 | CH₃ | H | H | H | 4-CF₃ | H | H |
| Y.042 | CH₂CH₃ | H | H | H | 4-CF₃ | H | H |
| Y.043 | (CH₂)₂CH₃ | H | H | H | 4-CF₃ | H | H |
| Y.044 | CH(CH₃)₂ | H | H | H | 4-CF₃ | H | H |
| Y.045 | F | H | H | H | 4-CF₃ | H | H |
| Y.046 | H | H | CH₃ | H | 4-CF₃ | H | H |
| Y.047 | H | H | CH₂CH₃ | H | 4-CF₃ | H | H |
| Y.048 | H | H | (CH₂)₂CH₃ | H | 4-CF₃ | H | H |
| Y.049 | H | H | CH(CH₃)₂ | H | 4-CF₃ | H | H |
| Y.050 | H | H | F | H | 4-CF₃ | H | H |
| Y.051 | CH₃ | CH₃ | H | H | 4-CF₃ | H | H |
| Y.052 | CH₂CH₃ | CH₃ | H | H | 4-CF₃ | H | H |
| Y.053 | (CH₂)₂CH₃ | CH₃ | H | H | 4-CF₃ | H | H |
| Y.054 | CH(CH₃)₂ | CH₃ | H | H | 4-CF₃ | H | H |
| Y.055 | F | CH₃ | H | H | 4-CF₃ | H | H |
| Y.056 | CH₃ | H | CH₃ | H | 4-CF₃ | H | H |
| Y.057 | CH₂CH₃ | H | CH₃ | H | 4-CF₃ | H | H |
| Y.058 | (CH₂)₂CH₃ | H | CH₃ | H | 4-CF₃ | H | H |
| Y.059 | CH(CH₃)₂ | H | CH₃ | H | 4-CF₃ | H | H |
| Y.060 | CH₃ | H | F | H | 4-CF₃ | H | H |
| Y.061 | CH₂CH₃ | H | F | H | 4-CF₃ | H | H |
| Y.062 | H | H | CH₃ | CH₃ | 4-CF₃ | H | H |
| Y.063 | H | H | CH₂CH₃ | CH₃ | 4-CF₃ | H | H |
| Y.064 | H | H | CH(CH₃)₂ | CH₃ | 4-CF₃ | H | H |
| Y.065 | H | H | CH₃ | F | 4-CF₃ | H | H |
| Y.066 | H | H | CH₂CH₃ | F | 4-CF₃ | H | H |
| Y.067 | H | H | (CH₂)₂CH₃ | F | 4-CF₃ | H | H |
| Y.068 | H | H | CH(CH₃)₂ | F | 4-CF₃ | H | H |
| Y.069 | H | H | F | F | 4-CF₃ | H | H |
| Y.070 | CH₃ | H | CH₃ | F | 4-CF₃ | H | H |
| Y.071 | CH₃ | H | CH₂CH₃ | F | 4-CF₃ | H | H |
| Y.072 | CH₃ | H | (CH₂)₂CH₃ | F | 4-CF₃ | H | H |
| Y.073 | CH₃ | H | CH(CH₃)₂ | F | 4-CF₃ | H | H |
| Y.074 | CH₃ | H | F | F | 4-CF₃ | H | H |
| Y.075 | (CH₂)₂ | | H | H | 4-CF₃ | H | H |
| Y.076 | (CH₂)₂ | | CH₃ | H | 4-CF₃ | H | H |
| Y.077 | (CH₂)₂ | | (CH₂)₂CH₃ | H | 4-CF₃ | H | H |
| Y.078 | (CH₂)₂ | | CH(CH₃)₂ | H | 4-CF₃ | H | H |
| Y.079 | H | H | H | H | 4-OCF₃ | H | H |
| Y.080 | CH₃ | H | H | H | 4-OCF₃ | H | H |
| Y.081 | CH₂CH₃ | H | H | H | 4-OCF₃ | H | H |
| Y.082 | (CH₂)₂CH₃ | H | H | H | 4-OCF₃ | H | H |
| Y.083 | CH(CH₃)₂ | H | H | H | 4-OCF₃ | H | H |
| Y.084 | F | H | H | H | 4-OCF₃ | H | H |
| Y.085 | H | H | CH₃ | H | 4-OCF₃ | H | H |
| Y.086 | H | H | CH₂CH₃ | H | 4-OCF₃ | H | H |
| Y.087 | H | H | (CH₂)₂CH₃ | H | 4-OCF₃ | H | H |
| Y.088 | H | H | CH(CH₃)₂ | H | 4-OCF₃ | H | H |
| Y.089 | H | H | F | H | 4-OCF₃ | H | H |
| Y.090 | CH₃ | CH₃ | H | H | 4-OCF₃ | H | H |
| Y.091 | CH₂CH₃ | CH₃ | H | H | 4-OCF₃ | H | H |
| Y.092 | (CH₂)₂CH₃ | CH₃ | H | H | 4-OCF₃ | H | H |
| Y.093 | CH(CH₃)₂ | CH₃ | H | H | 4-OCF₃ | H | H |
| Y.094 | F | CH₃ | H | H | 4-OCF₃ | H | H |
| Y.095 | CH₃ | H | CH₃ | H | 4-OCF₃ | H | H |
| Y.096 | CH₂CH₃ | H | CH₃ | H | 4-OCF₃ | H | H |
| Y.097 | (CH₂)₂CH₃ | H | CH₃ | H | 4-OCF₃ | H | H |
| Y.098 | CH(CH₃)₂ | H | CH₃ | H | 4-OCF₃ | H | H |
| Y.099 | CH₃ | H | F | H | 4-OCF₃ | H | H |
| Y.100 | CH₂CH₃ | H | F | H | 4-OCF₃ | H | H |
| Y.101 | H | H | CH₃ | CH₃ | 4-OCF₃ | H | H |
| Y.102 | H | H | CH₂CH₃ | CH₃ | 4-OCF₃ | H | H |
| Y.103 | H | H | CH(CH₃)₂ | CH₃ | 4-OCF₃ | H | H |
| Y.104 | H | H | CH₃ | F | 4-OCF₃ | H | H |
| Y.105 | H | H | CH₂CH₃ | F | 4-OCF₃ | H | H |
| Y.106 | H | H | (CH₂)₂CH₃ | F | 4-OCF₃ | H | H |
| Y.107 | H | H | CH(CH₃)₂ | F | 4-OCF₃ | H | H |
| Y.108 | H | H | F | F | 4-OCF₃ | H | H |
| Y.109 | CH₃ | H | CH₃ | F | 4-OCF₃ | H | H |
| Y.110 | CH₃ | H | CH₂CH₃ | F | 4-OCF₃ | H | H |
| Y.111 | CH₃ | H | (CH₂)₂CH₃ | F | 4-OCF₃ | H | H |
| Y.112 | CH₃ | H | CH(CH₃)₂ | F | 4-OCF₃ | H | H |
| Y.113 | CH₃ | H | F | F | 4-OCF₃ | H | H |
| Y.114 | (CH₂)₂ | | H | H | 4-OCF₃ | H | H |
| Y.115 | (CH₂)₂ | | CH₃ | H | 4-OCF₃ | H | H |
| Y.116 | (CH₂)₂ | | (CH₂)₂CH₃ | H | 4-OCF₃ | H | H |
| Y.117 | (CH₂)₂ | | CH(CH₃)₂ | H | 4-OCF₃ | H | H |
| Y.118 | H | H | H | H | 4-C≡CC(CH₃)₃ | H | H |
| Y.119 | CH₃ | H | H | H | 4-C≡CC(CH₃)₃ | H | H |
| Y.120 | CH₂CH₃ | H | H | H | 4-C≡CC(CH₃)₃ | H | H |
| Y.121 | (CH₂)₂CH₃ | H | H | H | 4-C≡CC(CH₃)₃ | H | H |
| Y.122 | CH(CH₃)₂ | H | H | H | 4-C≡CC(CH₃)₃ | H | H |
| Y.123 | F | H | H | H | 4-C≡CC(CH₃)₃ | H | H |
| Y.124 | H | H | CH₃ | H | 4-C≡CC(CH₃)₃ | H | H |
| Y.125 | H | H | CH₂CH₃ | H | 4-C≡CC(CH₃)₃ | H | H |
| Y.126 | H | H | (CH₂)₂CH₃ | H | 4-C≡CC(CH₃)₃ | H | H |
| Y.127 | H | H | CH(CH₃)₂ | H | 4-C≡CC(CH₃)₃ | H | H |
| Y.128 | H | H | F | H | 4-C≡CC(CH₃)₃ | H | H |
| Y.129 | CH₃ | CH₃ | H | H | 4-C≡CC(CH₃)₃ | H | H |
| Y.130 | CH₂CH₃ | CH₃ | H | H | 4-C≡CC(CH₃)₃ | H | H |
| Y.131 | (CH₂)₂CH₃ | CH₃ | H | H | 4-C≡CC(CH₃)₃ | H | H |
| Y.132 | CH(CH₃)₂ | CH₃ | H | H | 4-C≡CC(CH₃)₃ | H | H |
| Y.133 | F | CH₃ | H | H | 4-C≡CC(CH₃)₃ | H | H |
| Y.134 | CH₃ | H | CH₃ | H | 4-C≡CC(CH₃)₃ | H | H |
| Y.135 | CH₂CH₃ | H | CH₃ | H | 4-C≡CC(CH₃)₃ | H | H |
| Y.136 | (CH₂)₂CH₃ | H | CH₃ | H | 4-C≡CC(CH₃)₃ | H | H |
| Y.137 | CH(CH₃)₂ | H | CH₃ | H | 4-C≡CC(CH₃)₃ | H | H |
| Y.138 | CH₃ | H | F | H | 4-C≡CC(CH₃)₃ | H | H |
| Y.139 | CH₂CH₃ | H | F | H | 4-C≡CC(CH₃)₃ | H | H |
| Y.140 | H | H | CH₃ | CH₃ | 4-C≡CC(CH₃)₃ | H | H |
| Y.141 | H | H | CH₂CH₃ | CH₃ | 4-C≡CC(CH₃)₃ | H | H |
| Y.142 | H | H | CH(CH₃)₂ | CH₃ | 4-C≡CC(CH₃)₃ | H | H |
| Y.143 | H | H | CH₃ | F | 4-C≡CC(CH₃)₃ | H | H |
| Y.144 | H | H | CH₂CH₃ | F | 4-C≡CC(CH₃)₃ | H | H |
| Y.145 | H | H | (CH₂)₂CH₃ | F | 4-C≡CC(CH₃)₃ | H | H |
| Y.146 | H | H | CH(CH₃)₂ | F | 4-C≡CC(CH₃)₃ | H | H |
| Y.147 | H | H | F | F | 4-C≡CC(CH₃)₃ | H | H |
| Y.148 | CH₃ | H | CH₃ | F | 4-C≡CC(CH₃)₃ | H | H |
| Y.149 | CH₃ | H | CH₂CH₃ | F | 4-C≡CC(CH₃)₃ | H | H |
| Y.150 | CH₃ | H | (CH₂)₂CH₃ | F | 4-C≡CC(CH₃)₃ | H | H |
| Y.151 | CH₃ | H | CH(CH₃)₂ | F | 4-C≡CC(CH₃)₃ | H | H |
| Y.152 | CH₃ | H | F | F | 4-C≡CC(CH₃)₃ | H | H |
| Y.153 | (CH₂)₂ | | H | H | 4-C≡CC(CH₃)₃ | H | H |
| Y.154 | (CH₂)₂ | | CH₃ | H | 4-C≡CC(CH₃)₃ | H | H |
| Y.155 | (CH₂)₂ | | (CH₂)₂CH₃ | H | 4-C≡CC(CH₃)₃ | H | H |
| Y.156 | (CH₂)₂ | | CH(CH₃)₂ | H | 4-C≡CC(CH₃)₃ | H | H |
| Y.157 | H | H | H | H | 4-p-Cl-phenyl | H | H |
| Y.158 | CH₃ | H | H | H | 4-p-Cl-phenyl | H | H |
| Y.159 | CH₂CH₃ | H | H | H | 4-p-Cl-phenyl | H | H |
| Y.160 | (CH₂)₂CH₃ | H | H | H | 4-p-Cl-phenyl | H | H |
| Y.161 | CH(CH₃)₂ | H | H | H | 4-p-Cl-phenyl | H | H |
| Y.162 | F | H | H | H | 4-p-Cl-phenyl | H | H |
| Y.163 | H | H | CH₃ | H | 4-p-Cl-phenyl | H | H |
| Y.164 | H | H | CH₂CH₃ | H | 4-p-Cl-phenyl | H | H |
| Y.165 | H | H | (CH₂)₂CH₃ | H | 4-p-Cl-phenyl | H | H |
| Y.166 | H | H | CH(CH₃)₂ | H | 4-p-Cl-phenyl | H | H |
| Y.167 | H | H | F | H | 4-p-Cl-phenyl | H | H |
| Y.168 | CH₃ | CH₃ | H | H | 4-p-Cl-phenyl | H | H |
| Y.169 | CH₂CH₃ | CH₃ | H | H | 4-p-Cl-phenyl | H | H |
| Y.170 | (CH₂)₂CH₃ | CH₃ | H | H | 4-p-Cl-phenyl | H | H |
| Y.171 | CH(CH₃)₂ | CH₃ | H | H | 4-p-Cl-phenyl | H | H |
| Y.172 | F | CH₃ | H | H | 4-p-Cl-phenyl | H | H |
| Y.173 | CH₃ | H | CH₃ | H | 4-p-Cl-phenyl | H | H |
| Y.174 | CH₂CH₃ | H | CH₃ | H | 4-p-Cl-phenyl | H | H |
| Y.175 | (CH₂)₂CH₃ | H | CH₃ | H | 4-p-Cl-phenyl | H | H |
| Y.176 | CH(CH₃)₂ | H | CH₃ | H | 4-p-Cl-phenyl | H | H |
| Y.177 | CH₃ | H | F | H | 4-p-Cl-phenyl | H | H |
| Y.178 | CH₂CH₃ | H | F | H | 4-p-Cl-phenyl | H | H |
| Y.179 | H | H | CH₃ | CH₃ | 4-p-Cl-phenyl | H | H |
| Y.180 | H | H | CH₂CH₃ | CH₃ | 4-p-Cl-phenyl | H | H |
| Y.181 | H | H | CH(CH₃)₂ | CH₃ | 4-p-Cl-phenyl | H | H |
| Y.182 | H | H | CH₃ | F | 4-p-Cl-phenyl | H | H |
| Y.183 | H | H | CH₂CH₃ | F | 4-p-Cl-phenyl | H | H |
| Y.184 | H | H | (CH₂)₂CH₃ | F | 4-p-Cl-phenyl | H | H |
| Y.185 | H | H | CH(CH₃)₂ | F | 4-p-Cl-phenyl | H | H |
| Y.186 | H | H | F | F | 4-p-Cl-phenyl | H | H |
| Y.187 | CH₃ | H | CH₃ | F | 4-p-Cl-phenyl | H | H |
| Y.188 | CH₃ | H | CH₂CH₃ | F | 4-p-Cl-phenyl | H | H |
| Y.189 | CH₃ | H | (CH₂)₂CH₃ | F | 4-p-Cl-phenyl | H | H |
| Y.190 | CH₃ | H | CH(CH₃)₂ | F | 4-p-Cl-phenyl | H | H |
| Y.191 | CH₃ | H | F | F | 4-p-Cl-phenyl | H | H |
| Y.192 | (CH₂)₂ | | H | H | 4-p-Cl-phenyl | H | H |
| Y.193 | (CH₂)₂ | | CH₃ | H | 4-p-Cl-phenyl | H | H |
| Y.194 | (CH₂)₂ | | (CH₂)₂CH₃ | H | 4-p-Cl-phenyl | H | H |
| Y.195 | (CH₂)₂ | | CH(CH₃)₂ | H | 4-p-Cl-phenyl | H | H |
| Y.196 | H | H | H | H | 2-Cl | 4-Cl | H |
| Y.197 | CH₃ | H | H | H | 2-Cl | 4-Cl | H |
| Y.198 | CH₂CH₃ | H | H | H | 2-Cl | 4-Cl | H |
| Y.199 | (CH₂)₂CH₃ | H | H | H | 2-Cl | 4-Cl | H |
| Y.200 | CH(CH₃)₂ | H | H | H | 2-Cl | 4-Cl | H |
| Y.201 | F | H | H | H | 2-Cl | 4-Cl | H |
| Y.202 | H | H | CH₃ | H | 2-Cl | 4-Cl | H |
| Y.203 | H | H | CH₂CH₃ | H | 2-Cl | 4-Cl | H |
| Y.204 | H | H | (CH₂)₂CH₃ | H | 2-Cl | 4-Cl | H |
| Y.205 | H | H | CH(CH₃)₂ | H | 2-Cl | 4-Cl | H |
| Y.206 | H | H | F | H | 2-Cl | 4-Cl | H |
| Y.207 | CH₃ | CH₃ | H | H | 2-Cl | 4-Cl | H |
| Y.208 | CH₂CH₃ | CH₃ | H | H | 2-Cl | 4-Cl | H |
| Y.209 | (CH₂)₂CH₃ | CH₃ | H | H | 2-Cl | 4-Cl | H |
| Y.210 | CH(CH₃)₂ | CH₃ | H | H | 2-Cl | 4-Cl | H |
| Y.211 | F | CH₃ | H | H | 2-Cl | 4-Cl | H |
| Y.212 | CH₃ | H | CH₃ | H | 2-Cl | 4-Cl | H |
| Y.213 | CH₂CH₃ | H | CH₃ | H | 2-Cl | 4-Cl | H |
| Y.214 | (CH₂)₂CH₃ | H | CH₃ | H | 2-Cl | 4-Cl | H |
| Y.215 | CH(CH₃)₂ | H | CH₃ | H | 2-Cl | 4-Cl | H |
| Y.216 | CH₃ | H | F | H | 2-Cl | 4-Cl | H |
| Y.217 | CH₂CH₃ | H | F | H | 2-Cl | 4-Cl | H |
| Y.218 | H | H | CH₃ | CH₃ | 2-Cl | 4-Cl | H |
| Y.219 | H | H | CH₂CH₃ | CH₃ | 2-Cl | 4-Cl | H |
| Y.220 | H | H | CH(CH₃)₂ | CH₃ | 2-Cl | 4-Cl | H |
| Y.221 | H | H | CH₃ | F | 2-Cl | 4-Cl | H |
| Y.222 | H | H | CH₂CH₃ | F | 2-Cl | 4-Cl | H |
| Y.223 | H | H | (CH₂)₂CH₃ | F | 2-Cl | 4-Cl | H |
| Y.224 | H | H | CH(CH₃)₂ | F | 2-Cl | 4-Cl | H |
| Y.225 | H | H | F | F | 2-Cl | 4-Cl | H |
| Y.226 | CH₃ | H | CH₃ | F | 2-Cl | 4-Cl | H |
| Y.227 | CH₃ | H | CH₂CH₃ | F | 2-Cl | 4-Cl | H |
| Y.228 | CH₃ | H | (CH₂)₂CH₃ | F | 2-Cl | 4-Cl | H |
| Y.229 | CH₃ | H | CH(CH₃)₂ | F | 2-Cl | 4-Cl | H |
| Y.230 | CH₃ | H | F | F | 2-Cl | 4-Cl | H |
| Y.231 | (CH₂)₂ | | H | H | 2-Cl | 4-Cl | H |
| Y.232 | (CH₂)₂ | | CH₃ | H | 2-Cl | 4-Cl | H |
| Y.233 | (CH₂)₂ | | (CH₂)₂CH₃ | H | 2-Cl | 4-Cl | H |
| Y.234 | (CH₂)₂ | | CH(CH₃)₂ | H | 2-Cl | 4-Cl | H |
| Y.235 | H | H | H | H | 3-Cl | 4-Cl | H |
| Y.236 | CH₃ | H | H | H | 3-Cl | 4-Cl | H |
| Y.237 | CH₂CH₃ | H | H | H | 3-Cl | 4-Cl | H |
| Y.238 | (CH₂)₂CH₃ | H | H | H | 3-Cl | 4-Cl | H |
| Y.239 | CH(CH₃)₂ | H | H | H | 3-Cl | 4-Cl | H |
| Y.240 | F | H | H | H | 3-Cl | 4-Cl | H |
| Y.241 | H | H | CH₃ | H | 3-Cl | 4-Cl | H |
| Y.242 | H | H | CH₂CH₃ | H | 3-Cl | 4-Cl | H |
| Y.243 | H | H | (CH₂)₂CH₃ | H | 3-Cl | 4-Cl | H |
| Y.244 | H | H | CH(CH₃)₂ | H | 3-Cl | 4-Cl | H |
| Y.245 | H | H | F | H | 3-Cl | 4-Cl | H |
| Y.246 | CH₃ | CH₃ | H | H | 3-Cl | 4-Cl | H |
| Y.247 | CH₂CH₃ | CH₃ | H | H | 3-Cl | 4-Cl | H |
| Y.248 | (CH₂)₂CH₃ | CH₃ | H | H | 3-Cl | 4-Cl | H |
| Y.249 | CH(CH₃)₂ | CH₃ | H | H | 3-Cl | 4-Cl | H |
| Y.250 | F | CH₃ | H | H | 3-Cl | 4-Cl | H |
| Y.251 | CH₃ | H | CH₃ | H | 3-Cl | 4-Cl | H |
| Y.252 | CH₂CH₃ | H | CH₃ | H | 3-Cl | 4-Cl | H |
| Y.253 | (CH₂)₂CH₃ | H | CH₃ | H | 3-Cl | 4-Cl | H |
| Y.254 | CH(CH₃)₂ | H | CH₃ | H | 3-Cl | 4-Cl | H |
| Y.255 | CH₃ | H | F | H | 3-Cl | 4-Cl | H |
| Y.256 | CH₂CH₃ | H | F | H | 3-Cl | 4-Cl | H |
| Y.247 | H | H | CH₃ | CH₃ | 3-Cl | 4-Cl | H |
| Y.258 | H | H | CH₂CH₃ | CH₃ | 3-Cl | 4-Cl | H |
| Y.259 | H | H | CH(CH₃)₂ | CH₃ | 3-Cl | 4-Cl | H |
| Y.260 | H | H | CH₃ | F | 3-Cl | 4-Cl | H |
| Y.261 | H | H | CH₂CH₃ | F | 3-Cl | 4-Cl | H |
| Y.262 | H | H | (CH₂)₂CH₃ | F | 3-Cl | 4-Cl | H |
| Y.263 | H | H | CH(CH₃)₂ | F | 3-Cl | 4-Cl | H |
| Y.264 | H | H | F | F | 3-Cl | 4-Cl | H |
| Y.265 | CH₃ | H | CH₃ | F | 3-Cl | 4-Cl | H |
| Y.266 | CH₃ | H | CH₂CH₃ | F | 3-Cl | 4-Cl | H |
| Y.267 | CH₃ | H | (CH₂)₂CH₃ | F | 3-Cl | 4-Cl | H |
| Y.268 | CH₃ | H | CH(CH₃)₂ | F | 3-Cl | 4-Cl | H |
| Y.269 | CH₃ | H | F | F | 3-Cl | 4-Cl | H |
| Y.270 | (CH₂)₂ | | H | H | 3-Cl | 4-Cl | H |
| Y.271 | (CH₂)₂ | | CH₃ | H | 3-Cl | 4-Cl | H |
| Y.272 | (CH₂)₂ | | (CH₂)₂CH₃ | H | 3-Cl | 4-Cl | H |
| Y.273 | (CH₂)₂ | | CH(CH₃)₂ | H | 3-Cl | 4-Cl | H |
| Y.274 | H | H | H | H | 2-F | 4-Cl | H |
| Y.275 | CH₃ | H | H | H | 2-F | 4-Cl | H |
| Y.276 | CH₂CH₃ | H | H | H | 2-F | 4-Cl | H |
| Y.277 | (CH₂)₂CH₃ | H | H | H | 2-F | 4-Cl | H |
| Y.278 | CH(CH₃)₂ | H | H | H | 2-F | 4-Cl | H |
| Y.279 | F | H | H | H | 2-F | 4-Cl | H |
| Y.280 | H | H | CH₃ | H | 2-F | 4-Cl | H |
| Y.281 | H | H | CH₂CH₃ | H | 2-F | 4-Cl | H |
| Y.282 | H | H | (CH₂)₂CH₃ | H | 2-F | 4-Cl | H |
| Y.283 | H | H | CH(CH₃)₂ | H | 2-F | 4-Cl | H |
| Y.284 | H | H | F | H | 2-F | 4-Cl | H |
| Y.285 | CH₃ | CH₃ | H | H | 2-F | 4-Cl | H |
| Y.286 | CH₂CH₃ | CH₃ | H | H | 2-F | 4-Cl | H |
| Y.287 | (CH₂)₂CH₃ | CH₃ | H | H | 2-F | 4-Cl | H |
| Y.288 | CH(CH₃)₂ | CH₃ | H | H | 2-F | 4-Cl | H |
| Y.289 | F | CH₃ | H | H | 2-F | 4-Cl | H |
| Y.290 | CH₃ | H | CH₃ | H | 2-F | 4-Cl | H |
| Y.291 | CH₂CH₃ | H | CH₃ | H | 2-F | 4-Cl | H |
| Y.292 | (CH₂)₂CH₃ | H | CH₃ | H | 2-F | 4-Cl | H |
| Y.293 | CH(CH₃)₂ | H | CH₃ | H | 2-F | 4-Cl | H |
| Y.294 | CH₃ | H | F | H | 2-F | 4-Cl | H |
| Y.295 | CH₂CH₃ | H | F | H | 2-F | 4-Cl | H |
| Y.296 | H | H | CH₃ | CH₃ | 2-F | 4-Cl | H |
| Y.297 | H | H | CH₂CH₃ | CH₃ | 2-F | 4-Cl | H |
| Y.298 | H | H | CH(CH₃)₂ | CH₃ | 2-F | 4-Cl | H |
| Y.299 | H | H | CH₃ | F | 2-F | 4-Cl | H |
| Y.300 | H | H | CH₂CH₃ | F | 2-F | 4-Cl | H |
| Y.301 | H | H | (CH₂)₂CH₃ | F | 2-F | 4-Cl | H |
| Y.302 | H | H | CH(CH₃)₂ | F | 2-F | 4-Cl | H |
| Y.303 | H | H | F | F | 2-F | 4-Cl | H |
| Y.304 | CH₃ | H | CH₃ | F | 2-F | 4-Cl | H |
| Y.305 | CH₃ | H | CH₂CH₃ | F | 2-F | 4-Cl | H |
| Y.306 | CH₃ | H | (CH₂)₂CH₃ | F | 2-F | 4-Cl | H |
| Y.307 | CH₃ | H | CH(CH₃)₂ | F | 2-F | 4-Cl | H |
| Y.308 | CH₃ | H | F | F | 2-F | 4-Cl | H |
| Y.309 | (CH₂)₂ | | H | H | 2-F | 4-Cl | H |
| Y.310 | (CH₂)₂ | | CH₃ | H | 2-F | 4-Cl | H |
| Y.311 | (CH₂)₂ | | (CH₂)₂CH₃ | H | 2-F | 4-Cl | H |
| Y.312 | (CH₂)₂ | | CH(CH₃)₂ | H | 2-F | 4-Cl | H |
| Y.313 | H | H | H | H | 4-F | 2-Cl | H |
| Y.314 | CH₃ | H | H | H | 4-F | 2-Cl | H |
| Y.315 | CH₂CH₃ | H | H | H | 4-F | 2-Cl | H |
| Y.316 | (CH₂)₂CH₃ | H | H | H | 4-F | 2-Cl | H |
| Y.317 | CH(CH₃)₂ | H | H | H | 4-F | 2-Cl | H |
| Y.318 | F | H | H | H | 4-F | 2-Cl | H |
| Y.319 | H | H | CH₃ | H | 4-F | 2-Cl | H |
| Y.320 | H | H | CH₂CH₃ | H | 4-F | 2-Cl | H |
| Y.321 | H | H | (CH₂)₂CH₃ | H | 4-F | 2-Cl | H |
| Y.322 | H | H | CH(CH₃)₂ | H | 4-F | 2-Cl | H |
| Y.323 | H | H | F | H | 4-F | 2-Cl | H |
| Y.324 | CH₃ | CH₃ | H | H | 4-F | 2-Cl | H |
| Y.325 | CH₂CH₃ | CH₃ | H | H | 4-F | 2-Cl | H |
| Y.326 | (CH₂)₂CH₃ | CH₃ | H | H | 4-F | 2-Cl | H |
| Y.327 | CH(CH₃)₂ | CH₃ | H | H | 4-F | 2-Cl | H |
| Y.328 | F | CH₃ | H | H | 4-F | 2-Cl | H |
| Y.329 | CH₃ | H | CH₃ | H | 4-F | 2-Cl | H |
| Y.330 | CH₂CH₃ | H | CH₃ | H | 4-F | 2-Cl | H |
| Y.331 | (CH₂)₂CH₃ | H | CH₃ | H | 4-F | 2-Cl | H |
| Y.332 | CH(CH₃)₂ | H | CH₃ | H | 4-F | 2-Cl | H |
| Y.333 | CH₃ | H | F | H | 4-F | 2-Cl | H |
| Y.334 | CH₂CH₃ | H | F | H | 4-F | 2-Cl | H |
| Y.335 | H | H | CH₃ | CH₃ | 4-F | 2-Cl | H |
| Y.336 | H | H | CH₂CH₃ | CH₃ | 4-F | 2-Cl | H |
| Y.337 | H | H | CH(CH₃)₂ | CH₃ | 4-F | 2-Cl | H |
| Y.338 | H | H | CH₃ | F | 4-F | 2-Cl | H |
| Y.339 | H | H | CH₂CH₃ | F | 4-F | 2-Cl | H |
| Y.340 | H | H | (CH₂)₂CH₃ | F | 4-F | 2-Cl | H |
| Y.341 | H | H | CH(CH₃)₂ | F | 4-F | 2-Cl | H |
| Y.342 | H | H | F | F | 4-F | 2-Cl | H |
| Y.343 | CH₃ | H | CH₃ | F | 4-F | 2-Cl | H |
| Y.344 | CH₃ | H | CH₂CH₃ | F | 4-F | 2-Cl | H |
| Y.345 | CH₃ | H | (CH₂)₂CH₃ | F | 4-F | 2-Cl | H |
| Y.346 | CH₃ | H | CH(CH₃)₂ | F | 4-F | 2-Cl | H |
| Y.347 | CH₃ | H | F | F | 4-F | 2-Cl | H |
| Y.348 | (CH₂)₂ | | H | H | 4-F | 2-Cl | H |
| Y.349 | (CH₂)₂ | | CH₃ | H | 4-F | 2-Cl | H |
| Y.350 | (CH₂)₂ | | (CH₂)₂CH₃ | H | 4-F | 2-Cl | H |
| Y.351 | (CH₂)₂ | | CH(CH₃)₂ | H | 4-F | 2-Cl | H |
| Y.352 | H | H | H | H | 4-p-Cl-phenyl | 2-Cl | H |
| Y.353 | CH₃ | H | H | H | 4-p-Cl-phenyl | 2-Cl | H |
| Y.354 | CH₂CH₃ | H | H | H | 4-p-Cl-phenyl | 2-Cl | H |
| Y.355 | (CH₂)₂CH₃ | H | H | H | 4-p-Cl-phenyl | 2-Cl | H |
| Y.356 | CH(CH₃)₂ | H | H | H | 4-p-Cl-phenyl | 2-Cl | H |
| Y.347 | F | H | H | H | 4-p-Cl-phenyl | 2-Cl | H |
| Y.358 | H | H | CH₃ | H | 4-p-Cl-phenyl | 2-Cl | H |
| Y.359 | H | H | CH₂CH₃ | H | 4-p-Cl-phenyl | 2-Cl | H |
| Y.360 | H | H | (CH₂)₂CH₃ | H | 4-p-Cl-phenyl | 2-Cl | H |
| Y.361 | H | H | CH(CH₃)₂ | H | 4-p-Cl-phenyl | 2-Cl | H |
| Y.362 | H | H | F | H | 4-p-Cl-phenyl | 2-Cl | H |
| Y.363 | CH₃ | CH₃ | H | H | 4-p-Cl-phenyl | 2-Cl | H |
| Y.364 | CH₂CH₃ | CH₃ | H | H | 4-p-Cl-phenyl | 2-Cl | H |
| Y.365 | (CH₂)₂CH₃ | CH₃ | H | H | 4-p-Cl-phenyl | 2-Cl | H |
| Y.366 | CH(CH₃)₂ | CH₃ | H | H | 4-p-Cl-phenyl | 2-Cl | H |
| Y.367 | F | CH₃ | H | H | 4-p-Cl-phenyl | 2-Cl | H |
| Y.368 | CH₃ | H | CH₃ | H | 4-p-Cl-phenyl | 2-Cl | H |
| Y.369 | CH₂CH₃ | H | CH₃ | H | 4-p-Cl-phenyl | 2-Cl | H |
| Y.370 | (CH₂)₂CH₃ | H | CH₃ | H | 4-p-Cl-phenyl | 2-Cl | H |
| Y.371 | CH(CH₃)₂ | H | CH₃ | H | 4-p-Cl-phenyl | 2-Cl | H |
| Y.372 | CH₃ | H | F | H | 4-p-Cl-phenyl | 2-Cl | H |
| Y.373 | CH₂CH₃ | H | F | H | 4-p-Cl-phenyl | 2-Cl | H |
| Y.374 | H | H | CH₃ | CH₃ | 4-p-Cl-phenyl | 2-Cl | H |
| Y.375 | H | H | CH₂CH₃ | CH₃ | 4-p-Cl-phenyl | 2-Cl | H |
| Y.376 | H | H | CH(CH₃)₂ | CH₃ | 4-p-Cl-phenyl | 2-Cl | H |
| Y.377 | H | H | CH₃ | F | 4-p-Cl-phenyl | 2-Cl | H |
| Y.378 | H | H | CH₂CH₃ | F | 4-p-Cl-phenyl | 2-Cl | H |
| Y.379 | H | H | (CH₂)₂CH₃ | F | 4-p-Cl-phenyl | 2-Cl | H |
| Y.380 | H | H | CH(CH₃)₂ | F | 4-p-Cl-phenyl | 2-Cl | H |
| Y.381 | H | H | F | F | 4-p-Cl-phenyl | 2-Cl | H |
| Y.382 | CH₃ | H | CH₃ | F | 4-p-Cl-phenyl | 2-Cl | H |
| Y.383 | CH₃ | H | CH₂CH₃ | F | 4-p-Cl-phenyl | 2-Cl | H |
| Y.384 | CH₃ | H | (CH₂)₂CH₃ | F | 4-p-Cl-phenyl | 2-Cl | H |
| Y.385 | CH₃ | H | CH(CH₃)₂ | F | 4-p-Cl-phenyl | 2-Cl | H |
| Y.386 | CH₃ | H | F | F | 4-p-Cl-phenyl | 2-Cl | H |
| Y.387 | (CH₂)₂ | | H | H | 4-p-Cl-phenyl | 2-Cl | H |
| Y.388 | (CH₂)₂ | | CH₃ | H | 4-p-Cl-phenyl | 2-Cl | H |
| Y.389 | (CH₂)₂ | | (CH₂)₂CH₃ | H | 4-p-Cl-phenyl | 2-Cl | H |
| Y.390 | (CH₂)₂ | | CH(CH₃)₂ | H | 4-p-Cl-phenyl | 2-Cl | H |
| Y.391 | H | H | H | H | 2-Cl | 4-Cl | 6-Cl |
| Y.392 | CH₃ | H | H | H | 2-Cl | 4-Cl | 6-Cl |
| Y.393 | CH₂CH₃ | H | H | H | 2-Cl | 4-Cl | 6-Cl |
| Y.394 | (CH₂)₂CH₃ | H | H | H | 2-Cl | 4-Cl | 6-Cl |
| Y.395 | CH(CH₃)₂ | H | H | H | 2-Cl | 4-Cl | 6-Cl |
| Y.396 | F | H | H | H | 2-Cl | 4-Cl | 6-Cl |
| Y.397 | H | H | CH₃ | H | 2-Cl | 4-Cl | 6-Cl |
| Y.398 | H | H | CH₂CH₃ | H | 2-Cl | 4-Cl | 6-Cl |
| Y.399 | CH₃ | CH₃ | H | H | 2-Cl | 4-Cl | 6-Cl |
| Y.400 | CH₂CH₃ | CH₃ | H | H | 2-Cl | 4-Cl | 6-Cl |
| Y.401 | F | CH₃ | H | H | 2-Cl | 4-Cl | 6-Cl |
| Y.402 | CH₃ | H | CH₃ | H | 2-Cl | 4-Cl | 6-Cl |
| Y.403 | CH₂CH₃ | H | CH₃ | H | 2-Cl | 4-Cl | 6-Cl |
| Y.404 | CH₃ | H | F | H | 2-Cl | 4-Cl | 6-Cl |
| Y.405 | CH₂CH₃ | H | F | H | 2-Cl | 4-Cl | 6-Cl |
| Y.406 | H | H | CH₃ | CH₃ | 2-Cl | 4-Cl | 6-Cl |
| Y.407 | H | H | CH₂CH₃ | CH₃ | 2-Cl | 4-Cl | 6-Cl |
| Y.408 | H | H | CH₃ | F | 2-Cl | 4-Cl | 6-Cl |
| Y.409 | H | H | CH₂CH₃ | F | 2-Cl | 4-Cl | 6-Cl |
| Y.410 | H | H | F | F | 2-Cl | 4-Cl | 6-Cl |
| Y.411 | CH₃ | H | CH₃ | F | 2-Cl | 4-Cl | 6-Cl |
| Y.412 | CH₃ | H | CH₂CH₃ | F | 2-Cl | 4-Cl | 6-Cl |
| Y.413 | CH₃ | H | F | F | 2-Cl | 4-Cl | 6-Cl |
| Y.414 | (CH₂)₂ | | H | H | 2-Cl | 4-Cl | 6-Cl |
| Y.415 | H | H | H | H | 2-F | 4-F | 6-F |
| Y.416 | CH₃ | H | H | H | 2-F | 4-F | 6-F |
| Y.417 | CH₂CH₃ | H | H | H | 2-F | 4-F | 6-F |
| Y.418 | (CH₂)₂CH₃ | H | H | H | 2-F | 4-F | 6-F |
| Y.419 | CH(CH₃)₂ | H | H | H | 2-F | 4-F | 6-F |
| Y.420 | F | H | H | H | 2-F | 4-F | 6-F |
| Y.421 | H | H | CH₃ | H | 2-F | 4-F | 6-F |
| Y.422 | H | H | CH₂CH₃ | H | 2-F | 4-F | 6-F |
| Y.423 | CH₃ | CH₃ | H | H | 2-F | 4-F | 6-F |
| Y.424 | CH₂CH₃ | CH₃ | H | H | 2-F | 4-F | 6-F |
| Y.425 | F | CH₃ | H | H | 2-F | 4-F | 6-F |
| Y.426 | CH₃ | H | CH₃ | H | 2-F | 4-F | 6-F |
| Y.427 | CH₂CH₃ | H | CH₃ | H | 2-F | 4-F | 6-F |
| Y.428 | CH₃ | H | F | H | 2-F | 4-F | 6-F |
| Y.429 | CH₂CH₃ | H | F | H | 2-F | 4-F | 6-F |
| Y.430 | H | H | CH₃ | CH₃ | 2-F | 4-F | 6-F |
| Y.431 | H | H | CH₂CH₃ | CH₃ | 2-F | 4-F | 6-F |
| Y.432 | H | H | CH₃ | F | 2-F | 4-F | 6-F |
| Y.433 | H | H | CH₂CH₃ | F | 2-F | 4-F | 6-F |
| Y.434 | H | H | F | F | 2-F | 4-F | 6-F |
| Y.435 | CH₃ | H | CH₃ | F | 2-F | 4-F | 6-F |
| Y.436 | CH₃ | H | CH₂CH₃ | F | 2-F | 4-F | 6-F |
| Y.437 | CH₃ | H | F | F | 2-F | 4-F | 6-F |
| Y.438 | (CH₂)₂ | | H | H | 2-F | 4-F | 6-F |
| Y.439 | CF₃ | H | H | H | 2-Cl | 4-Cl | H |
| Y.440 | CF₂H | H | H | H | 2-Cl | 4-Cl | H |
| Y.441 | CH₃ | H | H | H | 2-Cl | H | 6-F |
| Y.442 | CH₃ | H | H | H | 2-Cl | H | 6-Cl |
| Y.443 | CH₃ | H | H | H | 2-Cl | H | 6-CH₃ |
| Y.444 | CH₂F | H | H | H | 2-Cl | 4-Cl | H |
| Y.445 | CF₃ | H | H | H | 2-Cl | 4-Cl | 6-Cl |
| Y.446 | H | H | H | H | 4-OH | H | H |
| Y.447 | H | H | H | H | 4-O-(4′-CF₃- | H | H |
| | | | | | phenyl) | | |
| Y.448 | H | H | H | H | 4-O-(3-Cl,5- | H | H |
| | | | | | CF₃-2- | | |
| | | | | | pyridinyl) | | |
| Y.449 | CH₃ | H | H | H | 4-O-(3-Cl,5- | 2-Cl | 6-Cl |
| | | | | | CF₃-2- | | |
| | | | | | pyridinyl) | | |
| Y.450 | CH₃ | H | H | H | 4-O-(4′-CF₃- | 2-Cl | 6-Cl |
| | | | | | phenyl) | | |
| Y.451 | CH₃ | H | H | H | 2-Cl | 4-Br | H |
| Y.452 | CH₃ | H | F | H | 2-Cl | 4-Br | H |
| Y.453 | CH₃ | H | CH₃ | H | 2-Cl | 4-Br | H |
| Y.454 | CF₂H | H | H | H | 2-Cl | 4-Cl | 6-Cl |
| Y.455 | CH₂F | H | H | H | 2-Cl | 4-Cl | 6-Cl |
| Y.456 | CH₃ | H | H | H | 2-Cl | 4-Br | 6-Cl |
| Y.457 | CH₃ | H | F | H | 2-Cl | 4-Br | 6-Cl |
| Y.458 | CH₃ | H | CH₃ | H | 2-Cl | 4-Br | 6-Cl |
| Y.459 | CF₃ | H | H | H | 2-Br | 4-Br | 6-Cl |
| Y.460 | CF₃ | H | H | H | 2-Br | 4-Br | 6-Cl |
| Y.461 | CH₃ | H | H | H | 4-(2′-Cl- | 2-Cl | H |
| | | | | | phenyl) | | |
| Y.462 | CH₃ | H | H | H | 4-(4′-Cl- | 2-Cl | H |
| | | | | | phenyl) | | |
| Y.463 | CH₃ | H | H | H | 4-(4′-CF₃- | 2-Cl | H |
| | | | | | phenyl) | | |
| Y.464 | CH₃ | H | H | H | 4-(4′-OCH₃- | 2-Cl | H |
| | | | | | phenyl) | | |
| Y.465 | CH₃ | H | H | H | 4-(3′,4′-Cl₂- | 2-Cl | H |
| | | | | | phenyl) | | |
| Y.466 | CH₃ | H | H | H | 4-(2′-Cl- | 2-Cl | 6-Cl |
| | | | | | phenyl) | | |
| Y.467 | CH₃ | H | H | H | 4-(4′-CF₃- | 2-Cl | 6-Cl |
| | | | | | phenyl) | | |
| Y.468 | CH₃ | H | H | H | 4-(4′-OCH₃- | 2-Cl | 6-Cl |
| | | | | | phenyl) | | |
| Y.469 | CH₃ | H | H | H | 4-(3′,4′-Cl₂- | 2-Cl | 6-Cl |
| | | | | | phenyl) | | |
| Y.470 | CH₃ | H | H | H | 4-C≡CSi(CH₃)₃ | 2-Cl | H |
| Y.471 | CH₃ | H | H | H | 4-C≡CH | 2-Cl | H |
| Y.472 | CH₃ | H | H | H | 4-C≡CC(CH₃)₃ | 2-Cl | H |
| Y.473 | CH₃ | H | H | H | 4- | 2-Cl | H |
| | | | | | C≡CCH(CH₂)₂ | | |
| Y.474 | CH₃ | H | H | H | 4-C≡C-(4-Cl- | 2-Cl | H |
| | | | | | phenyl) | | |
| Y.475 | CH₃ | H | H | H | 4-C≡CC(CH₃)₃ | 2-Cl | 6-Cl |
| Y.476 | CH₃ | H | H | H | 4- | 2-Cl | 6-Cl |
| | | | | | C≡CCH(CH₂)₂ | | |
| Y.477 | CH₃ | H | H | H | 4-C≡C-(4-Cl- | 2-Cl | 6-Cl |
| | | | | | phenyl) | | |
| Y.478 | CH₃ | H | H | H | 4-CHO | 2-Cl | H |
| Y.479 | CH₃ | H | H | H | 4-CH═NOCH₃ | 2-Cl | H |
| Y.480 | CH₃ | H | H | H | 4-COCH₃ | 2-Cl | H |
| Y.481 | CH₃ | H | H | H | 4- | 2-Cl | H |
| | | | | | C(CH₃)═NOCH₃ | | |
| Y.482 | CH₃ | H | H | H | 4-CH₃ | 2-CH₃ | 6-CH₃ |

Table 1 provides 482 compounds of formula (IA), wherein A is

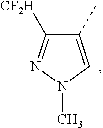

wherein the broken lines indicate the point of attachment of the group A to the amide group, and R₁, R₂, R₃, R₄, R₈ₐ, R_{8b} and R_{8c} are as defined in Table Y. For example, compound 1.001 has the following structure:

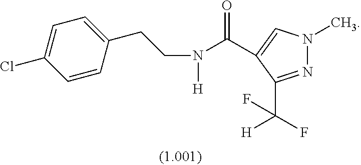

Table 2 provides 482 compounds of formula (IA) wherein A is

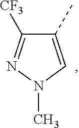

wherein the broken lines indicate the point of attachment of the group A to the amide group, and R₁, R₂, R₃, R₄, R₈ₐ, R_{8b} and R_{8c} are as defined in Table Y.

Table 3 provides 482 compounds of formula (IA) wherein A is

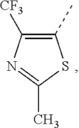

wherein the broken lines indicate the point of attachment of the group A to the amide group, and R₁, R₂, R₃, R₄, R₈ₐ, R_{8b} and R_{8c} are as defined in Table Y.

Table 4 provides 482 compounds of formula (IA) wherein A is

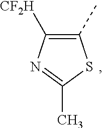

wherein the broken lines indicate the point of attachment of the group A to the amide group, and R₁, R₂, R₃, R₄, R₈ₐ, R_{8b} and R_{8c} are as defined in Table Y.

Table 5 provides 482 compounds of formula (IA) wherein A is

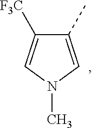

wherein the broken lines indicate the point of attachment of the group A to the amide group, and R₁, R₂, R₃, R₄, R₈ₐ, R_{8b} and R_{8c} are as defined in Table Y.

Table 6 provides 482 compounds of formula (IA) wherein A is

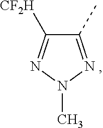

wherein the broken lines indicate the point of attachment of the group A to the amide group, and R₁, R₂, R₃, R₄, R₈ₐ, R_{8b} and R_{8c} are as defined in Table Y.

Table 7 provides 482 compounds of formula (IA) wherein A is

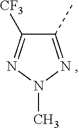

wherein the broken lines indicate the point of attachment of the group A to the amide group, and R₁, R₂, R₃, R₄, R₈ₐ, R_{8b} and R_{8c} are as defined in Table Y.

### Tables 8 to 12: Compounds of Formula IB

The invention is further illustrated by the preferred individual compounds of formula (IB) listed below in Tables 8 to 12. Characterising data is given in Table 14.

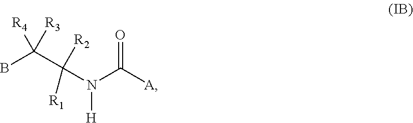

Each of Tables 8 to 12, which follow the Table W below, comprises 288 compounds of the formula (IB) in which B, R₁, R₂, R₃ and R₄ have the values given in Table W and A has the value given in the relevant Table 8 to 12. Thus Table 8 corresponds to Table W when W is 8 and A has the value given under the Table 8 heading, Table 9 corresponds to Table W when W is 9 and A has the value given under the Table 9 heading, and so on for Tables 10 to 12.

TABLE W
In Table W the group B stands for the group B₁, B₂, B₃ or B₄:
(B₁)

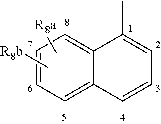

(B₂)

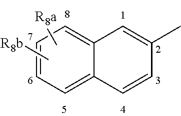

(B₃)

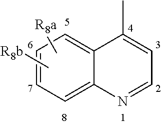

(B₄)

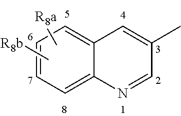

| Compound | | | | | | | |
|---|---|---|---|---|---|---|---|
| No. | B | R₁ | R₂ | R₃ | R₄ | R₈ₐ | R_{8b} |
| W.001 | B₁ | H | H | H | H | 2-Cl | H |
| W.002 | B₁ | CH₃ | H | H | H | 2-Cl | H |
| W.003 | B₁ | CH₂CH₃ | H | H | H | 2-Cl | H |
| W.004 | B₁ | CH₃ | CH₃ | H | H | 2-Cl | H |
| W.005 | B₁ | CH₃ | CH₂CH₃ | H | H | 2-Cl | H |
| W.006 | B₁ | CH₃ | H | F | H | 2-Cl | H |
| W.007 | B₁ | CH₃ | CH₃ | F | H | 2-Cl | H |
| W.008 | B₁ | CH₃ | H | F | F | 2-Cl | H |
| W.009 | B₁ | CH₃ | H | CH₃ | CH₃ | 2-Cl | H |
| W.010 | B₁ | H | H | H | H | 4-Cl | H |
| W.011 | B₁ | CH₃ | H | H | H | 4-Cl | H |
| W.012 | B₁ | CH₂CH₃ | H | H | H | 4-Cl | H |
| W.013 | B₁ | CH₃ | CH₃ | H | H | 4-Cl | H |
| W.014 | B₁ | CH₃ | CH₂CH₃ | H | H | 4-Cl | H |
| W.015 | B₁ | CH₃ | H | F | H | 4-Cl | H |
| W.016 | B₁ | CH₃ | CH₃ | F | H | 4-Cl | H |
| W.017 | B₁ | CH₃ | H | F | F | 4-Cl | H |
| W.018 | B₁ | CH₃ | H | CH₃ | CH₃ | 4-Cl | H |
| W.019 | B₁ | H | H | H | H | 5-Cl | H |
| W.020 | B₁ | CH₃ | H | H | H | 5-Cl | H |
| W.021 | B₁ | CH₂CH₃ | H | H | H | 5-Cl | H |
| W.022 | B₁ | CH₃ | CH₃ | H | H | 5-Cl | H |
| W.023 | B₁ | CH₃ | CH₂CH₃ | H | H | 5-Cl | H |
| W.024 | B₁ | CH₃ | H | F | H | 5-Cl | H |
| W.025 | B₁ | CH₃ | CH₃ | F | H | 5-Cl | H |
| W.026 | B₁ | CH₃ | H | F | F | 5-Cl | H |
| W.027 | B₁ | CH₃ | H | CH₃ | CH₃ | 5-Cl | H |
| W.028 | B₁ | H | H | H | H | 6-Cl | H |
| W.029 | B₁ | CH₃ | H | H | H | 6-Cl | H |
| W.030 | B₁ | CH₂CH₃ | H | H | H | 6-Cl | H |
| W.031 | B₁ | CH₃ | CH₃ | H | H | 6-Cl | H |
| W.032 | B₁ | CH₃ | CH₂CH₃ | H | H | 6-Cl | H |
| W.033 | B₁ | CH₃ | H | F | H | 6-Cl | H |
| W.034 | B₁ | CH₃ | CH₃ | F | H | 6-Cl | H |
| W.035 | B₁ | CH₃ | H | F | F | 6-Cl | H |
| W.036 | B₁ | CH₃ | H | CH₃ | CH₃ | 6-Cl | H |
| W.037 | B₁ | H | H | H | H | 8-Cl | H |
| W.038 | B₁ | CH₃ | H | H | H | 8-Cl | H |
| W.039 | B₁ | CH₂CH₃ | H | H | H | 8-Cl | H |
| W.040 | B₁ | CH₃ | CH₃ | H | H | 8-Cl | H |
| W.041 | B₁ | CH₃ | CH₂CH₃ | H | H | 8-Cl | H |
| W.042 | B₁ | CH₃ | H | F | H | 8-Cl | H |
| W.043 | B₁ | CH₃ | CH₃ | F | H | 8-Cl | H |
| W.044 | B₁ | CH₃ | H | F | F | 8-Cl | H |
| W.045 | B₁ | CH₃ | H | CH₃ | CH₃ | 8-Cl | H |
| W.046 | B₁ | H | H | H | H | 4-p-Cl-phenyl | H |
| W.047 | B₁ | CH₃ | H | H | H | 4-p-Cl-phenyl | H |
| W.048 | B₁ | CH₂CH₃ | H | H | H | 4-p-Cl-phenyl | H |
| W.049 | B₁ | CH₃ | CH₃ | H | H | 4-p-Cl-phenyl | H |
| W.050 | B₁ | CH₃ | CH₂CH₃ | H | H | 4-p-Cl-phenyl | H |
| W.051 | B₁ | CH₃ | H | F | H | 4-p-Cl-phenyl | H |
| W.052 | B₁ | CH₃ | CH₃ | F | H | 4-p-Cl-phenyl | H |
| W.053 | B₁ | CH₃ | H | F | F | 4-p-Cl-phenyl | H |
| W.054 | B₁ | CH₃ | H | CH₃ | CH₃ | 4-p-Cl-phenyl | H |
| W.055 | B₁ | H | H | H | H | 8-p-Cl-phenyl | H |
| W.056 | B₁ | CH₃ | H | H | H | 8-p-Cl-phenyl | H |
| W.057 | B₁ | CH₂CH₃ | H | H | H | 8-p-Cl-phenyl | H |
| W.058 | B₁ | CH₃ | CH₃ | H | H | 8-p-Cl-phenyl | H |
| W.059 | B₁ | CH₃ | CH₂CH₃ | H | H | 8-p-Cl-phenyl | H |
| W.060 | B₁ | CH₃ | H | F | H | 8-p-Cl-phenyl | H |
| W.061 | B₁ | CH₃ | CH₃ | F | H | 8-p-Cl-phenyl | H |
| W.062 | B₁ | CH₃ | H | F | F | 8-p-Cl-phenyl | H |
| W.063 | B₁ | CH₃ | H | CH₃ | CH₃ | 8-p-Cl-phenyl | H |
| W.064 | B₁ | H | H | H | H | 2-Cl | 4-Cl |
| W.065 | B₁ | CH₃ | H | H | H | 2-Cl | 4-Cl |
| W.066 | B₁ | CH₂CH₃ | H | H | H | 2-Cl | 4-Cl |
| W.067 | B₁ | CH₃ | CH₃ | H | H | 2-Cl | 4-Cl |
| W.068 | B₁ | CH₃ | CH₂CH₃ | H | H | 2-Cl | 4-Cl |
| W.069 | B₁ | CH₃ | H | F | H | 2-Cl | 4-Cl |
| W.070 | B₁ | CH₃ | CH₃ | F | H | 2-Cl | 4-Cl |
| W.071 | B₁ | CH₃ | H | F | F | 2-Cl | 4-Cl |
| W.072 | B₁ | CH₃ | H | CH₃ | CH₃ | 2-Cl | 4-Cl |
| W.073 | B₁ | H | H | H | H | 4-p-Cl-phenyl | 2-Cl |
| W.074 | B₁ | CH₃ | H | H | H | 4-p-Cl-phenyl | 2-Cl |
| W.075 | B₁ | CH₂CH₃ | H | H | H | 4-p-Cl-phenyl | 2-Cl |
| W.076 | B₁ | CH₃ | CH₃ | H | H | 4-p-Cl-phenyl | 2-Cl |
| W.077 | B₁ | CH₃ | CH₂CH₃ | H | H | 4-p-Cl-phenyl | 2-Cl |
| W.078 | B₁ | CH₃ | H | F | H | 4-p-Cl-phenyl | 2-Cl |
| W.079 | B₁ | CH₃ | CH₃ | F | H | 4-p-Cl-phenyl | 2-Cl |
| W.080 | B₁ | CH₃ | H | F | F | 4-p-Cl-phenyl | 2-Cl |
| W.081 | B₁ | CH₃ | H | CH₃ | CH₃ | 4-p-Cl-phenyl | 2-Cl |
| W.082 | B₂ | H | H | H | H | 6-Cl | H |
| W.083 | B₂ | CH₃ | H | H | H | 6-Cl | H |
| W.084 | B₂ | CH₂CH₃ | H | H | H | 6-Cl | H |
| W.085 | B₂ | CH₃ | CH₃ | H | H | 6-Cl | H |
| W.086 | B₂ | CH₃ | CH₂CH₃ | H | H | 6-Cl | H |
| W.087 | B₂ | CH₃ | H | F | H | 6-Cl | H |
| W.088 | B₂ | CH₃ | CH₃ | F | H | 6-Cl | H |
| W.089 | B₂ | CH₃ | H | F | F | 6-Cl | H |
| W.090 | B₂ | CH₃ | H | CH₃ | CH₃ | 6-Cl | H |
| W.091 | B₂ | H | H | H | H | 6-CF₃ | H |
| W.092 | B₂ | CH₃ | H | H | H | 6-CF₃ | H |
| W.093 | B₂ | CH₂CH₃ | H | H | H | 6-CF₃ | H |
| W.094 | B₂ | CH₃ | CH₃ | H | H | 6-CF₃ | H |
| W.095 | B₂ | CH₃ | CH₂CH₃ | H | H | 6-CF₃ | H |
| W.096 | B₂ | CH₃ | H | F | H | 6-CF₃ | H |
| W.097 | B₂ | CH₃ | CH₃ | F | H | 6-CF₃ | H |
| W.098 | B₂ | CH₃ | H | F | F | 6-CF₃ | H |
| W.099 | B₂ | CH₃ | H | CH₃ | CH₃ | 6-CF₃ | H |
| W.100 | B₂ | H | H | H | H | 6-OCF₃ | H |
| W.101 | B₂ | CH₃ | H | H | H | 6-OCF₃ | H |
| W.102 | B₂ | CH₂CH₃ | H | H | H | 6-OCF₃ | H |
| W.103 | B₂ | CH₃ | CH₃ | H | H | 6-OCF₃ | H |
| W.104 | B₂ | CH₃ | CH₂CH₃ | H | H | 6-OCF₃ | H |
| W.105 | B₂ | CH₃ | H | F | H | 6-OCF₃ | H |
| W.106 | B₂ | CH₃ | CH₃ | F | H | 6-OCF₃ | H |
| W.107 | B₂ | CH₃ | H | F | F | 6-OCF₃ | H |
| W.108 | B₂ | CH₃ | H | CH₃ | CH₃ | 6-OCF₃ | H |
| W.109 | B₂ | H | H | H | H | 6-p-Cl-phenyl | H |
| W.110 | B₂ | CH₃ | H | H | H | 6-p-Cl-phenyl | H |
| W.111 | B₂ | CH₂CH₃ | H | H | H | 6-p-Cl-phenyl | H |
| W.112 | B₂ | CH₃ | CH₃ | H | H | 6-p-Cl-phenyl | H |
| W.113 | B₂ | CH₃ | CH₂CH₃ | H | H | 6-p-Cl-phenyl | H |
| W.114 | B₂ | CH₃ | H | F | H | 6-p-Cl-phenyl | H |
| W.115 | B₂ | CH₃ | CH₃ | F | H | 6-p-Cl-phenyl | H |
| W.116 | B₂ | CH₃ | H | F | F | 6-p-Cl-phenyl | H |
| W.117 | B₂ | CH₃ | H | CH₃ | CH₃ | 6-p-Cl-phenyl | H |
| W.118 | B₃ | H | H | H | H | 2-Cl | H |
| W.119 | B₃ | CH₃ | H | H | H | 2-Cl | H |
| W.120 | B₃ | CH₂CH₃ | H | H | H | 2-Cl | H |
| W.121 | B₃ | CH₃ | CH₃ | H | H | 2-Cl | H |
| W.122 | B₃ | CH₃ | CH₂CH₃ | H | H | 2-Cl | H |
| W.123 | B₃ | CH₃ | H | F | H | 2-Cl | H |
| W.124 | B₃ | CH₃ | CH₃ | F | H | 2-Cl | H |
| W.125 | B₃ | CH₃ | H | F | F | 2-Cl | H |
| W.126 | B₃ | CH₃ | H | CH₃ | CH₃ | 2-Cl | H |
| W.127 | B₃ | H | H | H | H | 3-Cl | H |
| W.128 | B₃ | CH₃ | H | H | H | 3-Cl | H |
| W.129 | B₃ | CH₂CH₃ | H | H | H | 3-Cl | H |
| W.130 | B₃ | CH₃ | CH₃ | H | H | 3-Cl | H |
| W.131 | B₃ | CH₃ | CH₂CH₃ | H | H | 3-Cl | H |
| W.132 | B₃ | CH₃ | H | F | H | 3-Cl | H |
| W.133 | B₃ | CH₃ | CH₃ | F | H | 3-Cl | H |
| W.134 | B₃ | CH₃ | H | F | F | 3-Cl | H |
| W.135 | B₃ | CH₃ | H | CH₃ | CH₃ | 3-Cl | H |
| W.136 | B₃ | H | H | H | H | 5-Cl | H |
| W.137 | B₃ | CH₃ | H | H | H | 5-Cl | H |
| W.138 | B₃ | CH₂CH₃ | H | H | H | 5-Cl | H |
| W.139 | B₃ | CH₃ | CH₃ | H | H | 5-Cl | H |
| W.140 | B₃ | CH₃ | CH₂CH₃ | H | H | 5-Cl | H |
| W.141 | B₃ | CH₃ | H | F | H | 5-Cl | H |
| W.142 | B₃ | CH₃ | CH₃ | F | H | 5-Cl | H |
| W.143 | B₃ | CH₃ | H | F | F | 5-Cl | H |
| W.144 | B₃ | CH₃ | H | CH₃ | CH₃ | 5-Cl | H |
| W.145 | B₃ | H | H | H | H | 6-Cl | H |
| W.146 | B₃ | CH₃ | H | H | H | 6-Cl | H |
| W.147 | B₃ | CH₂CH₃ | H | H | H | 6-Cl | H |
| W.148 | B₃ | CH₃ | CH₃ | H | H | 6-Cl | H |
| W.149 | B₃ | CH₃ | CH₂CH₃ | H | H | 6-Cl | H |
| W.150 | B₃ | CH₃ | H | F | H | 6-Cl | H |
| W.151 | B₃ | CH₃ | CH₃ | F | H | 6-Cl | H |
| W.152 | B₃ | CH₃ | H | F | F | 6-Cl | H |
| W.153 | B₃ | CH₃ | H | CH₃ | CH₃ | 6-Cl | H |
| W.154 | B₃ | H | H | H | H | 8-Cl | H |
| W.155 | B₃ | CH₃ | H | H | H | 8-Cl | H |
| W.156 | B₃ | CH₂CH₃ | H | H | H | 8-Cl | H |
| W.157 | B₃ | CH₃ | CH₃ | H | H | 8-Cl | H |
| W.158 | B₃ | CH₃ | CH₂CH₃ | H | H | 8-Cl | H |
| W.159 | B₃ | CH₃ | H | F | H | 8-Cl | H |
| W.160 | B₃ | CH₃ | CH₃ | F | H | 8-Cl | H |
| W.161 | B₃ | CH₃ | H | F | F | 8-Cl | H |
| W.162 | B₃ | CH₃ | H | CH₃ | CH₃ | 8-Cl | H |
| W.163 | B₃ | H | H | H | H | 2-Cl | 5-Cl |
| W.164 | B₃ | CH₃ | H | H | H | 2-Cl | 5-Cl |
| W.165 | B₃ | CH₂CH₃ | H | H | H | 2-Cl | 5-Cl |
| W.166 | B₃ | CH₃ | CH₃ | H | H | 2-Cl | 5-Cl |
| W.167 | B₃ | CH₃ | CH₂CH₃ | H | H | 2-Cl | 5-Cl |
| W.168 | B₃ | CH₃ | H | F | H | 2-Cl | 5-Cl |
| W.169 | B₃ | CH₃ | CH₃ | F | H | 2-Cl | 5-Cl |
| W.170 | B₃ | CH₃ | H | F | F | 2-Cl | 5-Cl |
| W.171 | B₃ | CH₃ | H | CH₃ | CH₃ | 2-Cl | 5-Cl |
| W.172 | B₃ | H | H | H | H | 2-Cl | 6-Cl |
| W.173 | B₃ | CH₃ | H | H | H | 2-Cl | 6-Cl |
| W.174 | B₃ | CH₂CH₃ | H | H | H | 2-Cl | 6-Cl |
| W.175 | B₃ | CH₃ | CH₃ | H | H | 2-Cl | 6-Cl |
| W.176 | B₃ | CH₃ | CH₂CH₃ | H | H | 2-Cl | 6-Cl |
| W.177 | B₃ | CH₃ | H | F | H | 2-Cl | 6-Cl |
| W.178 | B₃ | CH₃ | CH₃ | F | H | 2-Cl | 6-Cl |
| W.179 | B₃ | CH₃ | H | F | F | 2-Cl | 6-Cl |
| W.180 | B₃ | CH₃ | H | CH₃ | CH₃ | 2-Cl | 6-Cl |
| W.181 | B₃ | H | H | H | H | 2-Cl | 8-Cl |
| W.182 | B₃ | CH₃ | H | H | H | 2-Cl | 8-Cl |
| W.183 | B₃ | CH₂CH₃ | H | H | H | 2-Cl | 8-Cl |
| W.184 | B₃ | CH₃ | CH₃ | H | H | 2-Cl | 8-Cl |
| W.185 | B₃ | CH₃ | CH₂CH₃ | H | H | 2-Cl | 8-Cl |
| W.186 | B₃ | CH₃ | H | F | H | 2-Cl | 8-Cl |
| W.187 | B₃ | CH₃ | CH₃ | F | H | 2-Cl | 8-Cl |
| W.188 | B₃ | CH₃ | H | F | F | 2-Cl | 8-Cl |
| W.189 | B₃ | CH₃ | H | CH₃ | CH₃ | 2-Cl | 8-Cl |
| W.190 | B₃ | H | H | H | H | 6-p-Cl-phenyl | 2-Cl |
| W.191 | B₃ | CH₃ | H | H | H | 6-p-Cl-phenyl | 2-Cl |
| W.192 | B₃ | CH₂CH₃ | H | H | H | 6-p-Cl-phenyl | 2-Cl |
| W.193 | B₃ | CH₃ | CH₃ | H | H | 6-p-Cl-phenyl | 2-Cl |
| W.194 | B₃ | CH₃ | CH₂CH₃ | H | H | 6-p-Cl-phenyl | 2-Cl |
| W.195 | B₃ | CH₃ | H | F | H | 6-p-Cl-phenyl | 2-Cl |
| W.196 | B₃ | CH₃ | CH₃ | F | H | 6-p-Cl-phenyl | 2-Cl |
| W.197 | B₃ | CH₃ | H | F | F | 6-p-Cl-phenyl | 2-Cl |
| W.198 | B₃ | CH₃ | H | CH₃ | CH₃ | 6-p-Cl-phenyl | 2-Cl |
| W.199 | B₄ | H | H | H | H | 2-Cl | H |
| W.200 | B₄ | CH₃ | H | H | H | 2-Cl | H |
| W.201 | B₄ | CH₂CH₃ | H | H | H | 2-Cl | H |
| W.202 | B₄ | CH₃ | CH₃ | H | H | 2-Cl | H |
| W.203 | B₄ | CH₃ | CH₂CH₃ | H | H | 2-Cl | H |
| W.204 | B₄ | CH₃ | H | F | H | 2-Cl | H |
| W.205 | B₄ | CH₃ | CH₃ | F | H | 2-Cl | H |
| W.206 | B₄ | CH₃ | H | F | F | 2-Cl | H |
| W.207 | B₄ | CH₃ | H | CH₃ | CH₃ | 2-Cl | H |
| W.208 | B₄ | H | H | H | H | 4-Cl | H |
| W.209 | B₄ | CH₃ | H | H | H | 4-Cl | H |
| W.210 | B₄ | CH₂CH₃ | H | H | H | 4-Cl | H |
| W.211 | B₄ | CH₃ | CH₃ | H | H | 4-Cl | H |
| W.212 | B₄ | CH₃ | CH₂CH₃ | H | H | 4-Cl | H |
| W.213 | B₄ | CH₃ | H | F | H | 4-Cl | H |
| W.214 | B₄ | CH₃ | CH₃ | F | H | 4-Cl | H |
| W.215 | B₄ | CH₃ | H | F | F | 4-Cl | H |
| W.216 | B₄ | CH₃ | H | CH₃ | CH₃ | 4-Cl | H |
| W.217 | B₄ | H | H | H | H | 5-Cl | H |
| W.218 | B₄ | CH₃ | H | H | H | 5-Cl | H |
| W.219 | B₄ | CH₂CH₃ | H | H | H | 5-Cl | H |
| W.220 | B₄ | CH₃ | CH₃ | H | H | 5-Cl | H |
| W.221 | B₄ | CH₃ | CH₂CH₃ | H | H | 5-Cl | H |
| W.222 | B₄ | CH₃ | H | F | H | 5-Cl | H |
| W.223 | B₄ | CH₃ | CH₃ | F | H | 5-Cl | H |
| W.224 | B₄ | CH₃ | H | F | F | 5-Cl | H |
| W.225 | B₄ | CH₃ | H | CH₃ | CH₃ | 5-Cl | H |
| W.226 | B₄ | H | H | H | H | 7-Cl | H |
| W.227 | B₄ | CH₃ | H | H | H | 7-Cl | H |
| W.228 | B₄ | CH₂CH₃ | H | H | H | 7-Cl | H |
| W.229 | B₄ | CH₃ | CH₃ | H | H | 7-Cl | H |
| W.230 | B₄ | CH₃ | CH₂CH₃ | H | H | 7-Cl | H |
| W.231 | B₄ | CH₃ | H | F | H | 7-Cl | H |
| W.232 | B₄ | CH₃ | CH₃ | F | H | 7-Cl | H |
| W.233 | B₄ | CH₃ | H | F | F | 7-Cl | H |
| W.234 | B₄ | CH₃ | H | CH₃ | CH₃ | 7-Cl | H |
| W.235 | B₄ | H | H | H | H | 8-Cl | H |
| W.236 | B₄ | CH₃ | H | H | H | 8-Cl | H |
| W.237 | B₄ | CH₂CH₃ | H | H | H | 8-Cl | H |
| W.238 | B₄ | CH₃ | CH₃ | H | H | 8-Cl | H |
| W.239 | B₄ | CH₃ | CH₂CH₃ | H | H | 8-Cl | H |
| W.240 | B₄ | CH₃ | H | F | H | 8-Cl | H |
| W.241 | B₄ | CH₃ | CH₃ | F | H | 8-Cl | H |
| W.242 | B₄ | CH₃ | H | F | F | 8-Cl | H |
| W.243 | B₄ | CH₃ | H | CH₃ | CH₃ | 8-Cl | H |
| W.244 | B₄ | H | H | H | H | 2-Cl | 4-Cl |
| W.245 | B₄ | CH₃ | H | H | H | 2-Cl | 4-Cl |
| W.246 | B₄ | CH₂CH₃ | H | H | H | 2-Cl | 4-Cl |
| W.247 | B₄ | CH₃ | CH₃ | H | H | 2-Cl | 4-Cl |
| W.248 | B₄ | CH₃ | CH₂CH₃ | H | H | 2-Cl | 4-Cl |
| W.249 | B₄ | CH₃ | H | F | H | 2-Cl | 4-Cl |
| W.250 | B₄ | CH₃ | CH₃ | F | H | 2-Cl | 4-Cl |
| W.251 | B₄ | CH₃ | H | F | F | 2-Cl | 4-Cl |
| W.252 | B₄ | CH₃ | H | CH₃ | CH₃ | 2-Cl | 4-Cl |
| W.253 | B₄ | H | H | H | H | 2-Cl | 6-Cl |
| W.254 | B₄ | CH₃ | H | H | H | 2-Cl | 6-Cl |
| W.255 | B₄ | CH₂CH₃ | H | H | H | 2-Cl | 6-Cl |
| W.256 | B₄ | CH₃ | CH₃ | H | H | 2-Cl | 6-Cl |
| W.257 | B₄ | CH₃ | CH₂CH₃ | H | H | 2-Cl | 6-Cl |
| W.258 | B₄ | CH₃ | H | F | H | 2-Cl | 6-Cl |
| W.259 | B₄ | CH₃ | CH₃ | F | H | 2-Cl | 6-Cl |
| W.260 | B₄ | CH₃ | H | F | F | 2-Cl | 6-Cl |
| W.261 | B₄ | CH₃ | H | CH₃ | CH₃ | 2-Cl | 6-Cl |
| W.262 | B₄ | H | H | H | H | 2-Cl | 8-Cl |
| W.263 | B₄ | CH₃ | H | H | H | 2-Cl | 8-Cl |
| W.264 | B₄ | CH₂CH₃ | H | H | H | 2-Cl | 8-Cl |
| W.265 | B₄ | CH₃ | CH₃ | H | H | 2-Cl | 8-Cl |
| W.266 | B₄ | CH₃ | CH₂CH₃ | H | H | 2-Cl | 8-Cl |
| W.267 | B₄ | CH₃ | H | F | H | 2-Cl | 8-Cl |
| W.268 | B₄ | CH₃ | CH₃ | F | H | 2-Cl | 8-Cl |
| W.269 | B₄ | CH₃ | H | F | F | 2-Cl | 8-Cl |
| W.270 | B₄ | CH₃ | H | CH₃ | CH₃ | 2-Cl | 8-Cl |
| W.271 | B₄ | H | H | H | H | 6-p-Cl-phenyl | 2-Cl |
| W.272 | B₄ | CH₃ | H | H | H | 6-p-Cl-phenyl | 2-Cl |
| W.273 | B₄ | CH₂CH₃ | H | H | H | 6-p-Cl-phenyl | 2-Cl |
| W.274 | B₄ | CH₃ | CH₃ | H | H | 6-p-Cl-phenyl | 2-Cl |
| W.275 | B₄ | CH₃ | CH₂CH₃ | H | H | 6-p-Cl-phenyl | 2-Cl |
| W.276 | B₄ | CH₃ | H | F | H | 6-p-Cl-phenyl | 2-Cl |
| W.277 | B₄ | CH₃ | CH₃ | F | H | 6-p-Cl-phenyl | 2-Cl |
| W.278 | B₄ | CH₃ | H | F | F | 6-p-Cl-phenyl | 2-Cl |
| W.279 | B₄ | CH₃ | H | CH₃ | CH₃ | 6-p-Cl-phenyl | 2-Cl |
| W.280 | B₄ | H | H | H | H | 8-p-Cl-phenyl | 2-Cl |
| W.281 | B₄ | CH₃ | H | H | H | 8-p-Cl-phenyl | 2-Cl |
| W.282 | B₄ | CH₂CH₃ | H | H | H | 8-p-Cl-phenyl | 2-Cl |
| W.283 | B₄ | CH₃ | CH₃ | H | H | 8-p-Cl-phenyl | 2-Cl |
| W.284 | B₄ | CH₃ | CH₂CH₃ | H | H | 8-p-Cl-phenyl | 2-Cl |
| W.285 | B₄ | CH₃ | H | F | H | 8-p-Cl-phenyl | 2-Cl |
| W.286 | B₄ | CH₃ | CH₃ | F | H | 8-p-Cl-phenyl | 2-Cl |
| W.287 | B₄ | CH₃ | H | F | F | 8-p-Cl-phenyl | 2-Cl |
| W.288 | B₄ | CH₃ | H | CH₃ | CH₃ | 8-p-Cl-phenyl | 2-Cl |

Table 8 provides 288 compounds of formula (IB), wherein A is

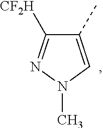

wherein the broken lines indicate the point of attachment of the group A to the amide group, and B, R₁, R₂, R₃, R₄, R₉ₐ and R_{9b} are as defined in Table W. For example, compound 7.001 has the following structure:

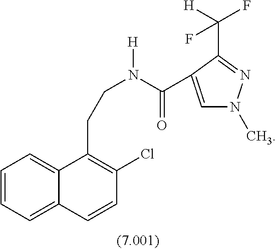

Table 9 provides 288 compounds of formula (IB) wherein A is

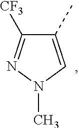

wherein the broken lines indicate the point of attachment of the group A to the amide group, and B, R₁, R₂, R₃, R₄, R₉ₐ and R_{9b} are as defined in Table W.

Table 10 provides 288 compounds of formula (IB) wherein A is

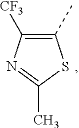

wherein the broken lines indicate the point of attachment of the group A to the amide group, and B, R₁, R₂, R₃, R₄, R₉ₐ and R_{9b} are as defined in Table W.

Table 11 provides 288 compounds of formula (IB) wherein A is

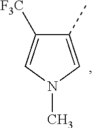

wherein the broken lines indicate the point of attachment of the group A to the amide group, and B, R₁, R₂, R₃, R₄, R₉ₐ and R_{9b} are as defined in Table W.

Table 12 provides 288 compounds of formula (IB) wherein A is

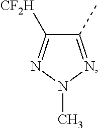

wherein the broken lines indicate the point of attachment of the group A to the amide group, and B, R₁, R₂, R₃, R₄, R₉ₐ and R_{9b} are as defined in Table W.

### Table 13: Compounds of Formula IIA

Illustrative of the compounds of formula (IIA) are the compounds listed in Table 12 below. Characterising data for these compounds are given in Table 14.

TABLE 13
(IIA)

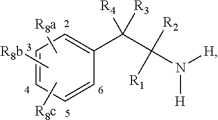

| Comp. No. | R₁ | R₂ | R₃ | R₄ | R₈ₐ | R_{8b} | R_{8c} |
|---|---|---|---|---|---|---|---|
| Z1.001 | H | H | H | H | 4-Cl | H | H |
| Z1.002 | CH₃ | H | H | H | 4-Cl | H | H |
| Z1.003 | CH₂CH₃ | H | H | H | 4-Cl | H | H |
| Z1.004 | (CH₂)₂CH₃ | H | H | H | 4-Cl | H | H |
| Z1.005 | CH(CH₃)₂ | H | H | H | 4-Cl | H | H |
| Z1.006 | F | H | H | H | 4-Cl | H | H |
| Z1.007 | H | H | CH₃ | H | 4-Cl | H | H |
| Z1.008 | H | H | CH₂CH₃ | H | 4-Cl | H | H |
| Z1.009 | H | H | (CH₂)₂CH₃ | H | 4-Cl | H | H |
| Z1.010 | H | H | CH(CH₃)₂ | H | 4-Cl | H | H |
| Z1.011 | H | H | F | H | 4-Cl | H | H |
| Z1.012 | CH₃ | CH₃ | H | H | 4-Cl | H | H |
| Z1.013 | CH₂CH₃ | CH₃ | H | H | 4-Cl | H | H |
| Z1.014 | (CH₂)₂CH₃ | CH₃ | H | H | 4-Cl | H | H |
| Z1.015 | CH(CH₃)₂ | CH₃ | H | H | 4-Cl | H | H |
| Z1.016 | F | CH₃ | H | H | 4-Cl | H | H |
| Z1.017 | CH₃ | H | CH₃ | H | 4-Cl | H | H |
| Z1.018 | CH₂CH₃ | H | CH₃ | H | 4-Cl | H | H |
| Z1.019 | (CH₂)₂CH₃ | H | CH₃ | H | 4-Cl | H | H |
| Z1.020 | CH(CH₃)₂ | H | CH₃ | H | 4-Cl | H | H |
| Z1.021 | CH₃ | H | F | H | 4-Cl | H | H |
| Z1.022 | CH₂CH₃ | H | F | H | 4-Cl | H | H |
| Z1.023 | H | H | CH₃ | CH₃ | 4-Cl | H | H |
| Z1.024 | H | H | CH₂CH₃ | CH₃ | 4-Cl | H | H |
| Z1.025 | H | H | CH(CH₃)₂ | CH₃ | 4-Cl | H | H |
| Z1.026 | H | H | CH₃ | F | 4-Cl | H | H |
| Z1.027 | H | H | CH₂CH₃ | F | 4-Cl | H | H |
| Z1.028 | H | H | (CH₂)₂CH₃ | F | 4-Cl | H | H |
| Z1.029 | H | H | CH(CH₃)₂ | F | 4-Cl | H | H |
| Z1.030 | H | H | F | F | 4-Cl | H | H |
| Z1.031 | CH₃ | H | CH₃ | F | 4-Cl | H | H |
| Z1.032 | CH₃ | H | CH₂CH₃ | F | 4-Cl | H | H |
| Z1.033 | CH₃ | H | (CH₂)₂CH₃ | F | 4-Cl | H | H |
| Z1.034 | CH₃ | H | CH(CH₃)₂ | F | 4-Cl | H | H |
| Z1.035 | CH₃ | H | F | F | 4-Cl | H | H |
| Z1.036 | (CH₂)₂ | | H | H | 4-Cl | H | H |
| Z1.037 | (CH₂)₂ | | CH₃ | H | 4-Cl | H | H |
| Z1.038 | (CH₂)₂ | | (CH₂)₂CH₃ | H | 4-Cl | H | H |
| Z1.039 | (CH₂)₂ | | CH(CH₃)₂ | H | 4-Cl | H | H |
| Z1.040 | H | H | H | H | 4-CF₃ | H | H |
| Z1.041 | CH₃ | H | H | H | 4-CF₃ | H | H |
| Z1.042 | CH₂CH₃ | H | H | H | 4-CF₃ | H | H |
| Z1.043 | (CH₂)₂CH₃ | H | H | H | 4-CF₃ | H | H |
| Z1.044 | CH(CH₃)₂ | H | H | H | 4-CF₃ | H | H |
| Z1.045 | F | H | H | H | 4-CF₃ | H | H |
| Z1.046 | H | H | CH₃ | H | 4-CF₃ | H | H |
| Z1.047 | H | H | CH₂CH₃ | H | 4-CF₃ | H | H |
| Z1.048 | H | H | (CH₂)₂CH₃ | H | 4-CF₃ | H | H |
| Z1.049 | H | H | CH(CH₃)₂ | H | 4-CF₃ | H | H |
| Z1.050 | H | H | F | H | 4-CF₃ | H | H |
| Z1.051 | CH₃ | CH₃ | H | H | 4-CF₃ | H | H |
| Z1.052 | CH₂CH₃ | CH₃ | H | H | 4-CF₃ | H | H |
| Z1.053 | (CH₂)₂CH₃ | CH₃ | H | H | 4-CF₃ | H | H |
| Z1.054 | CH(CH₃)₂ | CH₃ | H | H | 4-CF₃ | H | H |
| Z1.055 | F | CH₃ | H | H | 4-CF₃ | H | H |
| Z1.056 | CH₃ | H | CH₃ | H | 4-CF₃ | H | H |
| Z1.057 | CH₂CH₃ | H | CH₃ | H | 4-CF₃ | H | H |
| Z1.058 | (CH₂)₂CH₃ | H | CH₃ | H | 4-CF₃ | H | H |
| Z1.059 | CH(CH₃)₂ | H | CH₃ | H | 4-CF₃ | H | H |
| Z1.060 | CH₃ | H | F | H | 4-CF₃ | H | H |
| Z1.061 | CH₂CH₃ | H | F | H | 4-CF₃ | H | H |
| Z1.062 | H | H | CH₃ | CH₃ | 4-CF₃ | H | H |
| Z1.063 | H | H | CH₂CH₃ | CH₃ | 4-CF₃ | H | H |
| Z1.064 | H | H | CH(CH₃)₂ | CH₃ | 4-CF₃ | H | H |
| Z1.065 | H | H | CH₃ | F | 4-CF₃ | H | H |
| Z1.066 | H | H | CH₂CH₃ | F | 4-CF₃ | H | H |
| Z1.067 | H | H | (CH₂)₂CH₃ | F | 4-CF₃ | H | H |
| Z1.068 | H | H | CH(CH₃)₂ | F | 4-CF₃ | H | H |
| Z1.069 | H | H | F | F | 4-CF₃ | H | H |
| Z1.070 | CH₃ | H | CH₃ | F | 4-CF₃ | H | H |
| Z1.071 | CH₃ | H | CH₂CH₃ | F | 4-CF₃ | H | H |
| Z1.072 | CH₃ | H | (CH₂)₂CH₃ | F | 4-CF₃ | H | H |
| Z1.073 | CH₃ | H | CH(CH₃)₂ | F | 4-CF₃ | H | H |
| Z1.074 | CH₃ | H | F | F | 4-CF₃ | H | H |
| Z1.075 | (CH₂)₂ | | H | H | 4-CF₃ | H | H |
| Z1.076 | (CH₂)₂ | | CH₃ | H | 4-CF₃ | H | H |
| Z1.077 | (CH₂)₂ | | (CH₂)₂CH₃ | H | 4-CF₃ | H | H |
| Z1.078 | (CH₂)₂ | | CH(CH₃)₂ | H | 4-CF₃ | H | H |
| Z1.079 | H | H | H | H | 4-OCF₃ | H | H |
| Z1.080 | CH₃ | H | H | H | 4-OCF₃ | H | H |
| Z1.081 | CH₂CH₃ | H | H | H | 4-OCF₃ | H | H |
| Z1.082 | (CH₂)₂CH₃ | H | H | H | 4-OCF₃ | H | H |
| Z1.083 | CH(CH₃)₂ | H | H | H | 4-OCF₃ | H | H |
| Z1.084 | F | H | H | H | 4-OCF₃ | H | H |
| Z1.085 | H | H | CH₃ | H | 4-OCF₃ | H | H |
| Z1.086 | H | H | CH₂CH₃ | H | 4-OCF₃ | H | H |
| Z1.087 | H | H | (CH₂)₂CH₃ | H | 4-OCF₃ | H | H |
| Z1.088 | H | H | CH(CH₃)₂ | H | 4-OCF₃ | H | H |
| Z1.089 | H | H | F | H | 4-OCF₃ | H | H |
| Z1.090 | CH₃ | CH₃ | H | H | 4-OCF₃ | H | H |
| Z1.091 | CH₂CH₃ | CH₃ | H | H | 4-OCF₃ | H | H |
| Z1.092 | (CH₂)₂CH₃ | CH₃ | H | H | 4-OCF₃ | H | H |
| Z1.093 | CH(CH₃)₂ | CH₃ | H | H | 4-OCF₃ | H | H |
| Z1.094 | F | CH₃ | H | H | 4-OCF₃ | H | H |
| Z1.095 | CH₃ | H | CH₃ | H | 4-OCF₃ | H | H |
| Z1.096 | CH₂CH₃ | H | CH₃ | H | 4-OCF₃ | H | H |
| Z1.097 | (CH₂)₂CH₃ | H | CH₃ | H | 4-OCF₃ | H | H |
| Z1.098 | CH(CH₃)₂ | H | CH₃ | H | 4-OCF₃ | H | H |
| Z1.099 | CH₃ | H | F | H | 4-OCF₃ | H | H |
| Z1.100 | CH₂CH₃ | H | F | H | 4-OCF₃ | H | H |
| Z1.101 | H | H | CH₃ | CH₃ | 4-OCF₃ | H | H |
| Z1.102 | H | H | CH₂CH₃ | CH₃ | 4-OCF₃ | H | H |
| Z1.103 | H | H | CH(CH₃)₂ | CH₃ | 4-OCF₃ | H | H |
| Z1.104 | H | H | CH₃ | F | 4-OCF₃ | H | H |
| Z1.105 | H | H | CH₂CH₃ | F | 4-OCF₃ | H | H |
| Z1.106 | H | H | (CH₂)₂CH₃ | F | 4-OCF₃ | H | H |
| Z1.107 | H | H | CH(CH₃)₂ | F | 4-OCF₃ | H | H |
| Z1.108 | H | H | F | F | 4-OCF₃ | H | H |
| Z1.109 | CH₃ | H | CH₃ | F | 4-OCF₃ | H | H |
| Z1.110 | CH₃ | H | CH₂CH₃ | F | 4-OCF₃ | H | H |
| Z1.111 | CH₃ | H | (CH₂)₂CH₃ | F | 4-OCF₃ | H | H |
| Z1.112 | CH₃ | H | CH(CH₃)₂ | F | 4-OCF₃ | H | H |
| Z1.113 | CH₃ | H | F | F | 4-OCF₃ | H | H |
| Z1.114 | (CH₂)₂ | | H | H | 4-OCF₃ | H | H |
| Z1.115 | (CH₂)₂ | | CH₃ | H | 4-OCF₃ | H | H |
| Z1.116 | (CH₂)₂ | | (CH₂)₂CH₃ | H | 4-OCF₃ | H | H |
| Z1.117 | (CH₂)₂ | | CH(CH₃)₂ | H | 4-OCF₃ | H | H |
| Z1.118 | H | H | H | H | 4-C≡CC(CH₃)₃ | H | H |
| Z1.119 | CH₃ | H | H | H | 4-C≡CC(CH₃)₃ | H | H |
| Z1.120 | CH₂CH₃ | H | H | H | 4-C≡CC(CH₃)₃ | H | H |
| Z1.121 | (CH₂)₂CH₃ | H | H | H | 4-C≡CC(CH₃)₃ | H | H |
| Z1.122 | CH(CH₃)₂ | H | H | H | 4-C≡CC(CH₃)₃ | H | H |
| Z1.123 | F | H | H | H | 4-C≡CC(CH₃)₃ | H | H |
| Z1.124 | H | H | CH₃ | H | 4-C≡CC(CH₃)₃ | H | H |
| Z1.125 | H | H | CH₂CH₃ | H | 4-C≡CC(CH₃)₃ | H | H |
| Z1.126 | H | H | (CH₂)₂CH₃ | H | 4-C≡CC(CH₃)₃ | H | H |
| Z1.127 | H | H | CH(CH₃)₂ | H | 4-C≡CC(CH₃)₃ | H | H |
| Z1.128 | H | H | F | H | 4-C≡CC(CH₃)₃ | H | H |
| Z1.129 | CH₃ | CH₃ | H | H | 4-C≡CC(CH₃)₃ | H | H |
| Z1.130 | CH₂CH₃ | CH₃ | H | H | 4-C≡CC(CH₃)₃ | H | H |
| Z1.131 | (CH₂)₂CH₃ | CH₃ | H | H | 4-C≡CC(CH₃)₃ | H | H |
| Z1.132 | CH(CH₃)₂ | CH₃ | H | H | 4-C≡CC(CH₃)₃ | H | H |
| Z1.133 | F | CH₃ | H | H | 4-C≡CC(CH₃)₃ | H | H |
| Z1.134 | CH₃ | H | CH₃ | H | 4-C≡CC(CH₃)₃ | H | H |
| Z1.135 | CH₂CH₃ | H | CH₃ | H | 4-C≡CC(CH₃)₃ | H | H |
| Z1.136 | (CH₂)₂CH₃ | H | CH₃ | H | 4-C≡CC(CH₃)₃ | H | H |
| Z1.137 | CH(CH₃)₂ | H | CH₃ | H | 4-C≡CC(CH₃)₃ | H | H |
| Z1.138 | CH₃ | H | F | H | 4-C≡CC(CH₃)₃ | H | H |
| Z1.139 | CH₂CH₃ | H | F | H | 4-C≡CC(CH₃)₃ | H | H |
| Z1.140 | H | H | CH₃ | CH₃ | 4-C≡CC(CH₃)₃ | H | H |
| Z1.141 | H | H | CH₂CH₃ | CH₃ | 4-C≡CC(CH₃)₃ | H | H |
| Z1.142 | H | H | CH(CH₃)₂ | CH₃ | 4-C≡CC(CH₃)₃ | H | H |
| Z1.143 | H | H | CH₃ | F | 4-C≡CC(CH₃)₃ | H | H |
| Z1.144 | H | H | CH₂CH₃ | F | 4-C≡CC(CH₃)₃ | H | H |
| Z1.145 | H | H | (CH₂)₂CH₃ | F | 4-C≡CC(CH₃)₃ | H | H |
| Z1.146 | H | H | CH(CH₃)₂ | F | 4-C≡CC(CH₃)₃ | H | H |
| Z1.147 | H | H | F | F | 4-C≡CC(CH₃)₃ | H | H |
| Z1.148 | CH₃ | H | CH₃ | F | 4-C≡CC(CH₃)₃ | H | H |
| Z1.149 | CH₃ | H | CH₂CH₃ | F | 4-C≡CC(CH₃)₃ | H | H |
| Z1.150 | CH₃ | H | (CH₂)₂CH₃ | F | 4-C≡CC(CH₃)₃ | H | H |
| Z1.151 | CH₃ | H | CH(CH₃)₂ | F | 4-C≡CC(CH₃)₃ | H | H |
| Z1.152 | CH₃ | H | F | F | 4-C≡CC(CH₃)₃ | H | H |
| Z1.153 | (CH₂)₂ | | H | H | 4-C≡CC(CH₃)₃ | H | H |
| Z1.154 | (CH₂)₂ | | CH₃ | H | 4-C≡CC(CH₃)₃ | H | H |
| Z1.155 | (CH₂)₂ | | (CH₂)₂CH₃ | H | 4-C≡CC(CH₃)₃ | H | H |
| Z1.156 | (CH₂)₂ | | CH(CH₃)₂ | H | 4-C≡CC(CH₃)₃ | H | H |
| Z1.157 | H | H | H | H | 4-p-Cl-phenyl | H | H |
| Z1.158 | CH₃ | H | H | H | 4-p-Cl-phenyl | H | H |
| Z1.159 | CH₂CH₃ | H | H | H | 4-p-Cl-phenyl | H | H |
| Z1.160 | (CH₂)₂CH₃ | H | H | H | 4-p-Cl-phenyl | H | H |
| Z1.161 | CH(CH₃)₂ | H | H | H | 4-p-Cl-phenyl | H | H |
| Z1.162 | F | H | H | H | 4-p-Cl-phenyl | H | H |
| Z1.163 | H | H | CH₃ | H | 4-p-Cl-phenyl | H | H |
| Z1.164 | H | H | CH₂CH₃ | H | 4-p-Cl-phenyl | H | H |
| Z1.165 | H | H | (CH₂)₂CH₃ | H | 4-p-Cl-phenyl | H | H |
| Z1.166 | H | H | CH(CH₃)₂ | H | 4-p-Cl-phenyl | H | H |
| Z1.167 | H | H | F | H | 4-p-Cl-phenyl | H | H |
| Z1.168 | CH₃ | CH₃ | H | H | 4-p-Cl-phenyl | H | H |
| Z1.169 | CH₂CH₃ | CH₃ | H | H | 4-p-Cl-phenyl | H | H |
| Z1.170 | (CH₂)₂CH₃ | CH₃ | H | H | 4-p-Cl-phenyl | H | H |
| Z1.171 | CH(CH₃)₂ | CH₃ | H | H | 4-p-Cl-phenyl | H | H |
| Z1.172 | F | CH₃ | H | H | 4-p-Cl-phenyl | H | H |
| Z1.173 | CH₃ | H | CH₃ | H | 4-p-Cl-phenyl | H | H |
| Z1.174 | CH₂CH₃ | H | CH₃ | H | 4-p-Cl-phenyl | H | H |
| Z1.175 | (CH₂)₂CH₃ | H | CH₃ | H | 4-p-Cl-phenyl | H | H |
| Z1.176 | CH(CH₃)₂ | H | CH₃ | H | 4-p-Cl-phenyl | H | H |
| Z1.177 | CH₃ | H | F | H | 4-p-Cl-phenyl | H | H |
| Z1.178 | CH₂CH₃ | H | F | H | 4-p-Cl-phenyl | H | H |
| Z1.179 | H | H | CH₃ | CH₃ | 4-p-Cl-phenyl | H | H |
| Z1.180 | H | H | CH₂CH₃ | CH₃ | 4-p-Cl-phenyl | H | H |
| Z1.181 | H | H | CH(CH₃)₂ | CH₃ | 4-p-Cl-phenyl | H | H |
| Z1.182 | H | H | CH₃ | F | 4-p-Cl-phenyl | H | H |
| Z1.183 | H | H | CH₂CH₃ | F | 4-p-Cl-phenyl | H | H |
| Z1.184 | H | H | (CH₂)₂CH₃ | F | 4-p-Cl-phenyl | H | H |
| Z1.185 | H | H | CH(CH₃)₂ | F | 4-p-Cl-phenyl | H | H |
| Z1.186 | H | H | F | F | 4-p-Cl-phenyl | H | H |
| Z1.187 | CH₃ | H | CH₃ | F | 4-p-Cl-phenyl | H | H |
| Z1.188 | CH₃ | H | CH₂CH₃ | F | 4-p-Cl-phenyl | H | H |
| Z1.189 | CH₃ | H | (CH₂)₂CH₃ | F | 4-p-Cl-phenyl | H | H |
| Z1.190 | CH₃ | H | CH(CH₃)₂ | F | 4-p-Cl-phenyl | H | H |
| Z1.191 | CH₃ | H | F | F | 4-p-Cl-phenyl | H | H |
| Z1.192 | (CH₂)₂ | | H | H | 4-p-Cl-phenyl | H | H |
| Z1.193 | (CH₂)₂ | | CH₃ | H | 4-p-Cl-phenyl | H | H |
| Z1.194 | (CH₂)₂ | | (CH₂)₂CH₃ | H | 4-p-Cl-phenyl | H | H |
| Z1.195 | (CH₂)₂ | | CH(CH₃)₂ | H | 4-p-Cl-phenyl | H | H |
| Z1.196 | H | H | H | H | 2-Cl | 4-Cl | H |
| Z1.197 | CH₃ | H | H | H | 2-Cl | 4-Cl | H |
| Z1.198 | CH₂CH₃ | H | H | H | 2-Cl | 4-Cl | H |
| Z1.199 | (CH₂)₂CH₃ | H | H | H | 2-Cl | 4-Cl | H |
| Z1.200 | CH(CH₃)₂ | H | H | H | 2-Cl | 4-Cl | H |
| Z1.201 | F | H | H | H | 2-Cl | 4-Cl | H |
| Z1.202 | H | H | CH₃ | H | 2-Cl | 4-Cl | H |
| Z1.203 | H | H | CH₂CH₃ | H | 2-Cl | 4-Cl | H |
| Z1.204 | H | H | (CH₂)₂CH₃ | H | 2-Cl | 4-Cl | H |
| Z1.205 | H | H | CH(CH₃)₂ | H | 2-Cl | 4-Cl | H |
| Z1.206 | H | H | F | H | 2-Cl | 4-Cl | H |
| Z1.207 | CH₃ | CH₃ | H | H | 2-Cl | 4-Cl | H |
| Z1.208 | CH₂CH₃ | CH₃ | H | H | 2-Cl | 4-Cl | H |
| Z1.209 | (CH₂)₂CH₃ | CH₃ | H | H | 2-Cl | 4-Cl | H |
| Z1.210 | CH(CH₃)₂ | CH₃ | H | H | 2-Cl | 4-Cl | H |
| Z1.211 | F | CH₃ | H | H | 2-Cl | 4-Cl | H |
| Z1.212 | CH₃ | H | CH₃ | H | 2-Cl | 4-Cl | H |
| Z1.213 | CH₂CH₃ | H | CH₃ | H | 2-Cl | 4-Cl | H |
| Z1.214 | (CH₂)₂CH₃ | H | CH₃ | H | 2-Cl | 4-Cl | H |
| Z1.215 | CH(CH₃)₂ | H | CH₃ | H | 2-Cl | 4-Cl | H |
| Z1.216 | CH₃ | H | F | H | 2-Cl | 4-Cl | H |
| Z1.217 | CH₂CH₃ | H | F | H | 2-Cl | 4-Cl | H |
| Z1.218 | H | H | CH₃ | CH₃ | 2-Cl | 4-Cl | H |
| Z1.219 | H | H | CH₂CH₃ | CH₃ | 2-Cl | 4-Cl | H |
| Z1.220 | H | H | CH(CH₃)₂ | CH₃ | 2-Cl | 4-Cl | H |
| Z1.221 | H | H | CH₃ | F | 2-Cl | 4-Cl | H |
| Z1.222 | H | H | CH₂CH₃ | F | 2-Cl | 4-Cl | H |
| Z1.223 | H | H | (CH₂)₂CH₃ | F | 2-Cl | 4-Cl | H |
| Z1.224 | H | H | CH(CH₃)₂ | F | 2-Cl | 4-Cl | H |
| Z1.225 | H | H | F | F | 2-Cl | 4-Cl | H |
| Z1.226 | CH₃ | H | CH₃ | F | 2-Cl | 4-Cl | H |
| Z1.227 | CH₃ | H | CH₂CH₃ | F | 2-Cl | 4-Cl | H |
| Z1.228 | CH₃ | H | (CH₂)₂CH₃ | F | 2-Cl | 4-Cl | H |
| Z1.229 | CH₃ | H | CH(CH₃)₂ | F | 2-Cl | 4-Cl | H |
| Z1.230 | CH₃ | H | F | F | 2-Cl | 4-Cl | H |
| Z1.231 | (CH₂)₂ | | H | H | 2-Cl | 4-Cl | H |
| Z1.232 | (CH₂)₂ | | CH₃ | H | 2-Cl | 4-Cl | H |
| Z1.233 | (CH₂)₂ | | (CH₂)₂CH₃ | H | 2-Cl | 4-Cl | H |
| Z1.234 | (CH₂)₂ | | CH(CH₃)₂ | H | 2-Cl | 4-Cl | H |
| Z1.235 | H | H | H | H | 3-Cl | 4-Cl | H |
| Z1.236 | CH₃ | H | H | H | 3-Cl | 4-Cl | H |
| Z1.237 | CH₂CH₃ | H | H | H | 3-Cl | 4-Cl | H |
| Z1.238 | (CH₂)₂CH₃ | H | H | H | 3-Cl | 4-Cl | H |
| Z1.239 | CH(CH₃)₂ | H | H | H | 3-Cl | 4-Cl | H |
| Z1.240 | F | H | H | H | 3-Cl | 4-Cl | H |
| Z1.241 | H | H | CH₃ | H | 3-Cl | 4-Cl | H |
| Z1.242 | H | H | CH₂CH₃ | H | 3-Cl | 4-Cl | H |
| Z1.243 | H | H | (CH₂)₂CH₃ | H | 3-Cl | 4-Cl | H |
| Z1.244 | H | H | CH(CH₃)₂ | H | 3-Cl | 4-Cl | H |
| Z1.245 | H | H | F | H | 3-Cl | 4-Cl | H |
| Z1.246 | CH₃ | CH₃ | H | H | 3-Cl | 4-Cl | H |
| Z1.247 | CH₂CH₃ | CH₃ | H | H | 3-Cl | 4-Cl | H |
| Z1.248 | (CH₂)₂CH₃ | CH₃ | H | H | 3-Cl | 4-Cl | H |
| Z1.249 | CH(CH₃)₂ | CH₃ | H | H | 3-Cl | 4-Cl | H |
| Z1.250 | F | CH₃ | H | H | 3-Cl | 4-Cl | H |
| Z1.251 | CH₃ | H | CH₃ | H | 3-Cl | 4-Cl | H |
| Z1.252 | CH₂CH₃ | H | CH₃ | H | 3-Cl | 4-Cl | H |
| Z1.253 | (CH₂)₂CH₃ | H | CH₃ | H | 3-Cl | 4-Cl | H |
| Z1.254 | CH(CH₃)₂ | H | CH₃ | H | 3-Cl | 4-Cl | H |
| Z1.255 | CH₃ | H | F | H | 3-Cl | 4-Cl | H |
| Z1.256 | CH₂CH₃ | H | F | H | 3-Cl | 4-Cl | H |
| Z1.247 | H | H | CH₃ | CH₃ | 3-Cl | 4-Cl | H |
| Z1.258 | H | H | CH₂CH₃ | CH₃ | 3-Cl | 4-Cl | H |
| Z1.259 | H | H | CH(CH₃)₂ | CH₃ | 3-Cl | 4-Cl | H |
| Z1.260 | H | H | CH₃ | F | 3-Cl | 4-Cl | H |
| Z1.261 | H | H | CH₂CH₃ | F | 3-Cl | 4-Cl | H |
| Z1.262 | H | H | (CH₂)₂CH₃ | F | 3-Cl | 4-Cl | H |
| Z1.263 | H | H | CH(CH₃)₂ | F | 3-Cl | 4-Cl | H |
| Z1.264 | H | H | F | F | 3-Cl | 4-Cl | H |
| Z1.265 | CH₃ | H | CH₃ | F | 3-Cl | 4-Cl | H |
| Z1.266 | CH₃ | H | CH₂CH₃ | F | 3-Cl | 4-Cl | H |
| Z1.267 | CH₃ | H | (CH₂)₂CH₃ | F | 3-Cl | 4-Cl | H |
| Z1.268 | CH₃ | H | CH(CH₃)₂ | F | 3-Cl | 4-Cl | H |
| Z1.269 | CH₃ | H | F | F | 3-Cl | 4-Cl | H |
| Z1.270 | (CH₂)₂ | | H | H | 3-Cl | 4-Cl | H |
| Z1.271 | (CH₂)₂ | | CH₃ | H | 3-Cl | 4-Cl | H |
| Z1.272 | (CH₂)₂ | | (CH₂)₂CH₃ | H | 3-Cl | 4-Cl | H |
| Z1.273 | (CH₂)₂ | | CH(CH₃)₂ | H | 3-Cl | 4-Cl | H |
| Z1.274 | H | H | H | H | 2-F | 4-Cl | H |
| Z1.275 | CH₃ | H | H | H | 2-F | 4-Cl | H |
| Z1.276 | CH₂CH₃ | H | H | H | 2-F | 4-Cl | H |
| Z1.277 | (CH₂)₂CH₃ | H | H | H | 2-F | 4-Cl | H |
| Z1.278 | CH(CH₃)₂ | H | H | H | 2-F | 4-Cl | H |
| Z1.279 | F | H | H | H | 2-F | 4-Cl | H |
| Z1.280 | H | H | CH₃ | H | 2-F | 4-Cl | H |
| Z1.281 | H | H | CH₂CH₃ | H | 2-F | 4-Cl | H |
| Z1.282 | H | H | (CH₂)₂CH₃ | H | 2-F | 4-Cl | H |
| Z1.283 | H | H | CH(CH₃)₂ | H | 2-F | 4-Cl | H |
| Z1.284 | H | H | F | H | 2-F | 4-Cl | H |
| Z1.285 | CH₃ | CH₃ | H | H | 2-F | 4-Cl | H |
| Z1.286 | CH₂CH₃ | CH₃ | H | H | 2-F | 4-Cl | H |
| Z1.287 | (CH₂)₂CH₃ | CH₃ | H | H | 2-F | 4-Cl | H |
| Z1.288 | CH(CH₃)₂ | CH₃ | H | H | 2-F | 4-Cl | H |
| Z1.289 | F | CH₃ | H | H | 2-F | 4-Cl | H |
| Z1.290 | CH₃ | H | CH₃ | H | 2-F | 4-Cl | H |
| Z1.291 | CH₂CH₃ | H | CH₃ | H | 2-F | 4-Cl | H |
| Z1.292 | (CH₂)₂CH₃ | H | CH₃ | H | 2-F | 4-Cl | H |
| Z1.293 | CH(CH₃)₂ | H | CH₃ | H | 2-F | 4-Cl | H |
| Z1.294 | CH₃ | H | F | H | 2-F | 4-Cl | H |
| Z1.295 | CH₂CH₃ | H | F | H | 2-F | 4-Cl | H |
| Z1.296 | H | H | CH₃ | CH₃ | 2-F | 4-Cl | H |
| Z1.297 | H | H | CH₂CH₃ | CH₃ | 2-F | 4-Cl | H |
| Z1.298 | H | H | CH(CH₃)₂ | CH₃ | 2-F | 4-Cl | H |
| Z1.299 | H | H | CH₃ | F | 2-F | 4-Cl | H |
| Z1.300 | H | H | CH₂CH₃ | F | 2-F | 4-Cl | H |
| Z1.301 | H | H | (CH₂)₂CH₃ | F | 2-F | 4-Cl | H |
| Z1.302 | H | H | CH(CH₃)₂ | F | 2-F | 4-Cl | H |
| Z1.303 | H | H | F | F | 2-F | 4-Cl | H |
| Z1.304 | CH₃ | H | CH₃ | F | 2-F | 4-Cl | H |
| Z1.305 | CH₃ | H | CH₂CH₃ | F | 2-F | 4-Cl | H |
| Z1.306 | CH₃ | H | (CH₂)₂CH₃ | F | 2-F | 4-Cl | H |
| Z1.307 | CH₃ | H | CH(CH₃)₂ | F | 2-F | 4-Cl | H |
| Z1.308 | CH₃ | H | F | F | 2-F | 4-Cl | H |
| Z1.309 | (CH₂)₂ | | H | H | 2-F | 4-Cl | H |
| Z1.310 | (CH₂)₂ | | CH₃ | H | 2-F | 4-Cl | H |
| Z1.311 | (CH₂)₂ | | (CH₂)₂CH₃ | H | 2-F | 4-Cl | H |
| Z1.312 | (CH₂)₂ | | CH(CH₃)₂ | H | 2-F | 4-Cl | H |
| Z1.313 | H | H | H | H | 4-F | 2-Cl | H |
| Z1.314 | CH₃ | H | H | H | 4-F | 2-Cl | H |
| Z1.315 | CH₂CH₃ | H | H | H | 4-F | 2-Cl | H |
| Z1.316 | (CH₂)₂CH₃ | H | H | H | 4-F | 2-Cl | H |
| Z1.317 | CH(CH₃)₂ | H | H | H | 4-F | 2-Cl | H |
| Z1.318 | F | H | H | H | 4-F | 2-Cl | H |
| Z1.319 | H | H | CH₃ | H | 4-F | 2-Cl | H |
| Z1.320 | H | H | CH₂CH₃ | H | 4-F | 2-Cl | H |
| Z1.321 | H | H | (CH₂)₂CH₃ | H | 4-F | 2-Cl | H |
| Z1.322 | H | H | CH(CH₃)₂ | H | 4-F | 2-Cl | H |
| Z1.323 | H | H | F | H | 4-F | 2-Cl | H |
| Z1.324 | CH₃ | CH₃ | H | H | 4-F | 2-Cl | H |
| Z1.325 | CH₂CH₃ | CH₃ | H | H | 4-F | 2-Cl | H |
| Z1.326 | (CH₂)₂CH₃ | CH₃ | H | H | 4-F | 2-Cl | H |
| Z1.327 | CH(CH₃)₂ | CH₃ | H | H | 4-F | 2-Cl | H |
| Z1.328 | F | CH₃ | H | H | 4-F | 2-Cl | H |
| Z1.329 | CH₃ | H | CH₃ | H | 4-F | 2-Cl | H |
| Z1.330 | CH₂CH₃ | H | CH₃ | H | 4-F | 2-Cl | H |
| Z1.331 | (CH₂)₂CH₃ | H | CH₃ | H | 4-F | 2-Cl | H |
| Z1.332 | CH(CH₃)₂ | H | CH₃ | H | 4-F | 2-Cl | H |
| Z1.333 | CH₃ | H | F | H | 4-F | 2-Cl | H |
| Z1.334 | CH₂CH₃ | H | F | H | 4-F | 2-Cl | H |
| Z1.335 | H | H | CH₃ | CH₃ | 4-F | 2-Cl | H |
| Z1.336 | H | H | CH₂CH₃ | CH₃ | 4-F | 2-Cl | H |
| Z1.337 | H | H | CH(CH₃)₂ | CH₃ | 4-F | 2-Cl | H |
| Z1.338 | H | H | CH₃ | F | 4-F | 2-Cl | H |
| Z1.339 | H | H | CH₂CH₃ | F | 4-F | 2-Cl | H |
| Z1.340 | H | H | (CH₂)₂CH₃ | F | 4-F | 2-Cl | H |
| Z1.341 | H | H | CH(CH₃)₂ | F | 4-F | 2-Cl | H |
| Z1.342 | H | H | F | F | 4-F | 2-Cl | H |
| Z1.343 | CH₃ | H | CH₃ | F | 4-F | 2-Cl | H |
| Z1.344 | CH₃ | H | CH₂CH₃ | F | 4-F | 2-Cl | H |
| Z1.345 | CH₃ | H | (CH₂)₂CH₃ | F | 4-F | 2-Cl | H |
| Z1.346 | CH₃ | H | CH(CH₃)₂ | F | 4-F | 2-Cl | H |
| Z1.347 | CH₃ | H | F | F | 4-F | 2-Cl | H |
| Z1.348 | (CH₂)₂ | | H | H | 4-F | 2-Cl | H |
| Z1.349 | (CH₂)₂ | | CH₃ | H | 4-F | 2-Cl | H |
| Z1.350 | (CH₂)₂ | | (CH₂)₂CH₃ | H | 4-F | 2-Cl | H |
| Z1.351 | (CH₂)₂ | | CH(CH₃)₂ | H | 4-F | 2-Cl | H |
| Z1.352 | H | H | H | H | 4-p-Cl-phenyl | 2-Cl | H |
| Z1.353 | CH₃ | H | H | H | 4-p-Cl-phenyl | 2-Cl | H |
| Z1.354 | CH₂CH₃ | H | H | H | 4-p-Cl-phenyl | 2-Cl | H |
| Z1.355 | (CH₂)₂CH₃ | H | H | H | 4-p-Cl-phenyl | 2-Cl | H |
| Z1.356 | CH(CH₃)₂ | H | H | H | 4-p-Cl-phenyl | 2-Cl | H |
| Z1.347 | F | H | H | H | 4-p-Cl-phenyl | 2-Cl | H |
| Z1.358 | H | H | CH₃ | H | 4-p-Cl-phenyl | 2-Cl | H |
| Z1.359 | H | H | CH₂CH₃ | H | 4-p-Cl-phenyl | 2-Cl | H |
| Z1.360 | H | H | (CH₂)₂CH₃ | H | 4-p-Cl-phenyl | 2-Cl | H |
| Z1.361 | H | H | CH(CH₃)₂ | H | 4-p-Cl-phenyl | 2-Cl | H |
| Z1.362 | H | H | F | H | 4-p-Cl-phenyl | 2-Cl | H |
| Z1.363 | CH₃ | CH₃ | H | H | 4-p-Cl-phenyl | 2-Cl | H |
| Z1.364 | CH₂CH₃ | CH₃ | H | H | 4-p-Cl-phenyl | 2-Cl | H |
| Z1.365 | (CH₂)₂CH₃ | CH₃ | H | H | 4-p-Cl-phenyl | 2-Cl | H |
| Z1.366 | CH(CH₃)₂ | CH₃ | H | H | 4-p-Cl-phenyl | 2-Cl | H |
| Z1.367 | F | CH₃ | H | H | 4-p-Cl-phenyl | 2-Cl | H |
| Z1.368 | CH₃ | H | CH₃ | H | 4-p-Cl-phenyl | 2-Cl | H |
| Z1.369 | CH₂CH₃ | H | CH₃ | H | 4-p-Cl-phenyl | 2-Cl | H |
| Z1.370 | (CH₂)₂CH₃ | H | CH₃ | H | 4-p-Cl-phenyl | 2-Cl | H |
| Z1.371 | CH(CH₃)₂ | H | CH₃ | H | 4-p-Cl-phenyl | 2-Cl | H |
| Z1.372 | CH₃ | H | F | H | 4-p-Cl-phenyl | 2-Cl | H |
| Z1.373 | CH₂CH₃ | H | F | H | 4-p-Cl-phenyl | 2-Cl | H |
| Z1.374 | H | H | CH₃ | CH₃ | 4-p-Cl-phenyl | 2-Cl | H |
| Z1.375 | H | H | CH₂CH₃ | CH₃ | 4-p-Cl-phenyl | 2-Cl | H |
| Z1.376 | H | H | CH(CH₃)₂ | CH₃ | 4-p-Cl-phenyl | 2-Cl | H |
| Z1.377 | H | H | CH₃ | F | 4-p-Cl-phenyl | 2-Cl | H |
| Z1.378 | H | H | CH₂CH₃ | F | 4-p-Cl-phenyl | 2-Cl | H |
| Z1.379 | H | H | (CH₂)₂CH₃ | F | 4-p-Cl-phenyl | 2-Cl | H |
| Z1.380 | H | H | CH(CH₃)₂ | F | 4-p-Cl-phenyl | 2-Cl | H |
| Z1.381 | H | H | F | F | 4-p-Cl-phenyl | 2-Cl | H |
| Z1.382 | CH₃ | H | CH₃ | F | 4-p-Cl-phenyl | 2-Cl | H |
| Z1.383 | CH₃ | H | CH₂CH₃ | F | 4-p-Cl-phenyl | 2-Cl | H |
| Z1.384 | CH₃ | H | (CH₂)₂CH₃ | F | 4-p-Cl-phenyl | 2-Cl | H |
| Z1.385 | CH₃ | H | CH(CH₃)₂ | F | 4-p-Cl-phenyl | 2-Cl | H |
| Z1.386 | CH₃ | H | F | F | 4-p-Cl-phenyl | 2-Cl | H |
| Z1.387 | (CH₂)₂ | | H | H | 4-p-Cl-phenyl | 2-Cl | H |
| Z1.388 | (CH₂)₂ | | CH₃ | H | 4-p-Cl-phenyl | 2-Cl | H |
| Z1.389 | (CH₂)₂ | | (CH₂)₂CH₃ | H | 4-p-Cl-phenyl | 2-Cl | H |
| Z1.390 | (CH₂)₂ | | CH(CH₃)₂ | H | 4-p-Cl-phenyl | 2-Cl | H |
| Z1.391 | H | H | H | H | 2-Cl | 4-Cl | 6-Cl |
| Z1.392 | CH₃ | H | H | H | 2-Cl | 4-Cl | 6-Cl |
| Z1.393 | CH₂CH₃ | H | H | H | 2-Cl | 4-Cl | 6-Cl |
| Z1.394 | (CH₂)₂CH₃ | H | H | H | 2-Cl | 4-Cl | 6-Cl |
| Z1.395 | CH(CH₃)₂ | H | H | H | 2-Cl | 4-Cl | 6-Cl |
| Z1.396 | F | H | H | H | 2-Cl | 4-Cl | 6-Cl |
| Z1.397 | H | H | CH₃ | H | 2-Cl | 4-Cl | 6-Cl |
| Z1.398 | H | H | CH₂CH₃ | H | 2-Cl | 4-Cl | 6-Cl |
| Z1.399 | CH₃ | CH₃ | H | H | 2-Cl | 4-Cl | 6-Cl |
| Z1.400 | CH₂CH₃ | CH₃ | H | H | 2-Cl | 4-Cl | 6-Cl |
| Z1.401 | F | CH₃ | H | H | 2-Cl | 4-Cl | 6-Cl |
| Z1.402 | CH₃ | H | CH₃ | H | 2-Cl | 4-Cl | 6-Cl |
| Z1.403 | CH₂CH₃ | H | CH₃ | H | 2-Cl | 4-Cl | 6-Cl |
| Z1.404 | CH₃ | H | F | H | 2-Cl | 4-Cl | 6-Cl |
| Z1.405 | CH₂CH₃ | H | F | H | 2-Cl | 4-Cl | 6-Cl |
| Z1.406 | H | H | CH₃ | CH₃ | 2-Cl | 4-Cl | 6-Cl |
| Z1.407 | H | H | CH₂CH₃ | CH₃ | 2-Cl | 4-Cl | 6-Cl |
| Z1.408 | H | H | CH₃ | F | 2-Cl | 4-Cl | 6-Cl |
| Z1.409 | H | H | CH₂CH₃ | F | 2-Cl | 4-Cl | 6-Cl |
| Z1.410 | H | H | F | F | 2-Cl | 4-Cl | 6-Cl |
| Z1.411 | CH₃ | H | CH₃ | F | 2-Cl | 4-Cl | 6-Cl |
| Z1.412 | CH₃ | H | CH₂CH₃ | F | 2-Cl | 4-Cl | 6-Cl |
| Z1.413 | CH₃ | H | F | F | 2-Cl | 4-Cl | 6-Cl |
| Z1.414 | (CH₂)₂ | | H | H | 2-Cl | 4-Cl | 6-Cl |
| Z1.415 | H | H | H | H | 2-F | 4-F | 6-F |
| Z1.416 | CH₃ | H | H | H | 2-F | 4-F | 6-F |
| Z1.417 | CH₂CH₃ | H | H | H | 2-F | 4-F | 6-F |
| Z1.418 | (CH₂)₂CH₃ | H | H | H | 2-F | 4-F | 6-F |
| Z1.419 | CH(CH₃)₂ | H | H | H | 2-F | 4-F | 6-F |
| Z1.420 | F | H | H | H | 2-F | 4-F | 6-F |
| Z1.421 | H | H | CH₃ | H | 2-F | 4-F | 6-F |
| Z1.422 | H | H | CH₂CH₃ | H | 2-F | 4-F | 6-F |
| Z1.423 | CH₃ | CH₃ | H | H | 2-F | 4-F | 6-F |
| Z1.424 | CH₂CH₃ | CH₃ | H | H | 2-F | 4-F | 6-F |
| Z1.425 | F | CH₃ | H | H | 2-F | 4-F | 6-F |
| Z1.426 | CH₃ | H | CH₃ | H | 2-F | 4-F | 6-F |
| Z1.427 | CH₂CH₃ | H | CH₃ | H | 2-F | 4-F | 6-F |
| Z1.428 | CH₃ | H | F | H | 2-F | 4-F | 6-F |
| Z1.429 | CH₂CH₃ | H | F | H | 2-F | 4-F | 6-F |
| Z1.430 | H | H | CH₃ | CH₃ | 2-F | 4-F | 6-F |
| Z1.431 | H | H | CH₂CH₃ | CH₃ | 2-F | 4-F | 6-F |
| Z1.432 | H | H | CH₃ | F | 2-F | 4-F | 6-F |
| Z1.433 | H | H | CH₂CH₃ | F | 2-F | 4-F | 6-F |
| Z1.434 | H | H | F | F | 2-F | 4-F | 6-F |
| Z1.435 | CH₃ | H | CH₃ | F | 2-F | 4-F | 6-F |
| Z1.436 | CH₃ | H | CH₂CH₃ | F | 2-F | 4-F | 6-F |
| Z1.437 | CH₃ | H | F | F | 2-F | 4-F | 6-F |
| Z1.438 | (CH₂)₂ | | H | H | 2-F | 4-F | 6-F |
| Z1.439 | CF₃ | H | H | H | 2-Cl | 4-Cl | H |
| Z1.440 | CF₂H | H | H | H | 2-Cl | 4-Cl | H |
| Z1.441 | CH₃ | H | H | H | 2-Cl | H | 6-F |
| Z1.442 | CH₃ | H | H | H | 2-Cl | H | 6-Cl |
| Z1.443 | CH₃ | H | H | H | 2-Cl | H | 6-CH₃ |
| Z1.444 | CH₂F | H | H | H | 2-Cl | 4-Cl | H |
| Z1.445 | CF₃ | H | H | H | 2-Cl | 4-Cl | 6-Cl |
| Z1.446 | H | H | H | H | 4-OH | H | H |
| Z1.447 | H | H | H | H | 4-O-(4′-CF₃-phenyl) | H | H |
| Z1.448 | H | H | H | H | 4-O-(3-Cl,5-CF₃-2- | H | H |
| | | | | | pyridinyl) | | |
| Z1.449 | CH₃ | H | H | H | 4-O-(3-Cl,5-CF₃-2- | 2-Cl | 6-Cl |
| | | | | | pyridinyl) | | |
| Z1.450 | CH₃ | H | H | H | 4-O-(4′-CF₃-phenyl) | 2-Cl | 6-Cl |
| Z1.451 | CH₃ | H | H | H | 2-Cl | 4-Br | H |
| Z1.452 | CH₃ | H | F | H | 2-Cl | 4-Br | H |
| Z1.453 | CH₃ | H | CH₃ | H | 2-Cl | 4-Br | H |
| Z1.454 | CF₂H | H | H | H | 2-Cl | 4-Cl | 6-Cl |
| Z1.455 | CH₂F | H | H | H | 2-Cl | 4-Cl | 6-Cl |
| Z1.456 | CH₃ | H | H | H | 2-Cl | 4-Br | 6-Cl |
| Z1.457 | CH₃ | H | F | H | 2-Cl | 4-Br | 6-Cl |
| Z1.458 | CH₃ | H | CH₃ | H | 2-Cl | 4-Br | 6-Cl |
| Z1.459 | CF₃ | H | H | H | 2-Br | 4-Br | 6-Cl |
| Z1.460 | CF₃ | H | H | H | 2-Br | 4-Br | 6-Cl |
| Z1.461 | CH₃ | H | H | H | 4-(2′-Cl-phenyl) | 2-Cl | H |
| Z1.462 | CH₃ | H | H | H | 4-(4′-Cl-phenyl) | 2-Cl | H |
| Z1.463 | CH₃ | H | H | H | 4-(4′-CF₃-phenyl) | 2-Cl | H |
| Z1.464 | CH₃ | H | H | H | 4-(4′-OCH₃-phenyl) | 2-Cl | H |
| Z1.465 | CH₃ | H | H | H | 4-(3′,4′-Cl₂-phenyl) | 2-Cl | H |
| Z1.466 | CH₃ | H | H | H | 4-(2′-Cl-phenyl) | 2-Cl | 6-Cl |
| Z1.467 | CH₃ | H | H | H | 4-(4′-CF₃-phenyl) | 2-Cl | 6-Cl |
| Z1.468 | CH₃ | H | H | H | 4-(4′-OCH₃-phenyl) | 2-Cl | 6-Cl |
| Z1.469 | CH₃ | H | H | H | 4-(3′,4′-Cl₂-phenyl) | 2-Cl | 6-Cl |
| Z1.470 | CH₃ | H | H | H | 4-C≡CSi(CH₃)₃ | 2-Cl | H |
| Z1.471 | CH₃ | H | H | H | 4-C≡CH | 2-Cl | H |
| Z1.472 | CH₃ | H | H | H | 4-C≡CC(CH₃)₃ | 2-Cl | H |
| Z1.473 | CH₃ | H | H | H | 4-C≡CCH(CH₂)₂ | 2-Cl | H |
| Z1.474 | CH₃ | H | H | H | 4-C≡C-(4-Cl-phenyl) | 2-Cl | H |
| Z1.475 | CH₃ | H | H | H | 4-C≡CC(CH₃)₃ | 2-Cl | 6-Cl |
| Z1.476 | CH₃ | H | H | H | 4-C≡CCH(CH₂)₂ | 2-Cl | 6-Cl |
| Z1.477 | CH₃ | H | H | H | 4-C≡C-(4-Cl-phenyl) | 2-Cl | 6-Cl |
| Z1.478 | CH₃ | H | H | H | 4-CHO | 2-Cl | H |
| Z1.479 | CH₃ | H | H | H | 4-CH═NOCH₃ | 2-Cl | H |
| Z1.480 | CH₃ | H | H | H | 4-COCH₃ | 2-Cl | H |
| Z1.481 | CH₃ | H | H | H | 4-C(CH₃)═NOCH₃ | 2-Cl | H |
| Z1.482 | CH₃ | H | H | H | 4-CH₃ | 2-CH₃ | 6-CH₃ |

### Table 14: Characterising Data

Table 14 shows selected melting point and selected NMR data for compounds of Tables 1 to 13. CDCl₃ was used as the solvent for NMR measurements, unless otherwise stated. If a mixture of solvents was present, this is indicated as, for example: CDCl₃/d₆-DMSO). No attempt is made to list all characterising data in all cases.

In Table 14 and throughout the description that follows, temperatures are given in degrees Celsius; “NMR” means nuclear magnetic resonance spectrum; MS stands for mass spectrum; “%” is percent by weight, unless corresponding concentrations are indicated in other units. The following abbreviations are used throughout this description:
m.p.=melting point
S=singlet
d=doublet
t=triplet
b.p.=boiling point.
br=broad
dd=doublet of doublets
q=quartet

| Compound | | | |
|---|---|---|---|
| No. | 1H-NMR data: ppm (multiplicity/number of Hs) | MS [M + H]⁺ | m.p. (° C.) |
| 1.001 | 2.86(t, 2H), 3.64(q, 2H), 3.84(s, 3H), 6.40(t, 1H), | 314/316 | liquid |
| | 6.79(t, 1H), 7.14(d, 2H), 7.23(d, 2H), | | |
| | 7.85(s, 1H). | | |
| 1.002 | 1.18(d, 3H), 2.75(dd, 1H), 2.87(dd, 1H), | 328/330 | 105-112 |
| | 3.91(s, 3H), 4.37(m, 1H), 6.22(t, 1H), | | |
| | 6.78(t, 1H), 7.13(d, 2H), 7.24(d, 2H), | | |
| | 7.90(s, 1H). | | |
| 1.036 | 1.11(m, 1H), 1.44(q, 3H), 2.35(q, 1H), | 326/328 | 127 |
| | 3.26(m, 1H), 3.73(s, 3H), 6.08(s, 1H), | | |
| | 6.62(t, 1H), 7.15(m, 2H), 7.21(d, 2H), | | |
| | 7.70(s, 1H). | | |
| 1.196 | 3.00(t, 2H), 3.66(q, 2H), 3.92(s, 3H), 6.36(t, 1H), | 348/350/352 | liquid |
| | 6.79(t, 1H), 7.18(m, 2H), 7.39(d, 1H), | | |
| | 7.88(s, 1H). | | |
| 1.197 | 1.25(d, 3H), 2.98(m, 2H), 3.91(s, 3H), | 362/364/366 | 157 |
| | 4.45(m, 1H), 6.21(t, 1H), 6.77(t, 1H), | | |
| | 7.16(m, 2H), 7.36(d, 1H), 7.85(s, 1H). | | |
| 1.198 | 1.004(t, 3H), 1.68(m, 2H), 2.90(dd, 1H), | 376/378/380 | 138-140 |
| | 2.99(dd, 1H), 3.90(s, 3H), 4.35(m, 1H), | | |
| | 6.15(s, 1H), 6.64(t, 1H), 7.14(dd, 1H), | | |
| | 7.18(d, 1H), 7.35(d, 1H), 7.84(s, 1H) | | |
| 1.199 | 0.9(t, 3H), 1.4(m, 4H), 2.86(dd, 1H), 2.88(m, 1H), | 390/392/394 | 108-109 |
| | 3.9(s, 3H), 4.41(m, 1H), 6.14(sbr, 1H), 6.78(t, 1H), | | |
| | 7.1(dd, 1H), 7.23(d, 1H)7.35(d, 1H), 7.83(s1H) | | |
| 1.202 | 1.28(d, 3H), 3.56-3.64(m, 2H + 1H), 3.83(s, 3H), | 362/364/366 | liquid |
| | 6.37(t, 1H), 6.89(t, 1H), 7.25(m, 2H), | | |
| | 7.38(d, 1H), 7.83(s, 1H). | | |
| 1.204 | 0.88(t, 3H), 1.18-1.31(m, 2H), 1.56- | 390/392/394 | liquid |
| | 1.75(m, 2H), 3.48(m, 2H), 3.75(m, 1H), | | |
| | 3.87(s, 3H), 6.18(t, 1H), 6.78(t, 1H), | | |
| | 7.23(m, 2H), 7.38(d, 1H), 7.83(s, 1H). | | |
| 1.205 | 0.80(d, 3H), 1.08(d, 3H), 1.92(m, 1H), | 390/392/394 | liquid |
| | 3.28(m, 1H), 3.54(m, 1H), 3.87(s, 3H), | | |
| | 3.94(m, 1H), 6.03(t, 1H), 6.65(t, 1H), | | |
| | 7.23(m, 2H), 7.39(d, 1H), 7.81(s, 1H). | | |
| 1.206 | 3.62-3.75(m, 1H), 3.92(s, 3H), 4.03- | 366/368/370 | liquid |
| | 4.15(m, 1H), 5.87 + 5.99(dd, 1H), 6.67(t, 1H), | | |
| | 6.82(t, 1H), 7.29(dd, 1H), 7.41(d, 1H), | | |
| | 7.43(d, 1H), 7.91(s, 1H). | | |
| 1.207 | 1.37(s, 6H), 3.14(s, 2H), 3.91(s, 3H), | 376/378/380 | 94-96 |
| | 5.97(s, 1H), 6.76(t, 1H), 6.98(dxd, 1H), | | |
| | 7.25(d, 1H), 7.30(d, 1H), 7.85(s, 1H). | | |
| 1.216 | 1.43(d, 3H, CH3), 3.87(s, 3H, NCH3), 4.69- | 380/382/384 | resin |
| | 4.80(m, 1H, CH), 5.73 and 5.84(d, 1H, CH), | | |
| | 6.51(t, 1H, NH), 6.79(t, 1H, CHF2), | | |
| | 7.19(d, 1H, Ar—H), 7.35-7.37(m, 2H, Ar—H), | | |
| | 7.79(s, 1H, Pyrazol-H). | | |
| 1.221 | 1.82 + 1.87(2s, 3H), 3.90(s, 3H), 4.15(dd, 1H), | 380/382/384 | solid |
| | 4.22(dd, 1H), 6.52(t, 1H), 6.73(t, 1H), | | |
| | 7.25(m, 1H), 7.39(d, 1H), 7.52(d, 1H), | | |
| | 7.86(s, 1H). | | |
| 1.231 | 0.88-0.99 (m, 4H, 2xCH2), 3.18(s, 3H, CH3), | 374/376/378 | 165-168 |
| | 3.86(s, 3H, NCH3), 6.45(t, 1H, NH), | | |
| | 6.76(t, 1H, CHF2), 7.13(m, 1H, Ar—H), | | |
| | 7.22(d, 1H, Ar—H), 7.40(d, 1H, Ar—H), | | |
| | 7.86(s, 1H, Pyrazol-H). | | |
| 1.235 | 2.86(t, 2H), 3.61(q, 2H), 3.93(s, 3H), 6.39(t, 1H), | 348/350/352 | liquid |
| | 6.77(t, 1H), 7.08(dd, 1H), 7.32(dd, 1H), | | |
| | 7.39(d, 1H), 7.89(s, 1H). | | |
| 1.275 | 1.21(d, 3H), 2.84-2.86(dd*dd, 2H), 3.90(s, 3H), | 346 | 146-148 |
| | 4.36-4.42(m, 1H), 6.18(s, 1H), 6.78(t, 1H), | | |
| | 7.05(d, 1H), 7.15(d, 1H), 7.85(s, 1H) | | |
| 1.392 | 1.24(d, 3H), 2.99(dd, 1H), 3.13(dd, 1H), | 396/398/400 | 140-143 |
| | 3.88(s, 1H), 4.56(m, 1H), 6.18(s, 1H), | | |
| | 6.78(t, 1H), 7.19(s, 2H), 7.70(s, 1H) | | |
| 1.416 | 1.24(d, 3H), 2.87(d, 2H), 3.89(s, 3H), | 348 | solid |
| | 4.41(m, 1H), 6.27(s, 1H), 6.70(m, 2H), | | |
| | 7.82(s, 1H). | | |
| 1.441 | 1.26(d, 3H), 3.01(m, 2H), 3.87(s, 3H), | 346/348/350 | resin |
| | 4.52(m, 1H), 6.39(d, 1H), 6.94(m, 2H), 7.10- | | |
| | 7.16(m, 2H), 7.77(s, 1H). | | |
| 1.442 | 1.25(d, 3H), 3.05(dd, 1H), 3.15(dd, 1H), | 362/364/366 | gum |
| | 3.84(s.3H), 4.58(m, 1H), 6.16(s, 1H), 6.8(t, 1H), | | |
| | 7.01(t, 1H), 7.19(d, 2H), 7.67(s, 1H) | | |
| 1.443 | 1.2(d, 3H), 2.45(s, 3H), 2.94(dd, 1H), | 342/344 | 93-95 |
| | 3.15(dd, 1H), 3.96(s, 3H), 4.45(m, 1H), | | |
| | 6.3(s, 1H), 6.9(t, 1H), 7.04(m, 2H), 7.19(d, 1H), | | |
| | 7.79(s, 1H) | | |
| 1.446 | 2.80(t, 2H), 3.62(q, 2H), 3.86(s, 3H), | 296 | resin |
| | 6.46(m, 1H), 6.78(d, 2H), 6.79(t, 1H), | | |
| | 7.04(d, 2H), 7.85(s, 1H). | | |
| 1.448 | 2.94(t, 2H), 3.69(q, 2H), 3.91(s, 3H), 6.44(s, 1H), | 475/477 | resin |
| | 6.94(t, 1H), 7.10(d, 1H), 7.30(d, 2H), | | |
| | 7.89(s, 1H), 7.98(d, 1H), 8.01(d, 1H), | | |
| | 8.58(s, 1H). | | |
| 1.451 | 1.2(d, 3H), 2.95(m, 2H), 3.85(s, 3H), | 406/408/410 | 183-184 |
| | 4.43(m, 1H), 6.13(s, 1H), 6.72(t, 1H), | | |
| | 7.08(d, 1H), 7.3(d, 1H), 7.44(s, 1H), 7.78(s, 1H). | | |
| 1.461 | 1.24(d, 3H), 2.91(dd, 1H), 3.01(dd, 1H), | 438/437/442 | 120-121 |
| | 3.84(s, 3H), 4.45(m, 1H), 6.18(s, 1H), | | |
| | 6.89(t, 1H), 7.25(m, 5H), 7.27(m, 2H), | | |
| | 7.39(d, 1H), 7.78(s, 1H) | | |
| 1.462 | 1.22(d, 3H), 2.96(m, 2H), 3.83(s, 3H), | 438/440/442 | 96-98 |
| | 4.45(m, 1H), 6.15(s, 1H), 6.75(t, 1H,), 7.05- | | |
| | 7.44(m, 6H), 7.45(d, 1H), 7.8(s, 1H). | | |
| 1.463 | 1.21(d, 3H), 2.95(dd, 1H), 3.01(dd, 1H), | 472/474/476 | 128-130 |
| | 3.83(s, 3H), 4.46(m, 1H), 6.20(t, 1H), | | |
| | 6.74(t, 1H), 7.34(m, 2H), 7.58(m, 5H) 7.8(s, 1H) | | |
| 1.464 | 1.21(d, 3H), 2.95(dd, 1H), 3.01(dd, 1H), | 433.93 | resin |
| | 3.77(s, 3H), 3.83(s, 3H), 4.43(m, 1H), | | |
| | 6.2(s, 1H), 6.87(t, 1H), 6.90(d, 2H), 7.19- | | |
| | 7.42(m, 4H), 7.45(d, 1H), 7.8(s, 1H) | | |
| 1.465 | 1.21(d, 3H), 2.90-3.02(dd*dd, 2H), 3.89(s, 3H), | 472/474/476 | 126-128 |
| | 4.46(m, 1H), 6.2(s, 1H), 6.75(t, 1H), | | |
| | 7.32(m, 2H), 7.41-7.50(m, 3H), 7.55(d, 1H), | | |
| | 7.78(s, 1H) | | |
| 1.482 | 1.10(d, 3H), 2.17(d, 3H), 2.30(s, 6H), | 337 | 120-121 |
| | 2.65(dd, 1H), 3.02(dd, 1H), 3.85(s, 3H), 4.24- | | |
| | 4.33(m, 1H), 6.25(s, 1H), 6.65 (t, 1H), | | |
| | 6.77(d, 2H), 6.85(s, 1H) | | |
| 2.001 | 2.87(t, 2H), 3.66(q, 2H), 3.93(s, 3H), 6.06(t, 1H), | 332/334 | liquid |
| | 7.15(d, 2H), 7.28(d, 2H), 7.87(s, 1H). | | |
| 2.157 | 2.94(t, 2H), 3.70(q, 2H), 3.94(s, 3H), 6.05(t, 1H), | 408/410 | 158-162 |
| | 7.30(d, 2H), 7.40(d, 2H), 7.50(m, 4H), | | |
| | 7.90(s, 1H). | | |
| 2.196 | 3.01(t, 2H), 3.66(q, 2H), 3.95(s, 3H), 6.05(t, 1H), | 366/368/370 | liquid |
| | 7.19(m, 2H), 7.39(d, 1H), 7.89(s, 1H). | | |
| 2.197 | 1.25(d, 3H), 2.96(m, 2H), 3.94(s, 3H), | 380/382/384 | 152-154 |
| | 4.46(m, 1H), 5.89(d, 1H), 7.18(dxd, 2H), | | |
| | 7.37(s, 1H), 7.85(s, 1H). | | |
| 2.198 | 0.935(t, 3H), 1.47(m, 1H), 1.65(m, 1H), | 394/396 | 119-120 |
| | 2.83(dd, 1H), 2.93(dd, 1H), 3.87(s, 3H), | | |
| | 4.26(m, 1H), 5.76(d, 1H), 7.11(m, 2H), | | |
| | 7.28(d, 1H), 7.77(s.1H) | | |
| 2.199 | 0.87(t, 3H), 1.41-1.50(m, 4H), 2.82(dd, 1H), | 408/406 | 104-105 |
| | 2.95(dd, 1H), 3.86(s, 3H), 4.35(m, 1H), | | |
| | 5.74(d, 1H), 7.08-7.10(m, 2H), 7.28(d, 1H), | | |
| | 7.76(s, 1H) | | |
| 2.206 | 3.60-3.71(m, 1H), 3.92(s, 3H), 4.03- | 384/386/388 | liquid |
| | 4.16(m, 1H), 5.87 + 5.98(dd, 1H), 6.37(t, 1H), | | |
| | 7.32(dd, 1H), 7.41(m, 2H), 7.93(s, 1H). | | |
| 2.235 | 2.85(t, 2H), 3.61(q, 2H), 3.94(s, 3H), 6.09(t, 1H), | 366/368/370 | solid |
| | 7.06(dd, 1H), 7.31(dd, 1H), | | |
| | 7.38(d, 1H), 7.88(s, 1H). | | |
| 3.197 | 1.28(d, 3H), 2.71(s, 3H), 3.96(ddxdd, 2H), | 397/399/401 | 140-141 |
| | 4.44(m, 1H), 6.04(d, 1H), 7.19(s, 3H), | | |
| | 7.38(s, 1H). | | |
| 3.198 | 0.985(t, 3H), 1.58(m, 1H), 1.73(m, 1H), | 411 | 140-143 |
| | 2.71(s, 3H), 2.90(dd, 1H), 2.99(dd, 1H), | | |
| | 4.30(m, 1H), 5.92(md, 1H), 7.18(d, 2H), | | |
| | 7.37(s, 1H) | | |
| 3.199 | 0.94(t, 3H), 1.45(m, 4H), 2.72(s, 3H), | 425/423 | 149-150 |
| | 2.92(dd, 1H), 3.01(dd, 1H), 4.37(m, 1H), | | |
| | 5.88(sbr, 1H), 7.18(d, 2H), 7.37(s, 1H) | | |
| 3.200 | 1.03(d, 6H), 1.96(m, 1H), 2.70(s, 3H), 2.81- | 425/427/429 | 185-186 |
| | 2.84(dd, 1H), 2.98-3.03(dd, 1H), 4.31(m, 1H), | | |
| | 5.95(d, 1H), 7.17(s, 2H), 7.35(s, 1H) | | |
| 3.212 | 1.28(dd, 3H), 1.31(dd, 3H), 2.73(s, 3H), | 412/414/416 | semisolid |
| | 3.63(m, 1H), 5.32(m, 1H), 7.23(d, 1H), | | |
| | 7.24(s, 1H), 7.38(d, 1H) | | |
| 5.197 | 1.22(d, 3H), 2.95(ddxdd, 2H), 3.63(s, 3H), | 379/381/383 | 115-116 |
| | 4.44(m, 1H), 6.01(d, 1H), 6.90(s, 1H), | | |
| | 7.13(s, 1H), 7.14(d, 1H), 7.21(s, 1H), | | |
| | 7.33(d, 1H). | | |
| 5.198 | 0.99(t, 3H), 1.55(m, 1H), 1.67(m, 1H), | 393/395/396.9 | 160-162 |
| | 2.89(dd, 1H), 2.97(dd, 1H), 3.66(s, 3H), | | |
| | 4.33(m, 1H), 5.83(d, 1H), 6.9(s, 1H), | | |
| | 7.14(m, 2H), 7.17(d, 1H), 7.34(s, 1H) | | |
| 5.199 | 0.85(t, 3H), 1.25-1.49(m, 4H), 2.79(dd, 1H), | 407/409 | 143-144 |
| | 2.95(dd, 1H), 4.35(m, 1H), 5.75(d, 1H), | | |
| | 6.85(s, 1H), 7.07(d, 1H), 7.10(dd, 1H), | | |
| | 7.15(d, 1H), 7.28(d, 1H) | | |
| Z1.197 | | 204/206/208 | liquid |
| free base | | | |
| Z1.206 | | 208/210/212 | solid |
| HCl-salt | | | |
| Z1.216 | | 222/224/226 | solid |
| HCl-salt | | | |
| Z1.221 | (DMSO): δ 8.44(sbr, 2H), 7.72(d, 1H), | 222/224/226 | 152-155 |
| HCl-salt | 7.65(d, 1H), 7.53(dd, 1H), 3.55(m, 2H), | | |
| | 1.85(d, 3H, J = 28 Hz) | | |
| Z1.231 | | 216/218/220 | liquid |
| free base | | | |
| Z1.451 | 1.25(d, 3H ), 2.95(dd, 2H), 3.2(m, 1H), 4.42(m, | 248/250/252 | liquid |
| free base | 2H), 7.3(d, 1H), 7.5(dd, 1H), 7.7(d, 1H). | | |

### Formulation Examples for Compounds of Formula I

The Formulation Examples which follow serve to illustrate the invention and relate to the manufacture of compositions comprising compounds of formula I, such as the compounds of tables 1 to 12. The same Formulation Examples can be used to make compositions comprising compounds of formula IA, such as the compounds described in table 15.

### Example F-1.1 to F-1.3

### Emulsifiable Concentrates

| Components | F-1.1 | F-1.2 | F-1.3 |
|---|---|---|---|
| compound of Tables 1 to 12 | 25% | 40% | 50% |
| calcium dodecylbenzenesulfonate | 5% | 8% | 6% |
| castor oil polyethylene glycol ether | 5% | — | — |
| (36 mol ethylenoxy units) | | | |
| tributylphenolpolyethylene glycol ether | — | 12% | 4% |
| (30 mol ethylenoxy units) | | | |
| cyclohexanone | — | 15% | 20% |
| xylene mixture | 65% | 25% | 20% |

Emulsions of any desired concentration can be prepared by diluting such concentrates with water.

### Example F-2

### Emulsifiable Concentrate

| Components | F-2 |
|---|---|
| compound of Tables 1 to 12 | 10% |
| octylphenolpolyethylene glycol ether | 3% |
| (4 to 5 mol ethylenoxy units) | |
| calcium dodecylbenzenesulfonate | 3% |
| castor oil polyglycol ether | 4% |
| (36 mol ethylenoxy units) | |
| cyclohexanone | 30% |
| xylene mixture | 50% |

Emulsions of any desired concentration can be prepared by diluting such concentrates with water.

### Examples F-3.1 to F-3.4

### Solutions

| Components | F-3.1 | F-3.2 | F-3.3 | F-3.4 |
|---|---|---|---|---|
| compound of Tables 1 to 12 | 80% | 10% | 5% | 95% |
| propylene glycol monomethyl ether | 20% | — | — | — |
| polyethylene glycol (relative molecular | — | 70% | — | — |
| mass: 400 atomic mass units) | | | | |
| N-methylpyrrolid-2-one | — | 20% | — | — |
| epoxidised coconut oil | — | — | 1% | 5% |
| benzin (boiling range: 160-190°) | — | — | 94% | — |

The solutions are suitable for use in the form of microdrops.

### Examples F-4.1 to F-4.4

### Granulates

| Components | F-4.1 | F-4.2 | F-4.3 | F-4.4 |
|---|---|---|---|---|
| compound of Tables 1 to 12 | 5% | 10% | 8% | 21% |
| kaolin | 94% | — | 79% | 54% |
| highly dispersed silicic acid | 1% | — | 13% | 7% |
| attapulgite | — | 90% | — | 18% |

The novel compound is dissolved in dichloromethane, the solution is sprayed onto the carrier and the solvent is then removed by distillation under vacuum.

### Examples F-5.1 and F-5.2

### Dusts

| Components | F-5.1 | F-5.2 |
|---|---|---|
| compound of Tables 1 to 12 | 2% | 5% |
| highly dispersed silicic acid | 1% | 5% |
| talcum | 97% | — |
| kaolin | — | 90% |

Ready for use dusts are obtained by intimately mixing all components.

### Examples F-6.1 to F-6.3

### Wettable Powders

| Components | F-6.1 | F-6.2 | F-6.3 |
|---|---|---|---|
| compound of Tables 1 to 12 | 25% | 50% | 75% |
| sodium lignin sulfonate | 5% | 5% | — |
| sodium lauryl sulfate | 3% | — | 5% |
| sodium diisobutylnaphthalene sulfonate | — | 6% | 10% |
| octylphenolpolyethylene glycol ether | — | 2% | — |
| (7 to 8 mol ethylenoxy units) | | | |
| highly dispersed silicic acid | 5% | 10% | 10% |
| kaolin | 62% | 27% | — |

All components are mixed and the mixture is thoroughly ground in a suitable mill to give wettable powders which can be diluted with water to suspensions of any desired concentration.

### Example F7

### Flowable Concentrate for Seed Treatment

| | |
|---|---|
| compound of Tables 1 to 12 | 40% |
| propylene glycol | 5% |
| copolymer butanol PO/EO | 2% |
| tristyrenephenole with 10-20 moles EO | 2% |
| 1,2-benzisothiazolin-3-one (in the form of a 20% | 0.5% |
| solution in water) | |
| monoazo-pigment calcium salt | 5% |
| Silicone oil (in the form of a 75% emulsion in water) | 0.2% |
| Water | 45.3% |

The finely ground active ingredient is intimately mixed with the adjuvants, giving a suspension concentrate from which suspensions of any desired dilution can be obtained by dilution with water. Using such dilutions, living plants as well as plant propagation material can be treated and protected against infestation by microorganisms, by spraying, pouring or immersion.

### BIOLOGICAL EXAMPLES

### Fungicidal Actions

### Example B-1: Action Against Podosphaera leucotricha 1 Apple (Powdery Mildew on Apple)

5 week old apple seedlings cv. McIntosh are treated with the formulated test compound (0.02% active ingredient) in a spray chamber. One day after application apple plants are inoculated by shaking plants infected with apple powdery mildew above the test plants. After an incubation period of 12 days at 22° C. and 60% r.h. under a light regime of 14/10 hours (light/dark) the disease incidence is assessed. Compounds 1.001, 1.002, 1.036, 1.196, 1.197, 1.198, 1.199, 1.202, 1.204, 1.205, 1.206, 1.207, 1.216, 1.221, 1.231, 1.235, 1.275, 1.392, 1.416, 1.441, 1.442, 1.443, 1.446, 1.448, 1.451, 1.461, 1.462, 1.463, 1.464, 1.465, 1.482, 2.001, 2.157, 2.196, 2.197, 2.198, 2.199, 2.206, 2.235, 3.197, 3.198, 3.199, 3.200, 3.212, 5.197, 5.198 and 5.199 show good activity in this test (<20% infestation).

### Example B-2: Action Against Venturia inaequalis/Apple (Scab on Apple)

4 week old apple seedlings cv. McIntosh are treated with the formulated test compound (0.02% active ingredient) in a spray chamber. One day after application apple plants are inoculated by spraying a spore suspension (4×10⁵ conidia/ml) on the test plants. After an incubation period of 4 days at 21° C. and 95% r.h. the plants are placed for 4 days at 21° C. and 60% r.h. in a greenhouse. After another 4 day incubation period at 21° C. and 95% r.h. the disease incidence is assessed. Compounds 1.001, 1.002, 1.036, 1.196, 1.197, 1.198, 1.199, 1.202, 1.204, 1.205, 1.206, 1.207, 1.216, 1.221, 1.231, 1.235, 1.275, 1.392, 1.416, 1.441, 1.442, 1.443, 1.446, 1.448, 1.451, 1.461, 1.462, 1.463, 1.464, 1.465, 1.482, 2.001, 2.157, 2.196, 2.197, 2.198, 2.199, 2.206, 2.235, 3.197, 3.198, 3.199, 3.200, 3.212, 5.197, 5.198 and 5.199 show good activity in this test (<20% infestation).

### Example B-3: Action Against Erysiphe graminis/Barley (Powdery Mildew on Barley)

1 week old barley plants cv. Express are treated with the formulated test compound (0.02% active ingredient) in a spray chamber. One day after application barley plants are inoculated by shaking powdery mildew infected plants above the test plants. After an incubation period of 6 days at 20° C./18° C. (day/night) and 60% r. h. in a greenhouse the disease incidence is assessed. Compounds 1.001, 1.002, 1.036, 1.196, 1.197, 1.198, 1.199, 1.202, 1.204, 1.205, 1.206, 1.207, 1.216, 1.221, 1.231, 1.235, 1.275, 1.392, 1.416, 1.441, 1.442, 1.443, 1.446, 1.448, 1.451, 1.461, 1.462, 1.463, 1.464, 1.465, 1.482, 2.001, 2.157, 2.196, 2.197, 2.198, 2.199, 2.206, 2.235, 3.197, 3.198, 3.199, 3.200, 3.212, 5.197, 5.198 and 5.199 show good activity in this test (<20% infestation).

### Example B-4: Action Against Botrytis cinerea/Apple (Botrytis on Apple Fruits)

In an apple fruit cv. Golden Delicious 3 holes are drilled and each filled with 30 μl droplets of the formulated test compound (0.02% active ingredient). Two hours after application 50 μl of a spore suspension of B. cinerea (4×10⁵ conidia/ml) are pipetted on the application sites. After an incubation period of 7 days at 22° C. in a growth chamber the disease incidence is assessed. Compounds 1.001, 1.002, 1.036, 1.196, 1.197, 1.198, 1.199, 1.202, 1.204, 1.205, 1.206, 1.207, 1.216, 1.221, 1.231, 1.235, 1.275, 1.392, 1.416, 1.441, 1.442, 1.443, 1.446, 1.448, 1.451, 1.461, 1.462, 1.463, 1.464, 1.465, 1.482, 2.001, 2.157, 2.196, 2.197, 2.198, 2.199, 2.206, 2.235, 3.197, 3.198, 3.199, 3.200, 3.212, 5.197, 5.198 and 5.199 show good activity in this test (<20% infestation).

### Example B-5: Action Against Botrytis cinerea/Grape (Botrytis on Grapes)

5 week old grape seedlings cv. Gutedel are treated with the formulated test compound (0.02% active ingredient) in a spray chamber. Two days after application grape plants are inoculated by spraying a spore suspension (1×10⁵ conidia/ml) on the test plants. After an incubation period of 4 days at 21° C. and 95% r.h. in a greenhouse the disease incidence is assessed. Compounds 1.001, 1.002, 1.036, 1.196, 1.197, 1.198, 1.199, 1.202, 1.204, 1.205, 1.206, 1.207, 1.216, 1.221, 1.231, 1.235, 1.275, 1.392, 1.416, 1.441, 1.442, 1.443, 1.446, 1.448, 1.451, 1.461, 1.462, 1.463, 1.464, 1.465, 1.482, 2.001, 2.157, 2.196, 2.197, 2.198, 2.199, 2.206, 2.235, 3.197, 3.198, 3.199, 3.200, 3.212, 5.197, 5.198 and 5.199 show good activity in this test (<20% infestation).

### Example B-6: Action Against Botrytis cinerea/Tomato (Botrytis on Tomatoes)

4 week old tomato plants cv. Roter Gnom are treated with the formulated test compound (0.02% active ingredient) in a spray chamber. Two days after application tomato plants are inoculated by spraying a spore suspension (1×10⁵ conidia/ml) on the test plants. After an incubation period of 4 days at 20° C. and 95% r.h. in a growth chamber the disease incidence is assessed. Compounds 1.001, 1.002, 1.036, 1.196, 1.197, 1.198, 1.199, 1.202, 1.204, 1.205, 1.206, 1.207, 1.216, 1.221, 1.231, 1.235, 1.275, 1.392, 1.416, 1.441, 1.442, 1.443, 1.446, 1.448, 1.451, 1.461, 1.462, 1.463, 1.464, 1.465, 1.482, 2.001, 2.157, 2.196, 2.197, 2.198, 2.199, 2.206, 2.235, 3.197, 3.198, 3.199, 3.200, 3.212, 5.197, 5.198 and 5.199 show good activity in this test (<20% infestation).

### Example B-7: Action Against Pyrenophora teres/Barley (Net Blotch on Barley)

1 week old barley plants cv. Express are treated with the formulated test compound (0.02% active ingredient) in a spray chamber. Two days after application barley plants are inoculated by spraying a spore suspension (3×10⁴ conidia/ml) on the test plants. After an incubation period of 2 days at 20° C. and 95% r.h. plants are kept for 2 days at 20° C. and 60% r.h. in a greenhouse. The disease incidence is assessed 4 days after inoculation. Compounds 1.001, 1.002, 1.036, 1.196, 1.197, 1.198, 1.199, 1.202, 1.204, 1.205, 1.206, 1.207, 1.216, 1.221, 1.231, 1.235, 1.275, 1.392, 1.416, 1.441, 1.442, 1.443, 1.446, 1.448, 1.451, 1.461, 1.462, 1.463, 1.464, 1.465, 1.482, 2.001, 2.157, 2.196, 2.197, 2.198, 2.199, 2.206, 2.235, 3.197, 3.198, 3.199, 3.200, 3.212, 5.197, 5.198 and 5.199 show good activity in this test (<20% infestation).

### Example B-8: Action Against Septoria tritici/Wheat (Septoria Leaf Spot on Wheat)

2 week old wheat plants cv. Riband are treated with the formulated test compound (0.02% active ingredient) in a spray chamber. One day after application, wheat plants are inoculated by spraying a spore suspension (10×10⁵ conidia/ml) on the test plants. After an incubation period of 1 day at 23° C. and 95% r.h., the plants are kept for 16 days at 23° C. and 60% r.h. in a greenhouse. The disease incidence is assessed 18 days after inoculation. Compounds 1.001, 1.002, 1.036, 1.196, 1.197, 1.198, 1.199, 1.202, 1.204, 1.205, 1.206, 1.207, 1.216, 1.221, 1.231, 1.235, 1.275, 1.392, 1.416, 1.441, 1.442, 1.443, 1.446, 1.448, 1.451, 1.461, 1.462, 1.463, 1.464, 1.465, 1.482, 2.001, 2.157, 2.196, 2.197, 2.198, 2.199, 2.206, 2.235, 3.197, 3.198, 3.199, 3.200, 3.212, 5.197, 5.198 and 5.199 show good activity in this test (<20% infestation).

### Example B-9: Action Against Uncinula necator/Grape (Powdery Mildew on Grape)

5 week old grape seedlings cv. Gutedel are treated with the formulated test compound (0.02% active ingredient) in a spray chamber. One day after application, the grape plants are inoculated by shaking plants infected with grape powdery mildew above the test plants. After an incubation period of 7 days at 26° C. and 60% r.h. under a light regime of 14/10 hours (light/dark) the disease incidence is assessed. Compounds 1.001, 1.002, 1.036, 1.196, 1.197, 1.198, 1.199, 1.202, 1.204, 1.205, 1.206, 1.207, 1.216, 1.221, 1.231, 1.235, 1.275, 1.392, 1.416, 1.441, 1.442, 1.443, 1.446, 1.448, 1.451, 1.461, 1.462, 1.463, 1.464, 1.465, 1.482, 2.001, 2.157, 2.196, 2.197, 2.198, 2.199, 2.206, 2.235, 3.197, 3.198, 3.199, 3.200, 3.212, 5.197, 5.198 and 5.199 show good activity in this test (<20% infestation).

### Example B-10: Action Against Alternaria solani/Tomato (Early Blight on Tomatoes)

4 week old tomato plants cv. Roter Gnom are treated with the formulated test compound (0.02% active ingredient) in a spray chamber. Two days after application, the tomato plants are inoculated by spraying a spore suspension (2×10⁵ conidia/ml) on the test plants. After an incubation period of 3 days at 20° C. and 95% r.h. in a growth chamber the disease incidence is assessed. Compounds 1.001, 1.002, 1.036, 1.196, 1.197, 1.198, 1.199, 1.202, 1.204, 1.205, 1.206, 1.207, 1.216, 1.221, 1.231, 1.235, 1.275, 1.392, 1.416, 1.441, 1.442, 1.443, 1.446, 1.448, 1.451, 1.461, 1.462, 1.463, 1.464, 1.465, 1.482, 2.001, 2.157, 2.196, 2.197, 2.198, 2.199, 2.206, 2.235, 3.197, 3.198, 3.199, 3.200, 3.212, 5.197, 5.198 and 5.199 show good activity in this test (<20% infestation).

The present invention further relates to novel optically active ethyl amides having microbiocidal, in particular fungicidal, activity; to compositions which comprise these compounds; and to their use in agriculture or horticulture for controlling or preventing infestation of plants by phytopathogenic microorganisms, preferably fungi.

The present invention thus provides a compound of the formula IA

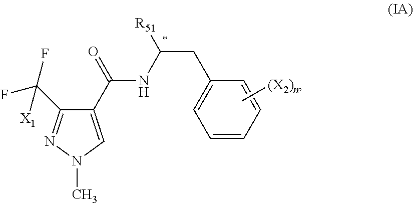

wherein R₅₁ is C₁-C₃alkyl, CF₃ or CF₂H; X₁ is hydrogen or fluoro; n is 2 or 3; each X₂ independently of each other stands for chloro, bromo, fluoro, CH₃ or CF₃; having an optical activity [α]_{D} of greater than 0° when dissolved in an achiral solvent.

The alkyl groups occurring in the definitions of the substituents can be straight-chain or branched and are, for example, methyl, ethyl, n-propyl or iso-propyl.

The compounds of formula IA have one chiral carbon atom, which is highlighted in the depicted structure above by an asterisk.

Compounds of formula IA can occur as enantiomeric pure (+)-enantiomers (enantiomeric excess (ee)>99%) or as mixtures of the (+)- and (−)-enantiomers having an enantiomeric excess of the (+)-enantiomer.

Both enantiomers can be clearly distinguished by their optical activity [α]_{D} when dissolved in an achiral solvent: one has an optical activity [α]_{D} greater than 0° (the (+)-compound according to the invention) and one has an optical activity [α]_{D} lower than 0° (the (−)-compound according to the invention).

It has been found out that the (+)-compounds have higher microbiocidal activity as the (−)-compounds or as the racemic mixtures of both compounds.

The invention preferably provides compounds of the formula IA, wherein X₁ is hydrogen.

The invention preferably provides compounds of the formula IA, wherein at least one substituent X₂ is located in the ortho-position at the phenyl ring.

The invention preferably provides compounds of the formula IA, wherein each X₂ is chloro. In one embodiment of the invention n is 2. In another embodiment of the invention n is 3.

In one embodiment of the invention R₅₁ is methyl. In another embodiment of the invention R₅₁ is ethyl. In another embodiment of the invention R₅₁ is CF₃.

The invention preferably provides compounds of the formula IA with an enantiomeric excess of the (+)-enantiomer of at least 50%.

The invention preferably provides compound of the formula IA with an enantiomeric excess of the (+)-enantiomer of at least 75%.

In one embodiment of the invention, the compound of the formula IA is an enantiomeric pure (+)-enantiomer.

The compounds of formula IA may be prepared by reacting a racemic compound of formula IIk (which belongs to the group of compounds of formula II above)

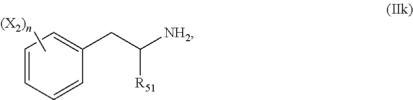

in which R₅₁, X₂ and n are as defined under formula IA and wherein said compound of formula IIk has an optical activity [α]_{D} of 0° when dissolved in an achiral solvent, with a compound of formula IIIk (which belongs to the group of compounds of formula IIIA above)

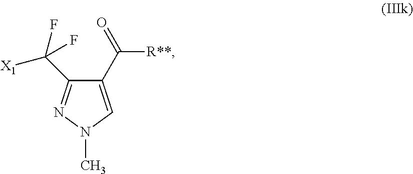

in which X₁ is as defined under formula IA, and R** is halogen, hydroxy or C₁₋₆ alkoxy, preferably chloro, in order to form a racemic compound of formula IA, followed by resolution of this racemic compound of formula IA by chromatography using a suited chiral stationary phase. An example of a suited chiral stationary phase is given in Example P15b).

The reaction between the compounds of formulae IIk and IIIk can be carried out as described for the reaction of compounds II and IIIA above.

Racemic intermediates of the formula IIk may be prepared according to reaction scheme 1 above or in analogy to this reaction scheme. In addition, intermediates of the formula III may also be prepared according to the following reaction scheme 12.

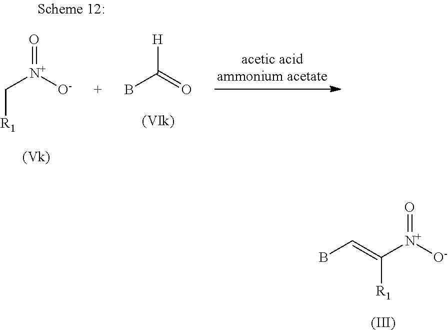

Nitroalkenes of formula III, in which B and R₁ are as defined under formula I, can be prepared by the reaction of a nitroalkane of formula Vk, in which R₁ is as defined under formula I, with a carbonyl compound of formula (VIk), in which B is as defined under formula I, in the presence of acetic acid and ammonium acetate at temperatures between ambient temperature and reflux temperature.

The compounds of formula IA may be prepared alternatively by reacting a compound of formula IIm

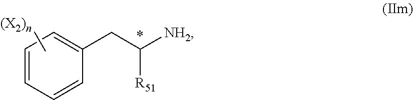

in which R₅₁, X₂ and n are as defined under formula IA and wherein said compound of formula IIm has an optical activity [α]_{D} of greater than 0° when dissolved in an achiral solvent; with a compound of formula IIIk as described above. Said intermediates of the formula IIm may be prepared by the resolution of the according racemic intermediates of formula IIk by chromatography using a suited chiral stationary phase. Intermediates of the formula IIm
are novel and were developed specifically for the preparation of compounds of formula IA. Accordingly, these intermediates also form part of the subject-matter of the invention.

As compounds of the formula IIIk belong to the group of compounds of formula IIIA, they are also known and some of them are commercially available. They can be prepared analogously as described, for example, in WO 93/11117. The compounds of formula Vk and VIk are known and are commercially available or can be prepared according to methods known in the art.

It has now been found that the compounds of formula IA have, for practical purposes, a very advantageous spectrum of activities for protecting useful plants against diseases that are caused by phytopathogenic microorganisams, such as fungi, bacteria or viruses.

The invention relates to a method of controlling or preventing infestation of useful plants by phytopathogenic microorganisms, wherein a compound of formula IA is applied as active ingredient to the plants, to parts thereof or the locus thereof. The compounds of formula IA according to the invention are distinguished by excellent activity at low rates of application, by being well tolerated by plants and by being environmentally safe. They have very useful curative, preventive and systemic properties and are used for protecting numerous useful plants. The compounds of formula IA can be used to inhibit or destroy the diseases that occur on plants or parts of plants (fruit, blossoms, leaves, stems, tubers, roots) of different crops of useful plants, while at the same time protecting also those parts of the plants that grow later e.g. from phytopathogenic microorganisms.

It is also possible to use compounds of formula IA as dressing agents for the treatment of plant propagation material, in particular of seeds (fruit, tubers, grains) and plant cuttings (e.g. rice), for the protection against fungal infections as well as against phytopathogenic fungi occurring in the soil.

Furthermore the compounds of formula IA according to the invention may be used for controlling fungi in related areas, for example in the protection of technical materials, including wood and wood related technical products, in food storage or in hygiene management.

The compounds of formula IA are, for example, effective against the phytopathogenic fungi of the following classes: Fungi imperfecti (e.g. Botrytis, Pyricularia, Helminthosporium, Fusarium, Septoria, Cercospora and Alternaria) and Basidiomycetes (e.g. Rhizoctonia, Hemileia, Puccinia). Additionally, they are also effective against the Ascomycetes classes (e.g. Venturia and Erysiphe, Podosphaera, Monilinia, Uncinula) and of the Oomycetes classes (e.g. Phytophthora, Pythium, Plasmopara). Outstanding activity has been observed against powdery mildew diseases (Uncinula necator). Furthermore, the novel compounds of formula IA are effective against phytopathogenic bacteria and viruses (e.g. against Xanthomonas spp, Pseudomonas spp, Erwinia amylovora as well as against the tobacco mosaic virus). Good activity has been observed against Asian soybean rust (Phakopsora pachyrhizi).

Within the scope of the invention, useful plants to be protected typically comprise the following species of plants: cereal (wheat, barley, rye, oat, rice, maize, sorghum and related species); beet (sugar beet and fodder beet); pomes, drupes and soft fruit (apples, pears, plums, peaches, almonds, cherries, strawberries, raspberries and blackberries); leguminous plants (beans, lentils, peas, soybeans); oil plants (rape, mustard, poppy, olives, sunflowers, coconut, castor oil plants, cocoa beans, groundnuts); cucumber plants (pumpkins, cucum-bers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocado, cinnamomum, camphor) or plants such as tobacco, nuts, coffee, eggplants, sugar cane, tea, pepper, vines, hops, bananas and natural rubber plants, as well as ornamentals.

The compounds of formula IA can be used in unmodified form or, preferably, together with carriers and adjuvants conventionally employed in the art of formulation. Therefore the invention also relates to compositions for controlling and protecting against phytopathogenic microorganisms, comprising a compound of formula IA and an inert carrier, and to a method of controlling or preventing infestation of useful plants by phytopathogenic microorganisms, wherein a composition, comprising a compound of formula IA as active ingredient and an inert carrier, is applied to the plants, to parts thereof or the locus thereof.

Further characteristics of compositions comprising compounds of formula IA, their application methods and their use rates are as described for compositions comprising compounds of formula I above. For use in the method according to the invention, the compounds of formula IA can be converted into the customary formulations described above, e.g. solutions, emulsions, suspensions, dusts, powders, pastes and granules. The use form will depend on the particular intended purpose; in each case, it should ensure a fine and even distribution of the compound of formula IA.

Compounds of formula IA can also be used in combination with glyphosate as described for compounds of formula I above. Said methods of using compounds of formula IA in combination with glyphosate are particularly effective against the phytopathogenic organisms of the kingdom Fungi, phylum Basidiomycot, class Uredinomycetes, subclass Urediniomycetidae and the order Uredinales (commonly referred to as rusts). Species of rusts having a particularly large impact on agriculture include those of the family Phakopsoraceae, particularly those of the genus Phakopsora, for example Phakopsora pachyrhizi, which is also referred to as Asian soybean rust, and those of the family Pucciniaceae, particularly those of the genus Puccinia such as Puccinia graminis, also known as stem rust or black rust, which is a problem disease in cereal crops and Puccinia recondita, also known as brown rust.

An embodiment of said method is a method of protecting crops of useful plants against attack by a phytopathogenic organism and/or the treatment of crops of useful plants infested by a phytopathogenic organism, said method comprising simultaneously applying glyphosate, including salts or esters thereof, and at least one compound of formula IA, which has activity against the phytopathogenic organism to at least one member selected from the group consisting of the plant, a part of the plant and the locus of the plant.

Surprisingly, it has now been found that the compounds of formula IA, or a pharmaceutical salt thereof, described above have also an advantageous spectrum of activity for the treatment and/or prevention of microbial infection in an animal.

According to the present invention there is provided the use of a compound of formula IA in the manufacture of a medicament for use in the treatment and/or prevention of microbial infection in an animal. There is also provided the use of a compound of formula IA as a pharmaceutical agent. There is also provided the use of a compound of formula IA as an antimicrobial agent in the treatment of an animal. According to the present invention there is also provided a pharmaceutical composition comprising as an active ingredient a compound of formula IA, or a pharmaceutically acceptable salt thereof, and a pharmaceutically acceptable diluent or carrier. This composition can be used for the treatment and/or prevention of antimicrobial infection in an animal. This pharmaceutical composition can be in a form suitable for oral administration, such as tablet, lozenges, hard capsules, aqueous suspensions, oily suspensions, emulsions dispersible powders, dispersible granules, syrups and elixirs. Alternatively this pharmaceutical composition can be in a form suitable for topical application, such as a spray, a cream or lotion. Alternatively this pharmaceutical composition can be in a form suitable for parenteral administration, for example injection. Alternatively this pharmaceutical composition can be in inhalable form, such as an aerosol spray.

The compounds of formula IA are effective against various microbial species able to cause a microbial infection in an animal. Examples of such microbial species are those causing Aspergillosis such as Aspergillus fumigatus, A. flavus, A. terrus, A. nidulans and A. niger, those causing Blastomycosis such as Blastomyces dermatitidis; those causing Candidiasis such as Candida albicans, C. glabrata, C. tropicalis, C. parapsilosis, C. krusei and C. lusitaniae; those causing Coccidioidomycosis such as Coccidioides immitis; those causing Cryptococcosis such as Cryptococcus neoformans; those causing Histoplasmosis such as Histoplasma capsulatum and those causing Zygomycosis such as Absidia corymbifera, Rhizomucor pusillus and Rhizopus arrhizus. Further examples are Fusarium Spp such as Fusarium oxysporum and Fusarium solani and Scedosporium Spp such as Scedosporium apiospermum and Scedosporium prolificans. Still further examples are Microsporum Spp, Trichophyton Spp, Epidermophyton Spp, Mucor Spp, Sporothorix Spp, Phialophora Spp, Cladosporium Spp, Petriellidium spp, Paracoccidioides Spp and Histoplasma Spp.

The following non-limiting Examples illustrate this aspect of the invention in greater detail without limiting it.

### PREPARATION EXAMPLES

### Example P15

### Preparation of (+)-3-difluoromethyl-1-methyl-1H-pyrazole-4-carboxylic acid [2-(2,4-dichlorophenyl)-1-methyl-ethyl]-amide (compound no. A1.01)

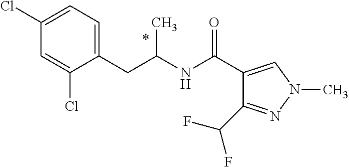

### a) Preparation of (+)-3-difluoromethyl-1-methyl-1H-pyrazole-4-carboxylic acid [2-(2,4-dichlorophenyl)-1-methyl-ethyl]-amide (compound A1.01)

Racemic 3-difluoromethyl-1-methyl-1H-pyrazole-4-carboxylic acid [2-(2,4-dichlorophenyl)-1-methyl-ethyl]-amide (480 mg, prepared as described in Example P2) was dissolved in n-hexane/isopropanol 3:1 (v/v) 72 ml. The solution was purified on Chiralpak AD® (Lot No. AD00CM-BF001 Daicel Japan, dimension: 500 mm×50 mm, particle size: 20 μm, flow rate: 30 ml/min) using n-hexane/isopropanol 7:3 (v/v) as eluant on high performance liquid chromatography (HPLC). For the separation of the whole material runs of 8 ml each (53 mg of the racemate) were separated on the column. The detection of the compounds was performed with UV detector at 210 nm. Pure enantiomeric samples (ee>99%) checked by analytical HPLC (Chiralpak AD00CE-CH017, Daicel) were combined and the solvent was evaporated.

Optical rotation data has been collected on a Perkin Elmer 241 Polarimeter (compounds were dissolved in CHCl₃, temperature is given in degrees Celsius; “c” stands for concentration in g/ml, the optical path length was 10 cm).

### Compound A1.01, (+)-Compound:

116 mg of (+)-3-difluoromethyl-1-methyl-1H-pyrazole-4-carboxylic acid [2-(2,4-dichlorophenyl)-1-methyl-ethyl]-amide was obtained in the form of a solid (Chiralpak AD00CE-CH017, Daicel, n-hexane/isopropanol 85:15; Retention time: 10.34 min); [α]²³_{D}=+50 (c 4.9, CHCl₃).

### Comparative Example: (−)-Analogue of Compound A1.01

174 mg of (+3-difluoromethyl-1-methyl-1H-pyrazole-4-carboxylic acid [2-(2,4-dichlorophenyl)-1-methyl-ethyl]-amide was obtained in the form of a solid (Chiralpak AD00CE-CH017, Daicel, n-hexane/isopropanol 85:15; Retention time: 7.80 min); [α]²³_{D}=−52 (c 4.4, CHCl₃).

### Table 15: Compounds of Formula IA

The invention is further illustrated by the preferred individual compounds of formula (IA) listed below in Table 15.

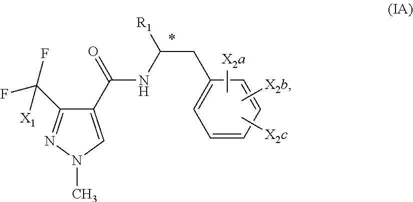

The [α]²³_{D} value is measured by dissolving the compound of formula IA in chloroform and measuring its optical activity at A=589 nm and 23° C. with an optical path length of 10 cm. “c” stands for concentration and is measured in g/ml.

TABLE 15

| Comp. No. | X₁ | R₁ | X₂ₐ | X_{2b} | X_{2c} | [α]²³_{D} |
|---|---|---|---|---|---|---|
| A1.01 | H | CH₃ | 2-Cl | 4-Cl | H | +50 |
| | | | | | | c = 4.9 |
| A1.02 | H | CH₃ | 2-Cl | 6-Cl | 4-Cl | — |
| A1.03 | H | CH₃ | 2-Cl | 6-Cl | H | — |
| A1.04 | H | CH₃ | 2-Cl | 6-CH₃ | H | — |
| A1.05 | H | CH₃ | 2-Cl | 6-CF₃ | H | — |
| A1.06 | H | CH₃ | 2-Cl | 6-Cl | 4-CF₃ | — |
| A1.07 | H | CH₃ | 2-Cl | 6-Cl | 4-CH₃ | — |
| A1.08 | H | CH₃ | 2-Cl | 6-Cl | 4-Br | — |
| A1.09 | F | CH₃ | 2-Cl | 4-Cl | H | — |
| A1.10 | F | CH₃ | 2-Cl | 6-Cl | 4-Cl | — |
| A1.11 | F | CH₃ | 2-Cl | 6-Cl | H | — |
| A1.12 | F | CH₃ | 2-Cl | 6-CH₃ | H | — |
| A1.13 | F | CH₃ | 2-Cl | 6-CF₃ | H | — |
| A1.14 | F | CH₃ | 2-Cl | 6-Cl | 4-CF₃ | — |
| A1.15 | F | CH₃ | 2-Cl | 6-Cl | 4-CH₃ | — |
| A1.16 | F | CH₃ | 2-Cl | 6-Cl | 4-Br | — |
| A1.17 | H | C₂H₅ | 2-Cl | 4-Cl | H | — |
| A1.18 | H | C₂H₅ | 2-Cl | 6-Cl | 4-Cl | — |
| A1.19 | H | C₂H₅ | 2-Cl | 6-Cl | H | — |
| A1.20 | H | C₂H₅ | 2-Cl | 6-CH₃ | H | — |
| A1.21 | H | C₂H₅ | 2-Cl | 6-CF₃ | H | — |
| A1.22 | H | C₂H₅ | 2-Cl | 6-Cl | 4-CF₃ | — |
| A1.23 | H | C₂H₅ | 2-Cl | 6-Cl | 4-CH₃ | — |
| A1.24 | H | C₂H₅ | 2-Cl | 6-Cl | 4-Br | — |

### Table 16: Compounds of Formula IIn

The invention is further illustrated by the preferred individual compounds of formula (IIn) listed below in Table 16.

TABLE 16
(IIn)

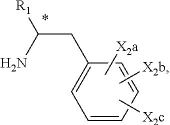

| Comp. | | | | | |
|---|---|---|---|---|---|
| No. | R₁ | X₂ₐ | X_{2b} | X_{2c} | [α]²³_{D} |
| Z2.01 | CH₃ | 2-Cl | 4-Cl | H | — |
| Z2.02 | CH₃ | 2-Cl | 6-Cl | 4-Cl | — |
| Z2.03 | CH₃ | 2-Cl | 6-Cl | H | — |
| Z2.04 | CH₃ | 2-Cl | 6-CH₃ | H | — |
| Z2.05 | CH₃ | 2-Cl | 6-CF₃ | H | — |
| Z2.06 | CH₃ | 2-Cl | 6-Cl | 4-CF₃ | — |
| Z2.07 | CH₃ | 2-Cl | 6-Cl | 4-CH₃ | — |
| Z2.08 | CH₃ | 2-Cl | 6-Cl | 4-Br | — |
| Z2.09 | CH₃ | 2-Cl | 4-Cl | H | — |
| 22.10 | CH₃ | 2-Cl | 6-Cl | 4-Cl | — |
| Z2.11 | CH₃ | 2-Cl | 6-Cl | H | — |
| Z2.12 | CH₃ | 2-Cl | 6-CH₃ | H | — |
| Z2.13 | CH₃ | 2-Cl | 6-CF₃ | H | — |
| Z2.14 | CH₃ | 2-Cl | 6-Cl | 4-CF₃ | — |
| Z2.15 | CH₃ | 2-Cl | 6-Cl | 4-CH₃ | — |
| Z2.16 | CH₃ | 2-Cl | 6-Cl | 4-Br | — |
| Z2.17 | C₂H₅ | 2-Cl | 4-Cl | H | — |
| Z2.18 | C₂H₅ | 2-Cl | 6-Cl | 4-Cl | — |
| Z2.19 | C₂H₅ | 2-Cl | 6-Cl | H | — |
| Z2.20 | C₂H₅ | 2-Cl | 6-CH₃ | H | — |
| Z2.21 | C₂H₅ | 2-Cl | 6-CF₃ | H | — |
| Z2.22 | C₂H₅ | 2-Cl | 6-Cl | 4-CF₃ | — |
| Z2.23 | C₂H₅ | 2-Cl | 6-Cl | 4-CH₃ | — |
| Z2.24 | C₂H₅ | 2-Cl | 6-Cl | 4-Br | — |

### BIOLOGICAL EXAMPLES

### Fungicidal Actions

### Example B-11: Action Against Botrytis cinerea—Fungal Growth Assay

Conidia of the fungus from cryogenic storage were directly mixed into nutrient broth (PDB potato dextrose broth). After placing a (DMSO) solution of the test compounds into a microtiter plate (96-well format) the nutrient broth containing the fungal spores was added. The test plates were incubated at 24° C. and the inhibition of growth was determined photometrically after 48-72 hrs. The activity of a compound is expressed as fungal growth inhibition (0=no growth inhibition, ratings of 80% to 99% mean good to very good inhibition, 100%=complete inhibition).

| | 20 | 6 | 2 |
|---|---|---|---|
| Compound | ppm | ppm | ppm |
| (+)-3-difluoromethyl-1-methyl-1H-pyrazole-4- | 100 | 100 | 80 |
| carboxylic acid [2-(2,4-dichlorophenyl)-1-methyl- | | | |
| ethyl]-amide (compound according to the invention) | | | |
| (−)-3-difluoromethyl-1-methyl-1H-pyrazole-4- | 20 | 20 | 0 |
| carboxylic acid [2-(2,4-dichlorophenyl)-1-methyl- | | | |
| ethyl]-amide (comparative example) | | | |

### Example B-12: Action Against Mycosphaerella arachidis (Early Leaf Spot of Groundnut; Cercospora arachidicola (Anamorph))—Fungal Growth Assay

Conidia of the fungus from cryogenic storage were directly mixed into nutrient broth (PDB potato dextrose broth). After placing a (DMSO) solution of the test compounds into a microtiter plate (96-well format) the nutrient broth containing the fungal spores was added. The test plates were incubated at 24° C. and the inhibition of growth was measured photometrically after 6-7 days. The activity of a compound is expressed as fungal growth inhibition (0=no growth inhibition, ratings of 80% to 99% mean good to very good inhibition, 100%=complete inhibition).

| | 20 | 6 | 2 |
|---|---|---|---|
| Compound | ppm | ppm | ppm |
| (+)-3-difluoromethyl-1-methyl-1H-pyrazole-4- | 100 | 100 | 100 |
| carboxylic acid [2-(2,4-dichlorophenyl)-1-methyl- | | | |
| ethyl]-amide (compound according to the invention) | | | |
| (−)-3-difluoromethyl-1-methyl-1H-pyrazole-4- | 80 | 50 | 0 |
| carboxylic acid [2-(2,4-dichlorophenyl)-1-methyl- | | | |
| ethyl]-amide (comparative example) | | | |

### Example B-13: Action Against Septoria tritici—Fungal Growth Assay

Conidia of the fungus from cryogenic storage were directly mixed into nutrient broth (PDB potato dextrose broth). After placing a (DMSO) solution of the test compounds into a microtiter plate (96-well format) the nutrient broth containing the fungal spores was added. The test plates were incubated at 24° C. and the inhibition of growth was determined photometrically after 72 hrs. The activity of a compound is expressed as fungal growth inhibition (0=no growth inhibition, ratings of 80% to 99% mean good to very good inhibition, 100%=complete inhibition).

| | 20 | 6 | 2 |
|---|---|---|---|
| Compound | ppm | ppm | ppm |
| (+)-3-difluoromethyl-1-methyl-1H-pyrazole-4- | 100 | 100 | 100 |
| carboxylic acid [2-(2,4-dichlorophenyl)-1-methyl- | | | |
| ethyl]-amide (compound according to the invention) | | | |
| (−)-3-difluoromethyl-1-methyl-1H-pyrazole-4- | 100 | 80 | 0 |
| carboxylic acid [2-(2,4-dichlorophenyl)-1-methyl- | | | |
| ethyl]-amide (comparative example) | | | |

### Example B-14: Action Against Monographella nivalis (Anamorph: Fusarium nivale, Microdochium nivale; Snow Mould)—Fungal Growth Assay

After placing a DMSO-solution (2% Dimethylsulfoxid, 0.025% Tween 20) of the test compounds into a microtiter plate (96-well format) the nutrient broth containing the fungal spores was added. 40.000 conidia/ml of the fungus from cryogenic storage were directly mixed into nutrient broth (PDB potato dextrose broth). The compounds were tested at a variety of application rates; these rates are shown in parts per million (ppm) in the table. The test plates were incubated at 24° C. and the inhibition of growth was measured photometrically after 72 hrs (0=no growth inhibition, ratings of 80% to 99% mean good to very good inhibition, 100%=complete inhibition).

| | 20 | 6 | 2 |
|---|---|---|---|
| Compound | ppm | ppm | ppm |
| (+)-3-difluoromethyl-1-methyl-1H-pyrazole-4- | 100 | 100 | 100 |
| carboxylic acid [2-(2,4-dichlorophenyl)-1-methyl- | | | |
| ethyl]-amide (compound according to the invention) | | | |
| (−)-3-difluoromethyl-1-methyl-1H-pyrazole-4- | 20 | 0 | 0 |
| carboxylic acid [2-(2,4-dichlorophenyl)-1-methyl- | | | |
| ethyl]-amide (comparative example) | | | |

### Example B-15: Action Against Pyrenophora teres (Net Blotch) on Barley

Barley leaf segments are placed on agar in multiwell plates (24-well format) and sprayed with test solutions (200 ppm, 60 pp, and 20 ppm of active ingredient). After drying, the leaf disks are inoculated with a spore suspension of the fungus. After appropriate incubation the activity of a compound is assessed 4 days after inoculation as preventive fungicidal activity (in %).

| | 200 | 60 | 20 |
|---|---|---|---|
| Compound | ppm | ppm | ppm |
| (+)-3-difluoromethyl-1-methyl-1H-pyrazole-4- | 100 | 100 | 100 |
| carboxylic acid [2-(2,4-dichlorophenyl)-1-methyl- | | | |
| ethyl]-amide (compound according to the invention) | | | |
| (−)-3-difluoromethyl-1-methyl-1H-pyrazole-4- | 100 | 100 | 80 |
| carboxylic acid [2-(2,4-dichlorophenyl)-1-methyl- | | | |
| ethyl]-amide (comparative example) | | | |

What is claimed is:

1. A compound of the formula I 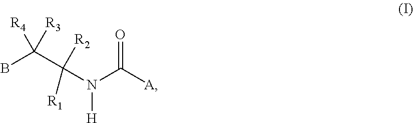
wherein
R₁, R₂, R₃ and R₄ independently of each other stand for hydrogen, halogen, nitro, C₁-C₆alkyl, which is unsubstituted or substituted by one or more substituents R₅, C₃-C₆cycloalkyl, which is unsubstituted or substituted by one or more substituents R₅, C₂-C₆alkenyl, which is unsubstituted or substituted by one or more substituents R₅ or C₂-C₆alkynyl, which is unsubstituted or substituted by one or more substituents R₅;
or R₁ and R₂ together are a C₂-C₅alkylene group, which is unsubstituted or substituted by one or more C₁-C₆alkyl groups;
or R₃ and R₄ together are a C₂-C₅alkylene group, which is unsubstituted or substituted by one or more C₁-C₆alkyl groups;
each R₅ independently of each other stands for halogen, nitro, C₁-C₆alkoxy, C₁-C₆halogenalkoxy, C₃-C₆cycloalkyl, C₁-C₆alkylthio, C₁-C₆halogenalkylthio or —C(R^{a})═N(OR^{b}));
R^{a} is hydrogen or C₁-C₆alkyl;
R^{b} is C₁-C₆alkyl;
A is A₁ 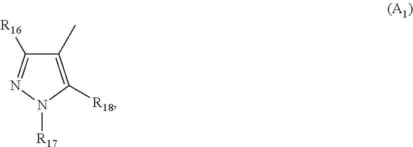
in which
R₁₆ is halogenmethyl;
R₁₇ is C₁-C₄alkyl, C₁-C₄halogenalkyl, C₁-C₄alkoxy-C₁-C₄alkyl or C₁-C₄halogenalkoxy-C₁-C₄alkyl; and
R₁₈ is hydrogen, halogen, cyano, nitro, C₁-C₄alkyl, C₁-C₄halogenalkyl, C₁-C₄halogenalkoxy, C₁-C₄alkoxy-C₁-C₄alkyl or C₁-C₄halogenalkoxy-C₁-C₄alkyl;
or A is A₂ 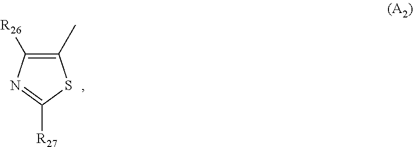
in which
R₂₆ is halogenmethyl; and
R₂₇ is C₁-C₄alkyl, C₁-C₄halogenalkyl, C₁-C₄alkoxy-C₁-C₄alkyl or C₁-C₄halogenalkoxy-C₁-C₄alkyl;
or A is A₃ 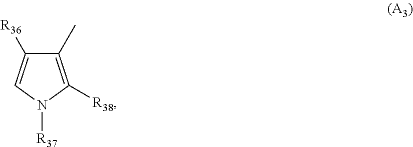
in which
R₃₆ is halogenmethyl;
R₃₇ is C₁-C₄alkyl, C₁-C₄halogenalkyl, C₁-C₄alkoxy-C₁-C₄alkyl or C₁-C₄halogenalkoxy-C₁-C₄alkyl; and
R₃₈ is hydrogen, halogen, cyano, nitro, C₁-C₄alkyl, C₁-C₄halogenalkyl, C₁-C₄halogenalkoxy, C₁-C₄alkoxy-C₁-C₄alkyl or C₁-C₄halogenalkoxy-C₁-C₄alkyl;
or A is A₄ 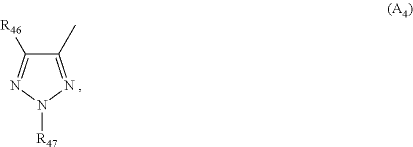
in which
R₄₈ is halogenmethyl; and
R₄₇ is C₁-C₄alkyl, C₁-C₄halogenalkyl, C₁-C₄alkoxy-C₁-C₄alkyl or C₁-C₄halogenalkoxy-C₁-C₄alkyl;
B is a phenyl, naphthyl or quinolinyl group, which is substituted by one or more substituents R₈;
each substituent R₈ independently of each other stands for halogen, C₁-C₆haloalkoxy, C₁-C₆haloalkylthio, cyano, nitro, —C(R^{c})═N(OR^{d}), C₁-C₆alkyl, which is unsubstituted or substituted by one or more substituents R₉, C₃-C₆cycloalkyl, which is unsubstituted or substituted by one or more substituents R₉, C₆-C₁₄ bicycloalkyl, which is unsubstituted or substituted by one or more substituents R₉, C₂-C₆alkenyl, which is unsubstituted or substituted by one or more substituents R₉, C₂-C₆alkynyl, which is unsubstituted or substituted by one or more substituents R₉, phenyl, which is unsubstituted or substituted by one or more substituents R₉ or phenoxy, which is unsubstituted or substituted by one or more substituents R₉, pyridinyloxy, which is unsubstituted or substituted by one or more substituents R₉;
each R^{c} is independently of each other hydrogen or C₁-C₆alkyl;
each R^{d} is independently of each other C₁-C₆alkyl;
each R₉ is independently of each other halogen, nitro, C₁-C₆alkoxy, C₁-C₆halogenalkoxy, C₁-C₆alkylthio, C₁-C₆halogenalkylthio, C₃-C₆alkenyloxy, C₃-C₆alkynyloxy or —C(R^{e})═N(OR^{f});
each R^{e} is independently of each other hydrogen or C₁-C₆alkyl;
each R^{f} is independently of each other C₁-C₆alkyl;
and tautomers/isomers/enantiomers of these compounds.

2. A compound of formula I according to claim 1, wherein
R₁, R₂, R₃ and R₄ independently of each other stand for hydrogen, halogen, nitro, C₁-C₆alkyl, which is unsubstituted or substituted by one or more substituents R₅, C₃-C₆cycloalkyl, which is unsubstituted or substituted by one or more substituents R₅, C₂-C₆alkenyl, which is unsubstituted or substituted by one or more substituents R₅ or C₂-C₆alkynyl, which is unsubstituted or substituted by one or more substituents R₅;
or R₁ and R₂ together are a C₂-C₅alkylene group, which is unsubstituted or substituted by one or more C₁-C₆alkyl groups;
or R₃ and R₄ together are a C₂-C₅alkylene group, which is unsubstituted or substituted by one or more C₁-C₆alkyl groups;
each R₅ independently of each other stands for halogen, nitro, C₁-C₆alkoxy, C₁-C₆halogenalkoxy, C₃-C₆cycloalkyl, C₁-C₆alkylthio, C₁-C₆halogenalkylthio or —C(R^{a})═N(OR^{b});
R^{a} is hydrogen or C₁-C₆alkyl;
R^{b} is C₁-C₆alkyl;
B is a phenyl, naphthyl or quinolinyl group, which is substituted by one or more substituents R₈;
each substituent R₈ independently of each other stands for halogen, C₁-C₆haloalkoxy, C₁-C₆haloalkylthio, cyano, nitro, —C(R^{c})═N(OR^{d}), C₁-C₆alkyl, which is unsubstituted or substituted by one or more substituents R₉, C₃-C₆cycloalkyl, which is unsubstituted or substituted by one or more substituents R₉, C₆-C₁₄bicycloalkyl, which is unsubstituted or substituted by one or more substituents R₉, C₂-C₆alkenyl, which is unsubstituted or substituted by one or more substituents R₉, C₂-C₆alkynyl, which is unsubstituted or substituted by one or more substituents R₉, phenyl, which is unsubstituted or substituted by one or more substituents R₉;
each R^{c} is independently of each other hydrogen or C₁-C₆alkyl;
each R^{d} is independently of each other C₁-C₆alkyl;
each R₉ is independently of each other halogen, nitro, C₁-C₆alkoxy, C₁-C₆halogenalkoxy, C₁-C₆alkylthio, C₁-C₆halogenalkylthio, C₃-C₆alkenyloxy, C₃-C₆alkynyloxy or —C(R^{e})═N(OR^{f});
each R^{e} is independently of each other hydrogen or C₁-C₆alkyl; and
each R^{f} is independently of each other C₁-C₆alkyl.

3. A compound of formula I according to claim 2, wherein A is Al.

4. A compound of formula I according to claim 2, wherein B is a phenyl group, which is substituted by one or more substituents R₈.

5. A compound of formula I according to claim 4, wherein each substituent R₈ independently of each other stands for halogen, C₁-C₆haloalkoxy, C₁-C₆haloalkylthio, nitro, —C(R^{c})═N(OR^{d}), C₁-C₆alkyl, which is unsubstituted or substituted by one or more substituents R₉, C₂-C₆alkenyl, which is unsubstituted or substituted by one or more substituents R₉ or C₂-C₆alkynyl, which is unsubstituted or substituted by one or more substituents R₉.

6. A compound according to claim 2, wherein A is Al and B is phenyl group, which is substituted by one or more substituents R₈, wherein each substituent R₈ independently of each other stands for halogen, C₁-C₆haloalkoxy, C₁-C₆haloalkylthio, nitro, —C(R^{c})═N(OR^{d}), C₁-C₆alkyl, which is unsubstituted or substituted by one or more substituents R₉, C₂-C₆alkenyl, which is unsubstituted or substituted by one or more substituents R₉ or C₂-C₆alkynyl, which is unsubstituted or substituted by one or more substituents R₉.

7. A compound of formula I according to claim 4, wherein B is B₁ 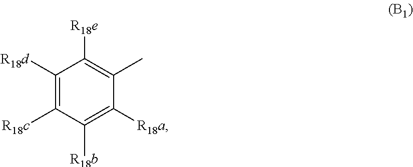
in which R₁₈ₐ is hydrogen, halogen, cyano, C₁-C₆alkyl, C₂-C₆alkynyl, C₁-C₆alkoxy, C₁-C₆halogenalkyl, C₁-C₆halogenalkoxy or phenyl, which is unsubstituted or substituted by one or more halogens; R_{18b} is hydrogen, halogen, cyano, C₁-C₆alkyl, C₂-C₆alkynyl, C₁-C₆alkoxy, C₁-C₆halogenalkyl, C₁-C₆halogenalkoxy or phenyl, which is unsubstituted or substituted by one or more halogens; R_{18c} is hydrogen, halogen, cyano, C₁-C₆alkyl, C₂-C₆alkynyl, C₁-C₆alkoxy, C₁-C₆halogenalkyl, C₁-C₆halogenalkoxy or phenyl, which is unsubstituted or substituted by one or more halogens; R_{18d} is hydrogen, halogen, cyano, C₁-C₆alkyl, C₂-C₆alkynyl, C₁-C₆alkoxy, C₁-C₆halogenalkyl, C₁-C₆halogenalkoxy or phenyl, which is unsubstituted or substituted by one or more halogens; R₁₈ₑ is hydrogen, halogen, cyano, C₁-C₆alkyl, C₂-C₆alkynyl, C₁-C₆alkoxy, C₁-C₆halogenalkyl, C₁-C₆halogenalkoxy or phenyl, which is unsubstituted or substituted by one or more halogens; provided that at least one of R₁₈ₐ, R_{18b}, R_{18c}, R_{18d} and R₁₈ₐ is not hydrogen.

8. A compound of formula I according to claim 7, wherein R_{18b} and R_{18d} is hydrogen; and R₁₈ₐ, R_{18c} and R₁₈ₑ independently of one another are selected from hydrogen, halogen, C₂-C₆alkynyl or C₁-C₆halogenalkyl; provided that at least one of R₁₈ₐ, R_{18c} and R₁₈ₑ is not hydrogen.

9. A compound of formula I according to claim 2, wherein R₁, R₂, R₃ and R₄ independently of each other stands for hydrogen, halogen, nitro, C₁-C₆alkyl, which is unsubstituted or substituted by one or more substituents R₅, C₃-C₆cycloalkyl, which is unsubstituted or substituted by one or more substituents R₅, C₂-C₆alkenyl, which is unsubstituted or substituted by one or more substituents R₅ or C₂-C₆alkynyl, which is unsubstituted or substituted by one or more substituents R₅.

10. A compound of formula I according to claim 2, wherein R₁, R₂, R₃ and R₄ independently of each other stands for hydrogen, halogen or C₁-C₆alkyl, which is unsubstituted or substituted by one or more substituents selected from halogen, C₁-C₆alkoxy and C₁-C₆halogenalkoxy.

11. A compound of formula I according to claim 2, wherein R₁, R₂, R₃ and R₄ independently of each other stands for hydrogen, halogen or C₁-C₆alkyl, which is unsubstituted or substituted by one or more substituents selected from halogen and C₁-C₆alkoxy.

12. A compound of formula I according to claim 2, wherein R₁, R₂, R₃ and R₄ independently of each other stands for hydrogen, halogen, or C₁-C₆alkyl.

13. A compound of formula I according to claim 2, wherein R₁ is C₁-C₆alkyl or C₁-C₆haloalkyl.

14. A compound of formula I according to claim 2, wherein R₁ is halogen. or C₁-C₆alkyl.

15. A compound of formula I according to claim 2, wherein R₁ is C₁-C₆alkyl.

16. A compound of formula I according to claim 2, wherein R₃ is halogen.

17. A compound of formula I according to claim 2, wherein R₁ is CF₃, CF₂H or CFH₂.

18. A compound of formula I according to claim 2, wherein R₁ is CF₃.

19. A compound of formula I according to claim 2, wherein R₁ is CF₂H.

20. A compound of formula I according to claim 2, wherein R₁ is CFH₂.

21. A method of controlling or preventing infestation of useful plants by phytopathogenic microorganisms, wherein a compound of formula I according to claim 1 or a composition, comprising this compound as active ingredient, is applied to the plants, to parts thereof or the locus thereof.

22. A composition for controlling and protecting against phytopathogenic microorganisms, comprising a compound of formula I according to claim 1 and an inert carrier.

23. A compound of the formula IA 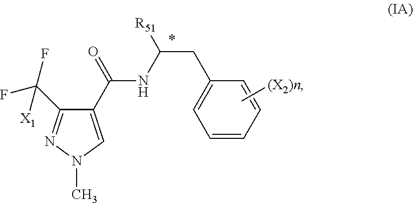
wherein R₅₁ is C₁-C₃alkyl, CF₃ or CF₂H; X₁ is hydrogen or fluoro; n is 2 or 3; each X₂ independently of each other stands for chloro, bromo, fluoro, CH₃ or CF₃;
having an optical activity [α]_{D} of greater than 0° when dissolved in an achiral solvent.

24. A compound of formula IA according to claim 23, wherein X₁ is hydrogen.

25. A compound of formula IA according to claim 23, wherein R₅₁ is methyl.

26. A compound of formula IA according to claim 23, wherein X₂ is chloro.

27. A compound of formula IA according to claim 23, wherein n is 2.

28. A compound of formula IA according to claim 23, wherein n is 3.

29. A method of controlling or preventing infestation of useful plants by phytopathogenic microorganisms, wherein a compound of formula IA according to claim 23 or a composition, comprising this compound as active ingredient, is applied to the plants, to parts thereof or the locus thereof.

30. A composition for controlling and protecting against phytopathogenic microorganisms, comprising a compound of formula IA according to claim 23 and an inert carrier.
